(19) United States
(12) Patent Application Publication
(10) Pub. No.: US 2015/0037375 A1
(43) Pub. Date: Feb. 5, 2015

(54) DOPING AGENTS AND POLYMERIC COMPOSITIONS THEREOF FOR CONTROLLED DRUG DELIVERY

(71) Applicants: Mark W. Grinstaff, Brookline, MA (US); Jesse Wolinsky, Brookline, MA (US); Stefan Yohe, Cambridge, MA (US); Jonah Andrew Kaplan, Newton, MA (US); Eric John Falde, Maplewood, MN (US); Joseph Steven Hersey, Boston, MA (US)

(72) Inventors: Mark W. Grinstaff, Brookline, MA (US); Jesse Wolinsky, Brookline, MA (US); Stefan Yohe, Cambridge, MA (US); Jonah Andrew Kaplan, Newton, MA (US); Eric John Falde, Maplewood, MN (US); Joseph Steven Hersey, Boston, MA (US)

(73) Assignee: TRUSTEES OF BOSTON UNIVERSITY ET AL, Boston, MA (US)

(21) Appl. No.: 14/233,570

(22) PCT Filed: Jul. 19, 2012
(86) PCT No.: PCT/US2012/047398

§ 371 Date: Sep. 22, 2014

Related U.S. Application Data
(60) Provisional application No. 61/582,937, filed on Jan. 4, 2012.
(60) Provisional application No. 61/509,281, filed on Jul. 19, 2011.

(51) Int. Cl. A61L 31/14; A61L 31/16; A61L 31/06

(52) U.S. Cl. CPC A61L 31/148; A61L 31/06; A61L 31/16; A61L 31/146; A61L 2300/216; A61L 2300/416
USPC 424/400; 514/283

(57) ABSTRACT

Provided herein are 3-dimensional drug-eluting materials comprising biodegradable polymer(s), one or more bioactive agents and entrapped air. Various embodiments of the methods and compositions described herein are based, in part, on the discovery of hydrophobic doping agents that can be used in the manufacture of polymeric drug delivery compositions that permit the encapsulation of air, thereby permitting tunable drug release via controlled air removal. Such compositions are particularly useful for delivering therapeutically effective doses of one or more bioactive agents to a subject over an extended period of time (e.g., days, weeks, or months).

### FIELD OF THE INVENTION

The field of the invention relates generally to drug delivery compositions and methods for their use.

### BACKGROUND

Cancer is responsible for over 1.5 million deaths per year in the US, or roughly one quarter of all reported deaths. Major sources of treatment failure include difficulties achieving therapeutic concentrations of chemotherapy at the site of disease, and high rates of relapse or recurrence. Polymeric delivery systems have been widely investigated as a means of delivering high concentrations of chemotherapy directly to the tumor site in cancer patients. These technologies aim to improve overall survival and quality of life by increasing the bioavailability of drug to the disease site while limiting systemic exposure and thus minimizing the severe systemic side effects associated with intravenous chemotherapy.

Over half of all new cancer diagnoses are discovered at an early stage, with no evidence of metastatic spreading. The standard of care for these patients is surgical resection with curative intent. Unfortunately, local recurrence remains a main source of treatment failure for most types of malignancies due to undetected remaining residual cancer cells following surgery. This is a particular problem for lung cancer patients, where the extent of surgical resection is limited by the need to conserve pulmonary function, leading to a dismal 40-60% 5-year survival for those patients diagnosed and surgically treated when the disease is localized and not clinically recognized as metastatic. Preventative chemotherapy and/or radiation have not become a standard of care treatment due to the associated substantial side effects to these therapies and difficulty predicting which patients would benefit.

A wide variety of polymeric compositions and drug delivery approaches have been developed in an attempt to address localized delivery to treat cancer patients. One strategy is to use nano-materials such as nanoparticles, liposomes, and dendrimers to localize to solid tumors via the Enhanced Permeability and Retention Effect by passive diffusion via leaky tumor vasculature. Other drug nano-carriers are conjugated with targeting moieties with an affinity for over-expressed tumor cell markers. A second strategy involves direct implantation of controlled release drug delivery depot systems. These technologies have been embodied in a variety of form-factors such as drug-eluting films, gels, wafers, rods, and particles and feature a range of predictable and prolonged drug release kinetics.

The pharmaceutical industry utilizes blended polymer coatings to achieve different types of release profiles such as linear, cyclic, or sigmoidal release from oral solid dosage delivery systems. Gastro-intestinal-tract (GIT) soluble and insoluble polymers have been mixed to afford great control of sustained drug delivery formulations over a 24 hour period (Siepmann, F., et al., J Control Release, 125: 1-15 (2008)). Increasing the mass ratio in favor of the more hydrophilic polymer of a blended composition is a common method of accelerating the release rate due to increased water uptake, swelling, and mechanical disruption of the polymer matrix. In one example, increasing the fraction of hydrophilic ammonium groups in poly(ethyl acrylate-co-methyl methacrylateco-trimethylammonioethyl methacrylate chloride) blends of varying ratios enhances polymer chain mobility, leading to nearly zero order release rates from <5% for low ammonium content blends to >80% cumulative release per hour when ammonium content is increased. Ethylcellulose is an impermeable GIT-insoluble polymer that is frequently blended with hydrophilic polymers to expedite release. Hydrophilic hydroxypropyl methylcellulose (HPMC) can be added to ethylcellulose as a leachable, leaving behind pores once exposed to aqueous media, and thus increasing water permeability into the remaining ethylcellulose (Frohoff-Hulsmann, M. A., et al., European Journal of Pharmaceutics and Biopharmaceutics, 48: 67-75 (1999)). Increasing the molecular weight of ethylcellulose acts as a mechanical stabilizer, protecting the matrix from the formation of cracks and fissures, and shifting the mechanism of release to diffusion controlled (Rowe, R. C. International Journal of Pharmaceutics, 29: 37-41, (1986)). The incorporation of hydrophilic PVA-PEG copolymer into ethylcellulose blends has a similar effect as HPMC, dramatically accelerating release rates with 5% incremental increases in PVA-PEG content. For example, <5% theophylline release is achieved after 7 hours with blends containing 5 wt % PVA-PEG, but is increased to >60% cumulative release when 15 wt % PVA-PEG is introduced to the blend (Siepmann, F., Journal of Controlled Release, 119: 182-189 (2007)).

Diffusion-mediated release from slow-degrading polymer is often logarithmic, with the release kinetic gradually decreasing over days to weeks. Conversely, hydrophobic drug release from rapid-degrading polymers like PLGA can either be too slow initially, or too quick as the onset of bulk degradation occurs. Many drugs are most effective at concentrations that fall within a therapeutic window—lower concentrations are not effective, and higher concentrations may elicit side effects/toxicities.

While oral solid dose delivery systems have their drug release kinetics tuned via polymer blending to deliver their payload over hours, the criteria for long-term controlled release from polymer implants is often different, relying more so on controlling the mechanism of degradation (diffusion vs. degradation mediated). Release kinetics from slow-degrading (>1 year) hydrophobic polymers such as poly(caprolactone) (PCL) and poly(trimethylene carbonate) (PTMC) are dominated by the rate-limiting diffusion of drug from the polymer matrix. Conversely, rapid degrading polymers (<6 month) such as poly(lactide) (PLA) and poly(lactide-co-glycolide) (PLGA) feature three step sigmoidal release kinetics beginning with water penetration into the matrix, followed by degradation-dependent “relaxation of the network” allowing for drug dissolution, and finally drug diffusion into the surrounding medium (Lao, L. L., et al. European Journal of Pharmaceutics and Biopharmaceutics, 70: 796-803 (2008)). These two classes of polymers have traditionally been blended in an effort to achieve intermediary release approximating zero-order release kinetics.

Long-term drug release kinetics from hydrophobic polymer blends is dependent on several factors including the hydrophobicity of the drug, individual polymer degradation rates, water permeability, and drug-polymer interactions. Several groups have developed predictive models to characterize how the independent properties of two systems would be affected via blending. One heuristic mathematical model was developed to predict release from polymer blends and compared to empirical release data of paclitaxel from blends of poly(caprolactone) and poly(lactide-co-glycolide). The model characterizes release as a 3-step process beginning with water penetration, degradation-dependent drug dissolution, and drug diffusion. The release kinetics from blends of these polymers ranging from 50 to 65% poly(lactide-co-glycolide) closely followed the predictive model. The first phase of release (˜5 days) was dominated by PCL component, since the barrier of water penetration is a rate-limiting step for glassy PLGA. The second phase of release (˜30 days) was long and gradual, dominated by the relaxation of the PLGA component of the network due to PLGA degradation. Finally, the third phase of release (˜10 days) occurs as bulk degradation of PLGA leaves PCL as the dominant remaining polymer, removing the barrier of diffusion for the remaining paclitaxel. This model breaks down once one of the two polymer components comprises ≦25% of the blend, whereas the dominant polymer tends to dictate the overall release kinetics. This phenomena is attributed to a loss of interconnectivity between the two polymers, resulting in micro-domains of the minority composition, and a lack of blended intermediary release kinetics (Lao, L. L., et al. European Journal of Pharmaceutics and Biopharmaceutics, 70: 796-803 (2008)). Thus, blending 25% of PCL with 75% PLGA results in release kinetics that were dominated by PLGA.

Agreeing with these predictive principles, poly(caprolactone) (PCL) blended with either poly(lactide) (PLA), or with poly(caprolactone-co-lactide) copolymer (R_{50:50} or R_{25:75}) demonstrates nearly two-fold slower release kinetics of northindrone with increasing lactide content (0-38 mol % lactide), as well as faster degradation rates due to the lactide component. (PCL/R_{50:50}) compared to the immiscible blends (PCL/PLA, PCL/R_{25:75}) (Shen, Y., et al. J Biomed Mater Res, 50: 528-535 (2000)). In a different type of example, lauryl ester-terminated PLGA oligomer and hydrophilic low molecular weight Pluronic F-127 were blended into PLGA to modulate degradation kinetics. Incorporation of Pluronic F-127 increased water uptake in blended polymer coatings (increase from ˜7% non-doped to 25%) while adding the hydrophobic oligomer decreased water content (reduction from ˜7 to 4%). Cumulative release of a lysozyme protein followed the 3-phase release typical from PLGA polymers. Onset of more rapid release resulting from bulk degradation (second phase of release) was accelerated when PLGA was doped with 6% Pluronic initiating at ˜7 days compared to 10 days. Doping PLGA with 30% hydrophobic PLGA oligomer delayed onset by about 5 days. For all three polymer groups, cumulative release terminated at about 30 days (Raiche, A. T. and Puleo, D. A. Int J Pharm, 311: 40-49 (2006)).

Two blending methods used to increase degradation rate, burst release, or shorten the total duration of release of hydrophobic drugs from hydrophobic aliphatic degradable polymers include incorporating hydrophilic polymers or doping with low molecular weight polymers. Both have the effect of leaving behind a porous network after diffusing rapidly away from the polymer blend depot. In one example, adipic anhydride (AA), with a rapid degradation rate over just several days, was blended into slow-degrading poly(trimethylene carbonate) (pTMC) to increase degradation and drug release rates. Blended disks tended to lose mass over the first 100 hours of degradation in direct proportion to the amount of AA incorporated into composite, with the exception of samples containing no more than 20 wt % of a low molecular weight pTMC component (17 kDa vs. 63.5 or 150 kDa), which disintegrated completely over the same time scale. Additionally, burst release of amitryptiline could be modulated between 20% cumulative release up to 100% over the first 100 hours by increasing the ratio of AA in the blend. Release rates were not significantly affected (Edlund, U. and Albertsson, A. C. Journal of Applied Polymer Science, 72: 227-239 (1999)). In another example, initial burst release of the bacteriocin plantaracin 423 decreased from approximately 70 to 55% of total drug loaded into poly(ethylene oxide)/poly(D,L-lactide) blend nanofiber meshes when the fraction of poly(ethylene oxide) was decreased from 90% to 50%. It was hypothesized that hydrophilic poly(ethylene glycol) swells the fibers, allowing for a larger burst. Release rates following the first 24 hours were not significantly affected by the blending ratio (Heunis, T., Int J Mol Sci, 12: 2158-2173).

Doping with low molecular weight PLGA (<10 kDa) into high molecular weight PLGA (˜100 kDa) microspheres has a significant effect on the diffusion-limiting stage of ganciclovir release. Before introducing low molecular weight PLGA, release is characterized by a rapid burst followed by prolonged slow release (<5% cumulative drug release over 60 days), and finally rapid release due to bulk degradation. Doping with as much as 75 wt % low molecular weight PLGA decreases the diffusion phase of release from 60 days to nearly 0 as the large component of low molecular weight polymer degrades and erodes much more rapidly (Duvvuri, S., Pharm Res, 23: 215-223 (2006)). In another instance of doping low molecular weight PLGA (8 kDa) into high molecular weight PLGA (28 kDa), the cumulative burst release of a peptide from microparticles increased from less than 10% to >50% over the first 24 hours (Ravivarapu, H. B., et al. Eur J Pharm Biopharm, 50: 263-270 (2000)).

Blending with hydrophilic polymers can have the opposite effect on protein release kinetics from hydrophobic polymer by increasing the partition coefficient of hydrophilic polypeptides to favor the blended polymer matrix. In one example, the large burst release of hydrophilic proteins from hydrophobic polylactide is moderated by blending the polymer with hydrophilic pluronics (poly(ethylene oxide-co-propylene oxide)). Cumulative release of bovine serum albumin over the first 48 hours was reduced from 95% to approximately 20% as the percentage of pluronic in the blend was increased from 0-30%. Degradation and long-term release rates were not dramatically affected. Conversely, the introduction of poly(propylene fumarate) increases the cumulative burst release of a model protein from PLGA microspheres from ˜15 to 65%, while decreasing the total degradation time of microparticle. Both trends were attributed to poly(propylene fumarate) being the more hydrophobic of the two polymers, thus presumably having a lower partition coefficient for the hydrophilic protein and also being less permeable to water penetration (Kempen, D. H et al., J Biomed Mater Res A, 70: 293-302 (2004)).

### SUMMARY

Provided herein are 3-dimensional drug delivery compositions with superior time-release properties that permit the release of one or more bioactive agents to a subject over extended time periods (e.g., 1 hour to 100 days). Also provided herein are methods for making and using such compositions.

One aspect provided herein relates to a 3 dimensional composition comprising: a) a biodegradable polymer; b) at least one bioactive agent; and c) a hydrophobic doping agent, wherein the surface hydrophobicity of the composition is substantially homogenous throughout the bulk of the composition, and wherein the composition comprises entrapped air.

In one embodiment of this aspect and all other aspects described herein, the presence of the hydrophobic doping agent increases the contact angle of said composition by at least 10 degrees, as compared with the same composition comprising the biodegradable polymer and the one or more bioactive agents but in the absence of the hydrophobic doping agent.

In another embodiment of this aspect and all other aspects described herein, the presence of the hydrophobic doping agent reduces the total amount of bioactive agent released from the composition by at least 20 percent over the first 24 hours, as compared with the same composition lacking the block co-polymer.

In another embodiment of this aspect and all other aspects described herein, the presence of the hydrophobic doping agent reduces the total amount of bioactive agent released from the composition by at least 20 percent over the first 10 days, as compared with the same composition lacking the hydrophobic doping agent.

In another embodiment of this aspect and all other aspects described herein, the composition comprises a nanofiber, a microfiber, and/or a bead structure.

In another embodiment of this aspect and all other aspects described herein, the composition exhibits tunable drug release via controlled air removal.

In another embodiment of this aspect and all other aspects described herein, the composition comprises between 0.01% and 50% hydrophobic doping agent by weight.

In another embodiment of this aspect and all other aspects described herein, the nanofiber or microfiber comprises a median diameter of between 10 nanometers and 100 micrometers.

In another embodiment of this aspect and all other aspects described herein, the at least one bioactive agent is selected from the group consisting of: an antibiotic, an antimitotic, an anti-inflammatory agent, a growth factor, a targeting compound, a cytokine, an immunotoxin, an anti-tumor antibody, an anti-angiogenic agent, an anti-edema agent, a radiosensitizer and a chemotherapeutic.

In another embodiment of this aspect and all other aspects described herein, the at least one bioactive agent is selected from the group consisting of a taxane, a camptothecin, and a platinum-containing molecule.

In another embodiment of this aspect and all other aspects described herein, the taxane comprises paclitaxel or docetaxol.

In another embodiment of this aspect and all other aspects described herein, the camptothecin comprises irinotecan, topotecan, or SN-38.

In another embodiment of this aspect and all other aspects described herein, the platinum-containing molecule comprises carboplatin or cisplatin.

In another embodiment of this aspect and all other aspects described herein, the composition releases the at least one bioactive agent over a period effective to inhibit or delay tumor growth or inhibit or delay tumor metastasis when administered to a subject.

In another embodiment of this aspect and all other aspects described herein, the composition is administered in close proximity to a tumor in the subject.

In another embodiment of this aspect and all other aspects described herein, the composition releases the at least one bioactive agent over a period effective to inhibit or delay tumor recurrence when administered to a subject.

In another embodiment of this aspect and all other aspects described herein, the composition is administered by affixing the composition to a tumor resection margin following surgery.

In another embodiment of this aspect and all other aspects described herein, wherein the composition releases the at least one bioactive agent continuously at a therapeutic dose for at least 7 days.

In another embodiment of this aspect and all other aspects described herein, the biodegradable polymer is selected from the group consisting of a polyester, a polycarbonate, a polyamide, a polyether, a polyanhydride, and a copolymer or blend thereof.

In another embodiment of this aspect and all other aspects described herein, the biodegradable polymer is selected from the group consisting of poly(caprolactone), poly(lactide-co-glycolide), poly(dioxanone), poly(trimethylene carbonate), poly(ethylene glycol), poly(glycerol monostearate-co-caprolactone), poly(glycerol monostearate-co-lactide), poly(glycerol monostearate-co-glycolide), poly(glycerol monostearate-co-dioxanone), poly(glycerol monostearate-co-trimethylene carbonate), poly(glycerol monopalmate-co-caprolactone), poly(glycerol monomyristate-co-caprolactone), poly(glycerol monoarachidate-co-caprolactone), poly(glycerol monooleicate-co-caprolactone), poly(glycerol monolinoleicate-co-caprolactone), poly(glycerol monolinoelaidicate-co-caprolactone), and a copolymer or blend thereof.

In another embodiment of this aspect and all other aspects described herein, the composition comprises multiple layers.

In another embodiment of this aspect and all other aspects described herein, the hydrophobic doping agent is selected from the group consisting of a polyester, a polycarbonate, a polyamide, a polyether, a polyanhydride, a copolymer thereof, an oligomer, and a surfactant.

In another embodiment of this aspect and all other aspects described herein, the hydrophobic doping agent is independently selected from the group consisting of poly(caprolactone), poly(lactide-co-glycolide), poly(dioxanone), poly(trimethylene carbonate), poly(ethylene glycol), poly(glycerol monostearate-co-caprolactone), poly(glycerol monostearate-co-lactide), poly(glycerol monostearate-co-glycolide), poly(glycerol monostearate-co-dioxanone), poly(glycerol monostearate-co-trimethylene carbonate), poly(glycerol monopalmate-co-caprolactone), poly(glycerol monomyristate-co-caprolactone), poly(glycerol monoarachidate-co-caprolactone), poly(glycerol monooleicate-co-caprolactone), poly(glycerol monolinoleicate-co-caprolactone), poly(glycerol monolinoelaidicate-co-caprolactone), and a copolymer or blend thereof.

In another embodiment of this aspect and all other aspects described herein, the composition degrades at least 20% slower as compared to the same composition lacking the block co-polymer.

In another embodiment of this aspect and all other aspects described herein, the composition is affixed to the tissue of a subject using a suture, a staple, or an adhesive.

In another embodiment of this aspect and all other aspects described herein, the composition comprises a core-shell structure.

In another embodiment of this aspect and all other aspects described herein, the composition degrades in the range from six to twelve months, inclusive.

In another embodiment of this aspect and all other aspects described herein, the composition degrades in the range from three to six months, inclusive.

In another embodiment of this aspect and all other aspects described herein, the hydrophobic doping agent phase separates within the composition.

In another embodiment of this aspect and all other aspects described herein, the hydrophobic doping agent partitions to the surface of the composition.

In another embodiment of this aspect and all other aspects described herein, the composition comprises an apparent contact angle between 115° and 130°.

In another embodiment of this aspect and all other aspects described herein, the composition comprises an apparent contact angle greater than 130°.

Another aspect described herein relates to the use of the composition as described above as a surgical mesh, a buttressing, a tissue reinforcement, or a tissue scaffolding material.

Another aspect described herein relates to a 3-dimensional composition comprising: a) a biodegradable polymer; b) at least one bioactive agent; and c) a hydrophobic doping agent, wherein the surface hydrophobicity of the composition is substantially homogenous, and wherein the composition comprises entrapped air, wherein the composition is made by the steps of: (a) combining a biodegradable polymer, at least one bioactive agent and a hydrophobic doping agent in an admixture, (b) electrospinning, electrospraying or ultrasonic spraying the admixture, thereby forming the 3-dimensional composition.

Another aspect described herein relates to a method of making a 3-dimensional drug delivery composition, the method comprising the steps of: (a) combining in an admixture a biodegradable polymer, at least one bioactive agent and a hydrophobic doping agent, (b) electrospinning, electrospraying or ultrasonic spraying the admixture, thereby forming the 3-dimensional composition.

In one embodiment of this aspect and all other aspects described herein, the 3-dimensional composition comprises a nanofiber, a microfiber, and/or a bead structure.

In another embodiment of this aspect and all other aspects described herein, the 3-dimensional composition comprises particles or particles fused together.

In another embodiment of this aspect and all other aspects described herein, the composition exhibits tunable drug release via controlled air removal.

In another embodiment of this aspect and all other aspects described herein, the composition comprises between 0.01% and 50% hydrophobic doping agent by weight.

In another embodiment of this aspect and all other aspects described herein, the nanofiber or microfiber comprises a median diameter of between 10 nanometers and 100 micrometers.

In another embodiment of this aspect and all other aspects described herein, the at least one bioactive agent is selected from the group consisting of: an antibiotic, an antimitotic, an anti-inflammatory agent, a growth factor, a targeting compound, a cytokine, an immunotoxin, an anti-tumor antibody, an anti-angiogenic agent, an anti-edema agent, a radiosensitizer and a chemotherapeutic.

In another embodiment of this aspect and all other aspects described herein, the at least one bioactive agent is selected from the group consisting of a taxane, a camptothecin, and a platinum-containing molecule.

In another embodiment of this aspect and all other aspects described herein, the taxane comprises paclitaxel or docetaxol.

In another embodiment of this aspect and all other aspects described herein, the camptothecin comprises irinotecan, topotecan, or SN-38.

In another embodiment of this aspect and all other aspects described herein, the platinum-containing molecule comprises carboplatin or cisplatin.

In another embodiment of this aspect and all other aspects described herein, the biodegradable polymer is selected from the group consisting of a polyester, a polycarbonate, a polyamide, a polyether, a polyanhydride, and a copolymer or blend thereof.

In another embodiment of this aspect and all other aspects described herein, the biodegradable polymer is selected from the group consisting poly(caprolactone), poly(lactide-co-glycolide), poly(dioxanone), poly(trimethylene carbonate), poly(ethylene glycol), poly(glycerol monostearate-co-caprolactone), poly(glycerol monostearate-co-lactide), poly(glycerol monostearate-co-glycolide), poly(glycerol monostearate-co-dioxanone), poly(glycerol monostearate-co-trimethylene carbonate), poly(glycerol monopalmate-co-caprolactone), poly(glycerol monomyristate-co-caprolactone), poly(glycerol monoarachidate-co-caprolactone), poly(glycerol monooleicate-co-caprolactone), poly(glycerol monolinoleicate-co-caprolactone), poly(glycerol monolinoelaidicate-co-caprolactone), and a copolymer or blend thereof.

In another embodiment of this aspect and all other aspects described herein, the composition comprises multiple layers.

In another embodiment of this aspect and all other aspects described herein, the hydrophobic doping agent is selected from the group consisting of a polyester, a polycarbonate, a polyamide, a polyether, a polyanhydride, a copolymer thereof, an oligomer, and a surfactant.

In another embodiment of this aspect and all other aspects described herein, the hydrophobic doping agent is selected from the group consisting of poly(caprolactone), poly(lactide-co-glycolide), poly(dioxanone), poly(trimethylene carbonate), poly(ethylene glycol), poly(glycerol monostearate-co-caprolactone), poly(glycerol monostearate-co-lactide), poly(glycerol monostearate-co-glycolide), poly(glycerol monostearate-co-dioxanone), poly(glycerol monostearate-co-trimethylene carbonate), poly(glycerol monopalmate-co-caprolactone), poly(glycerol monomyristate-co-caprolactone), poly(glycerol monoarachidate-co-caprolactone), poly(glycerol monooleicate-co-caprolactone), poly(glycerol monolinoleicate-co-caprolactone), poly(glycerol monolinoelaidicate-co-caprolactone), and a copolymer or blend thereof.

In another embodiment of this aspect and all other aspects described herein, the composition comprises a core-shell structure.

Another aspect disclosed herein relates to a 3D coating or material comprised of a biodegradable polymer, one or more bioactive agents, and a hydrophobic doping agent, wherein the 3D coating or material delivers one or more bioactive agents for a prolonged time period (e.g., between 1 h and 100 days), wherein an incremental increase in the amount of hydrophobic doping agent content significantly prolongs and/or graduates release of the bioactive agent from the composition.

In one embodiment of this aspect, the hydrophobic doping agent comprises a polymer which is a co-polymer that contains at least a 10 mole % of the base biodegradable polymer and less than 90% of a hydrophobic polymer.

In another embodiment, the hydrophobic doping agent comprises a polymer which is a co-polymer that contains at least a 10 mole % of the base biodegradable polymer and its self is biodegradable.

In another embodiment of this aspect, the hydrophobic doping agent is comprised of an oligomer which contains at least a 10 mole % of the base biodegradable polymer,

In another embodiment of this aspect, the hydrophobic doping agent comprises a small molecule which is hydrophobic.

In another embodiment of this aspect, the inclusion of the hydrophobic doping agent to the polymer at an amount less than or equal to 5 mass percent, reduces the total amount of drug released from the device by at least 20 percent over the first 24 hours of drug release.

In another embodiment of this aspect, the inclusion of the hydrophobic doping agent to the polymer at an amount less than or equal to 5 mass percent, reduces the total amount of drug released from the device by at least 20 percent over the first 10 days of drug release.

In another embodiment of this aspect, the composition comprises between 0.01 and 90 mass percent hydrophobic doping agent, wherein the inclusion of additional hydrophobic doping agent to the polymer at an amount less than or equal to 5 mass percent, reduces the total amount of drug released from the device by at least 20 percent over the first 24 hours of drug release.

In another embodiment of this aspect, the composition comprises between 0.01 and 90 mass percent hydrophobic doping agent, wherein the inclusion of additional hydrophobic doping agent to the polymer at an amount less than or equal to 5 mass percent, reduces the total amount of drug released from the device by at least 20 percent over the first 10 days of drug release.

In another embodiment of this aspect, the inclusion of the hydrophobic doping agent to the polymer at an amount less than or equal to 5 mass percent, increases the contact angle of the coating by at least 10 degrees.

In another embodiment of this aspect, the composition comprises between 5 and 90 mass percent hydrophobic doping agent, wherein the inclusion of additional hydrophobic doping agent to the polymer at an amount less than or equal to 5 mass percent, increases the contact angle of the coating by at least 10 degrees.

In another embodiment of this aspect, the doping agent phase separates within the polymer coating.

In another embodiment of this aspect, the doping agent partitions to the surface of the coating.

In another embodiment of this aspect, the coating is comprised of more than one polymer in addition to the doping agent.

In another embodiment of this aspect, the coating is manufactured by electrospraying, electrospinning, ultrasonic spraying, dip-coating, vapor deposition, spin-coating, knife-coating, melt-coating, or injection molding.

In another embodiment of this aspect, the coating has a porosity of greater than 5% by volume.

In another embodiment of this aspect, the rate of drug release is increased by at least 20% over any 24 hour period when the air content at the surface and/or within the coating is displaced upon exposure to an environmental trigger such as ultrasound, strain, and injection of a surfactant/solvent such as ethanol.

In another embodiment of this aspect, the coating has an apparent contact angle of between 115° and 130°.

In another embodiment of this aspect, the coating has an apparent contact angle of greater than 130°.

In another embodiment of this aspect, surface roughness or texture is added to coating to further increase the apparent contact angle of the coating.

In another embodiment of this aspect, air is maintained at the coating surface and/or within the bulk material for 1 hour to 100 days in an aqueous solution or other liquid.

In another embodiment of this aspect, each of said bioactive agents is independently selected from the group consisting of an antibiotic, an antimitotic, an anti-inflammatory agent, a growth factor, a targeting compound, a cytokine, an immunotoxin, an anti-tumor antibody, an anti-angiogenic agent, an anti-edema agent, a radiosensitizer, and a chemotherapeutic.

In another embodiment of this aspect, each of said bioactive agents is independently selected from the group consisting of a taxane, including paclitaxel and docetaxel, a camptothecin, including irinotecan, topotecan, and SN-38, and a platinum-containing molecule, including carboplatin and cisplatin.

In another embodiment of this aspect, the one or more bioactive agents is/are released from the composition over a time frame effective to inhibit, delay, or prevent tumor growth or inhibit, delay, or prevent metastasis when said coating is affixed nearby, adjacent to, or directly on to the tissue surface at the site of disease.

In another embodiment of this aspect, the one or more bioactive agents is/are released from said coating over a time frame effective to inhibit, delay, or prevent tumor recurrence when said coating is affixed nearby, adjacent to, or directly on to the tumor resection margins following surgery.

In another embodiment of this aspect, bacterial growth and binding is prevented without a bioactive agent.

In another embodiment of this aspect, the loaded bioactive agent is delivered continuously at a therapeutic dose for at least 7 days.

In another embodiment of this aspect, the polymer is independently selected from the group consisting of a polyester, a polycarbonate, a polyamide, a polyether, a polyanhydride, and a copolymer or blend thereof.

In another embodiment of this aspect, the polymer is independently selected from the group consisting of poly(caprolactone), poly(lactide-co-glycolide), poly(dioxanone), poly(trimethylene carbonate), poly(ethylene glycol), poly(glycerol monostearate-co-caprolactone), poly(glycerol monostearate-co-lactide), poly(glycerol monostearate-co-glycolide), poly(glycerol monostearate-co-dioxanone), poly(glycerol monostearate-co-trimethylene carbonate), poly(glycerol monopalmate-co-caprolactone), poly(glycerol monomyristate-co-caprolactone), poly(glycerol monoarachidate-co-caprolactone), poly(glycerol monooleicate-co-caprolactone), poly(glycerol monolinoleicate-co-caprolactone), poly(glycerol monolinoelaidicate-co-caprolactone), and a copolymer or blend thereof.

In another embodiment of this aspect, the composition comprises multiple layers.

In another embodiment of this aspect, the doping agent is independently selected from the group consisting of a polyester, a polycarbonate, a polyamide, a polyether, a polyanhydride, a copolymer thereof, an oligomer, and a surfactant.

In another embodiment of this aspect, the doping agent is independently selected from the group consisting of poly(caprolactone), poly(lactide-co-glycolide), poly(dioxanone), poly(trimethylene carbonate), poly(ethylene glycol), and poly(glycerol monostearate-co-caprolactone), (glycerol monostearate-co-lactide), poly(glycerol monostearate-co-caprolactone), poly(glycerol monostearate-co-lactide), poly(glycerol monostearate-co-glycolide), poly(glycerol monostearate-co-dioxanone), poly(glycerol monostearate-co-trimethylene carbonate), poly(glycerol monopalmate-co-caprolactone), poly(glycerol monomyristate-co-caprolactone), poly(glycerol monoarachidate-co-caprolactone), poly(glycerol monooleicate-co-caprolactone), poly(glycerol monolinoleicate-co-caprolactone), poly(glycerol monolinoelaidicate-co-caprolactone),and a copolymer or blend thereof.

In another embodiment of this aspect, the coating can be applied to a surgical mesh, buttressing, tissue reinforcement, or tissue scaffolding material.

In another embodiment of this aspect, the hydrophobic doping agent is photoactive.

Another aspect described herein relates to a coating or material comprising a biodegradable polymer and a hydrophobic doping agent, which delivers one or more bioactive agents for a prolonged time period, wherein an incremental increase in doping agent content significantly increases the degradation rate of the coating.

In one embodiment of this aspect, the inclusion of the hydrophobic doping agent to the polymer at an amount less than or equal to 5 mass percent, increases the total degradation time of the coating by at least 20 percent.

In another embodiment of this aspect, the composition comprises between 0.01 and 90 mass percent hydrophobic doping agent, wherein the inclusion of additional hydrophobic doping agent to the polymer at an amount less than or equal to 5 mass percent, increases the total degradation time of the coating by at least 20 percent.

In another embodiment of this aspect, the coating or material is prepared via electro spraying or electro spinning.

Another aspect disclosed herein relates to a 3D drug-eluting material comprising: (a) at least one biodegradable polymer, (b) at least one bioactive agent, and (c) entrapped air.

Another aspect disclosed herein relates to a first 3D coating or material composition comprising a biodegradable polymer, at least one bioactive agent, and a hydrophobic doping agent, wherein the first composition releases the at least one bioactive agent for a prolonged period of time compared to a second 3D coating or material composition that lacks or comprises a lower amount of hydrophobic doping agent than the first composition, and wherein an incremental increase in doping agent content in the first composition as compared to the second composition significantly prolongs release of the at least one bioactive agent.

In one embodiment of this aspect, the incremental increase in doping agent content in the first composition as compared to the second composition permits graduated release of the at least one bioactive agent.

In another embodiment of this aspect, the hydrophobic doping agent comprises a co-polymer.

In another embodiment of this aspect, the co-polymer comprises at least a 10 mole % of the base biodegradable polymer and less than 90% of a hydrophobic polymer.

In another embodiment of this aspect, the co-polymer is biodegradable and/or biocompatible.

In another embodiment of this aspect, the hydrophobic doping agent comprises an oligomer and at least a 10 mole % of the at least one biodegradable polymer.

### BRIEF DESCRIPTION OF THE FIGURES

FIG. 1 is a graph that depicts the static contact angle of poly(caprolactone) solvent cast films or electrospun meshes blended with poly(glycerol monostearate-co-e-caprolactone)-poly(caprolactone) (n=4).

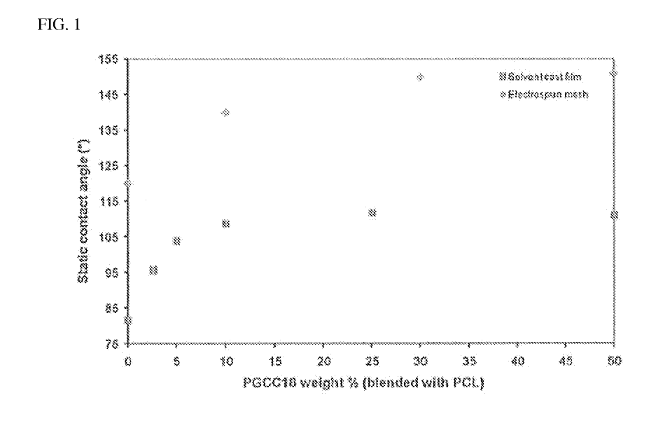

FIG. 2 is a graph that depicts the in vitro drug release of SN38-loaded poly(caprolactone) or poly(glycerol monostearate-co-e-caprolactone)-poly(caprolactone) blended electrospun meshes (n=3).

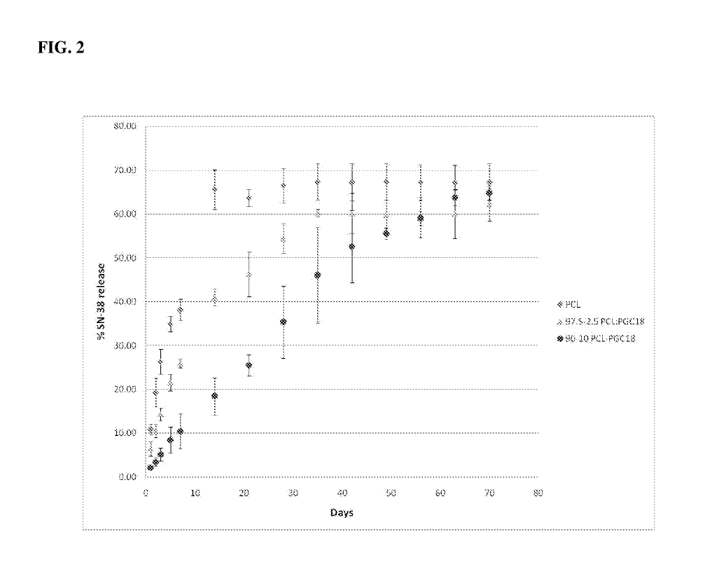

FIG. 3 is a graph that depicts the in vitro drug release of CPT-11-loaded poly(caprolactone) or poly(glycerol monostearate-co-e-caprolactone)-poly(caprolactone) blended electrospun meshes (n=3).

FIG. 4 is a graph that depicts the anti-proliferative effect of high-dose (1% w/w) SN38-loaded poly(caprolactone) or poly(glycerol monostearate-co-e-caprolactone)-poly(caprolactone) blended electrospun meshes on Lewis Lung carcinoma cancer cells.

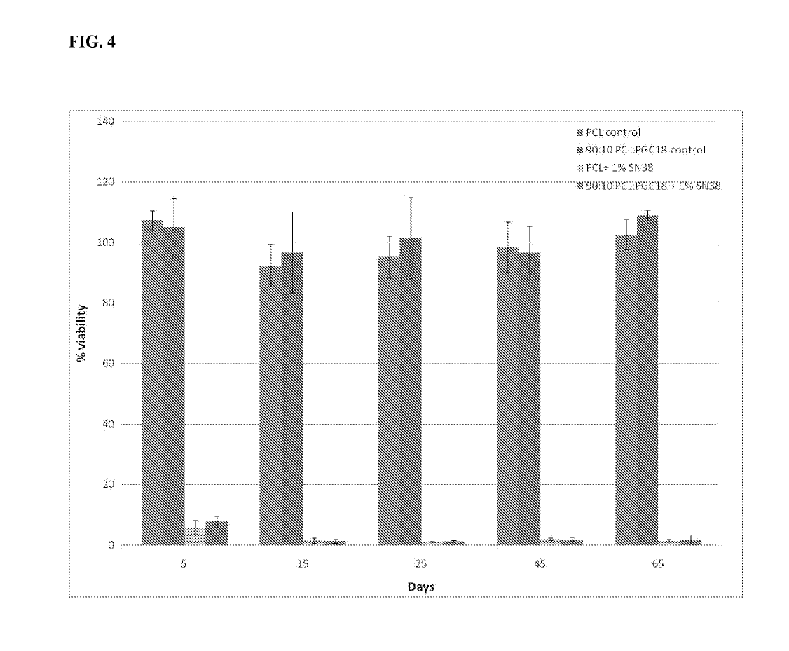

FIG. 5 is a graph that depicts the anti-proliferative effect of medium-dose (0.1% w/w) SN38-loaded poly(caprolactone) or poly(glycerol monostearate-co-e-caprolactone)-poly(caprolactone) blended electrospun meshes on Lewis Lung carcinoma cancer cells.

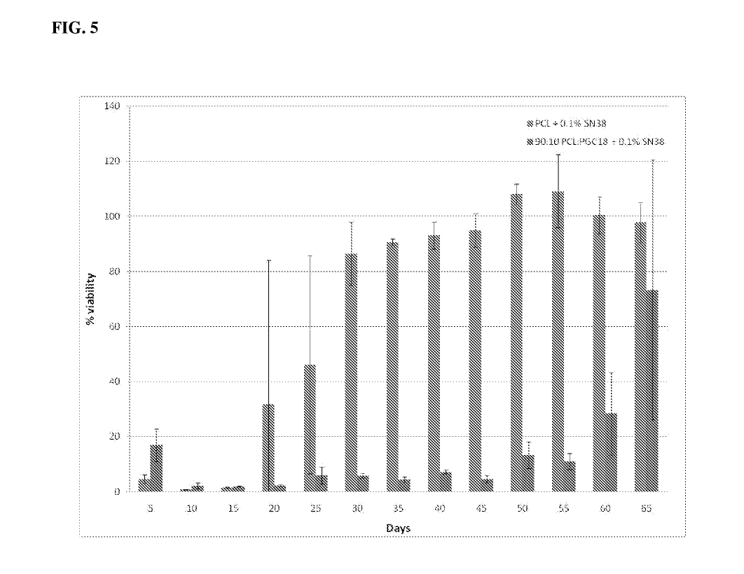

FIG. 6 is a graph that depicts the anti-proliferative effect of low-dose (0.01%) SN38-loaded poly(caprolactone) or poly(glycerol monostearate-co-e-caprolactone)-poly(caprolactone) blended electrospun meshes on Lewis Lung carcinoma cancer cells.

FIG. 7 is a graph that depicts the anti-proliferative effect of high-dose (1% w/w) SN38-loaded poly(caprolactone) or poly(glycerol monostearate-co-e-caprolactone)-poly(caprolactone) blended electrospun meshes on HT-29 colorectal cancer cells.

FIG. 8 is a graph that depicts the anti-proliferative effect of medium- and low-dose (0.1%, 0.01% w/w) SN38-loaded poly(caprolactone) or poly(glycerol monostearate-co-e-caprolactone)-poly(caprolactone) blended electrospun meshes on HT-29 colorectal cancer cells.

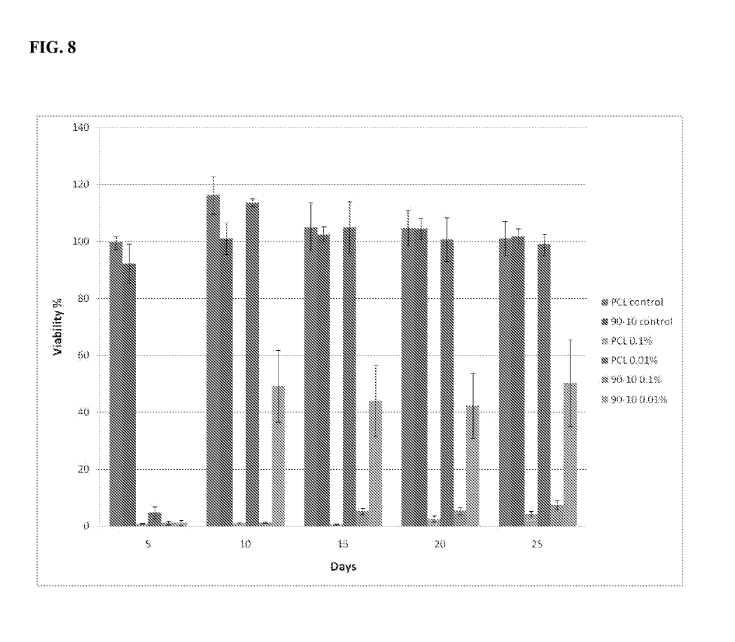

FIG. 9 is a graph that depicts the anti-proliferative effect of high-dose (1%) CPT-11-loaded poly(caprolactone) or poly(glycerol monostearate-co-e-caprolactone)-poly(caprolactone) blended electrospun meshes on HT-29 colorectal cancer cells.

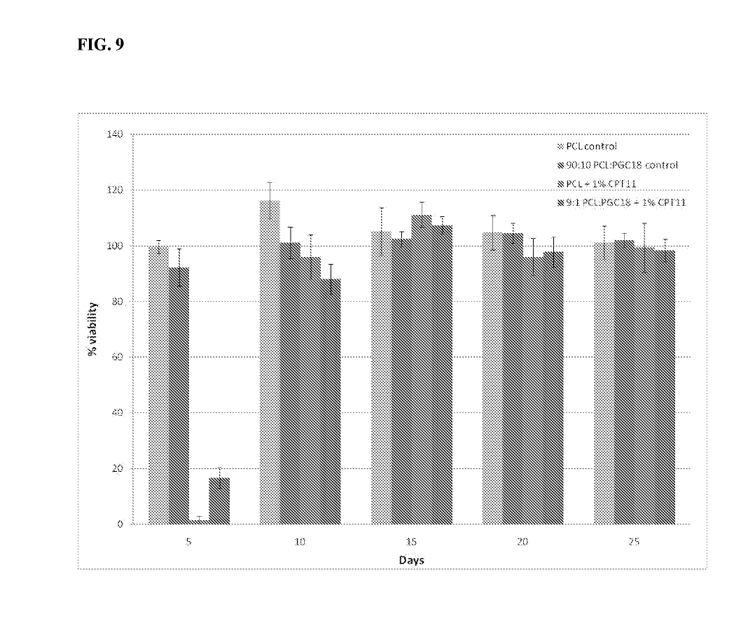

FIGS. 10A-10F depict the following: (FIG. 10A) PCL was used as the base polymer for fabrication of electrospun meshes and melted electrospun meshes. (FIG. 10B) PGC-C18 was used as the hydrophobic dopant in PCL electrospun meshes to decrease the wettability of the meshes. (FIG. 10C) Electrospun PCL mesh with an average fiber size of 7.7±1.2 μm. (FIG. 10D) 10% doped PGC-C18 electrospun PCL mesh with an average fiber size of 7.2±1.4 μm. (FIG. 10E) A melted PCL mesh. (FIG. 10F) A melted 10% doped PGC-C18 electrospun PCL mesh.

FIG. 11 depicts contact angle measurements of poly(glycerol monostearate-co-e-caprolactone)-poly(caprolactone) electrospun meshes and chemically equivalent smooth surfaces as a function of PGC-C18 doping. The black dashed line indicates an approximate boundary for the Wenzel-Cassie state transition.

FIGS. 12A-12B depict release profiles comparing SN-38 release between (FIG. 12A) native, melted and degassed PCL electrospun meshes, and (FIG. 12B) native, melted and degassed 10% PGC-C18 doped PCL electrospun meshes as well as higher PGC-C18 doping concentration of 30 and 50 wt %.

FIGS. 13A-13C depict SN-38 release profiles from electrospun meshes with (FIG. 13A) high (1 wt %), (FIG. 13B) medium (0.1 wt %), and (FIG. 13C) low drug loadings (0.01 wt %). Increasing the percent of PGC-C18 doping into PCL electrospun meshes decreased the release rate of SN-38. Decreasing the drug concentration in electrospun meshes increased the release rate of SN-38.

FIG. 14 depicts release of 1 wt % SN38 from electrospun meshes, with and without 10% PGC-C18 doping, which have been forced to wet with an ethanol dip treatment. PCL meshes and PCL meshes with 10% PGC-C18 release much more quickly than their native, air-containing control. (n=4)

FIG. 15 depicts CT scans of native electrospun and degassed poly(caprolactone) electrospun meshes with 0 or 10% PGC-C18 doping after incubation with the contrast agent Hexabrix™ for 2 hours. Degassed meshes exhibit full water penetration, while native and melted meshes (not shown) show only a low surface concentration of water. Tic marks define the top and bottom boundaries of the meshes.

FIGS. 16A-16B are micrographs depicting representative ultrasound imaging of superhydrophobic electrospun meshes. (FIG. 16A) Image of native electrospun mesh shows a bright hyperechoic surface due to reflectance at the material surface; the rest of the electrospun mesh is dark. (FIG. 16B) Image of degassed electrospun mesh shows full-thickness of electrospun mesh with no air present. Meshes in this study are 300 μm thick.

FIG. 17 is a series of micrographs depicting cross-sectional images of the kinetic infiltration of water into PCL, PCL with 10% PGC-C18, and PCL with 30% PGC-C18 from Day 0 (D0) to as long as 77 days (D77). Water quickly infiltrates into PCL electrospun meshes (unwetted meshes shown in red with increasing water content progressing from yellow to green to blue). Adding 10% PGC-C18 affords a metastable superhydrophobic state, where water slowly infiltrates over time. 30% PGC-C18 achieves a stable superhydrophobic state and water only penetrates the surface of the material. Meshes were 500 μm thick.

FIG. 18 depicts the degree of water infiltration within superhydrophobic electrospun meshes over time. Increasing PGC-C18 and superhydrophobicity leads to slower removal of entrapped air and thus slower wetting. (N=3; Avg±SD)

FIG. 19 depicts an exemplary mechanism of a drug-eluting 3D superhydrophobic material in a metastable Cassie state. Without wishing to be bound by theory, the mechanism indicates that over time water slowly displaces air content from the material with the transition from the metastable Cassie state to the stable Wenzel state. If treated as iterative surfaces, water will slowly penetrate each individual surface over time enabling prolonged drug release.

FIG. 20 depicts micrographs indicating that 3D superhydrophobic materials were produced by electrospinning. By varying the amount of the hydrophobic polymer dopant PGC-C18, the apparent contact angle was increased by virtue of a lower surface energy and higher surface roughness. Spun with 20 wt/v % electrospinning solutions.

FIG. 21 depicts apparent contact angle dependence on electrospun fiber size for four superhydrophobic mesh chemistries. PCL and PCL with 50% PGC-C18 meshes show a continued increase in apparent contact angle with a decrease in fiber size. PCL with 10% PGC-C18 and PCL with 30% PGC-C18 meshes have an initial increase in apparent contact angle, followed by a decrease. Inset shows electrospun PCL with 50% PGC-C18 micro- and nano-fibers, where fiber size is modified through changes in the electrospinning parameters selected. Fibers with diameters below 1500 nm showed a beads-on-a-string morphology. Formulations highlighted in yellow were selected for further study to span a range of superhydrophobicity. (N=3; Avg±SD)

FIG. 22 depicts apparent contact angle measurements for superhydrophobic electrospun meshes when probed with water or water containing SDS. With 0.001 M SDS, PCL meshes did not form an apparent contact angle, and meshes containing 10%, 30%, or 50% PGC-C18 had lower apparent contact angles compared to water alone. Increasing the SDS concentration by 10-fold in the probing solution resulted in no apparent contact angle for all superhydrophobic mesh chemistries other than 50% PGC-C18, where instead a significant drop in apparent contact angle is observed. (N=3; Avg±SD)

FIG. 23 depicts apparent contact angles of superhydrophobic electrospun meshes when (A) probed with polysorbate 20 solutions, or (B) after 24-hour incubation in polysorbate 20 solutions and probed with water. PCL electrospun meshes are completely wetted with polysorbate 20 solution probes and after mesh incubation for all concentrations. In contrast, PCL with 10%, 30%, and 50% PGC-C18 doping only showed a modest decrease in contact angle with polysorbate 20 probes. Incubating 10% and 30% PGC-C18 doped meshes with polysorbate 20 solutions allowed wetting to occur much more readily, with apparent contact angles only observed at the lowest polysorbate 20 concentrations. 50% PGC-C18 meshes are only wetted when incubated with the highest concentration of polysorbate 20. (N=3; Avg±SD)

FIG. 24 depicts the measurement of apparent contact angles using solvents with varied surface tension to determine the critical surface tension required for immediate infiltration into superhydrophobic electrospun meshes. PCL meshes are easily wetted, while increasing PGC-C18 content and superhydrophobicity affords a more robust entrapped air layer in the porous meshes to prevent solvent infiltration. Best fit curves were double exponential. (N=3; Avg±SD)

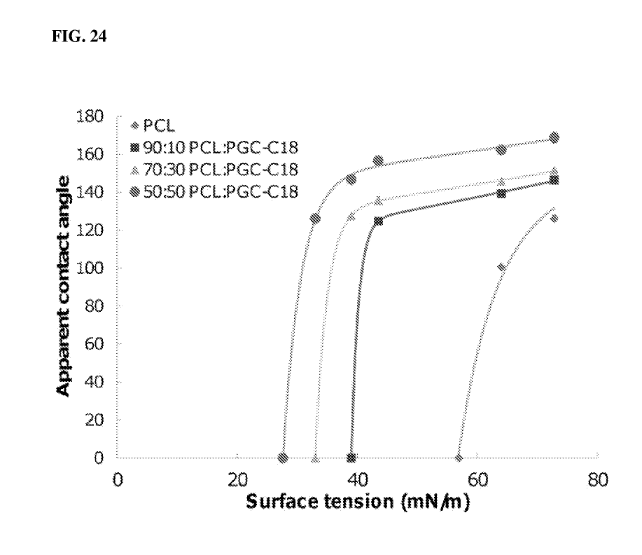

FIG. 25 depicts break through pressures required to force water from the Cassie state to the Wenzel state of wetting. Increasing the PGC-C18 content in PCL meshes increases the amount of pressure necessary to cause water to breakthrough. Meshes using 50% PGC-C18 fractured before infiltration, and could not be used in this study. (N=3; Avg±SD)

FIG. 26 depicts apparent contact angle measurements of superhydrophobic electrospun meshes with and without serum. Meshes were either probed with serum in the applied droplet for contact angle measurements, or were incubated with serum containing solutions for 24 hours, dried, and probed with pure water. A larger decrease in apparent contact angle was seen after incubation in serum for 24 hours, and increasing PGC-C18 content in PCL meshes reduced the decrease in apparent contact angle.

FIGS. 27A-27C depicts HT-29 viability with exposure to electrospun PCL and 10% PGC-C18 doped PCL meshes at three different SN-38 concentrations: (FIG. 27A) 1 wt %, (FIG. 27B) 0.1 wt %, and (FIG. 27C) 0.01 wt %. Unloaded mesh controls were not cytotoxic to cells. No difference in long term cytotoxicity was seen between PCL and 10% PGC-C18 doped PCL with 1 wt % SN-38. Decreasing the SN-38 loading to 0.1 wt % and 0.01 wt % showed superior long term in vitro cytotoxicity with 10% PGC-C18 doped PCL. A Student's t-test was performed at an early, mid, and late timepoint in the assay. An * above a time point signifies p<0.001, whereas a # indicates p>0.01 and is not statistically significant. (n=4)

FIG. 28 depicts HT-29 viability with exposure to 1% CPT-11 loaded PCL and 10% PGC-C18 doped PCL meshes. No difference was seen between PCL and 10% PGC-C18 doped PCL meshes. CPT-11 loaded meshes showed poor long term cancer cell treatment. An * above a time point signifies p<0.001, whereas a # indicates p>0.01 and is not statistically significant. (n=4)

FIGS. 29A-29C depict the following: (FIG. 29A) Synthetic scheme used to produce the hydrophobic polymer dopant PGC-C18 from ε-caprolactone and the carbonate monomer of glycerol. (FIG. 29B) SEM image of a sample electrospun mesh reapproximating around a surgical staple. (FIG. 29C) Sample electrospun strips cut from a larger electrospun mesh which will provide mechanical reinforcement and deliver drug to the colon resection margin.

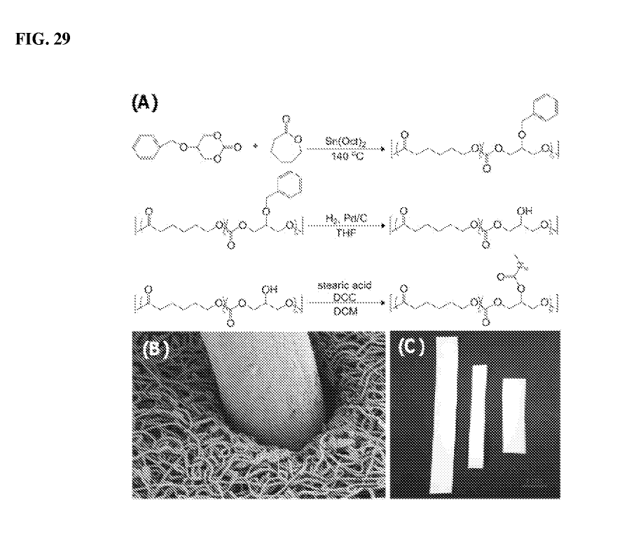

FIG. 30 depicts mechanical performance of PCL and PCL doped with 10% PGC-C18 under constant strain rate. The elastic moduli of electrospun PCL and PCL doped with PGC-C18 are 15.3 and 10.8 MPa, respectively. The ultimate tensile strength of PCL meshes is approximately 1.5 MPa. (n=3)

FIG. 31 depicts the percent of SN-38 released in lactone form from 1 wt % loaded PCL and PCL doped with 10% PGC-C18. Both meshes protect SN-38 from ring opening to the carboxylate form compared to equilibrium values. (n=3)

FIGS. 32A-32C depict the following: (FIG. 32A) An exemplary mechanism of drug release. Air is stable within the superhydrophobic mesh until an ultrasound treatment is used to remove the stable air layer and initiate drug release. (FIG. 32B) Sample PCL electrospun mesh, with fiber sizes of 7.7 μm±1.2. (FIG. 32C) PCL and PGC-C18 were the polymers used to fabricate 3D superhydrophobic meshes.

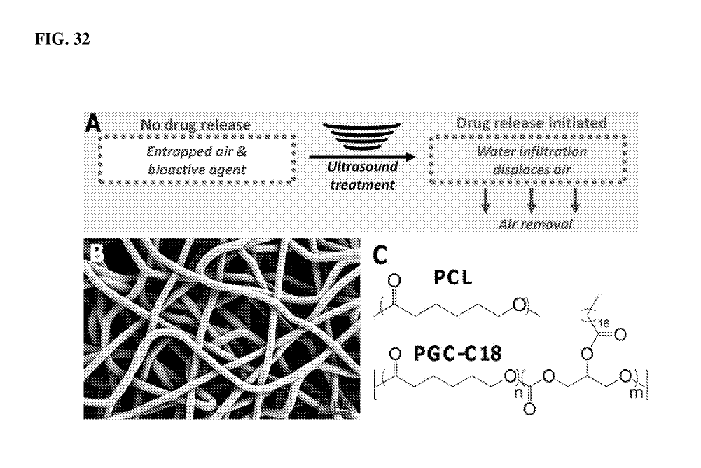

FIGS. 33A-33B depict the following: (FIG. 33A) Photograph of native superhydrophobic electrospun meshes, where with an appropriate HIFU treatment air is removed. Meshes are opaque with air entrapped, and become transparent with water infiltration. (FIG. 33B) Cross section of films in B-mode, showing presence of an air layer within the materials, and removal of the air layer with ultrasound treatment. When the air layer is present (left), the B-mode ultrasound pulses are completely reflected off the surface and the meshes are not visible in the images. When the air layer is removed (right), B-mode ultrasound pulses pass through the surface and the meshes become visible in the images.

FIGS. 34A-34B depicts the wetted area of superhydrophobic meshes as a function of peak rarefaction pressure using (FIG. 34A) continuous wave mode and (FIG. 34B) pulse mode. Statistical significance was tested on three rarefaction pressures. (**, p-value<0.01, PCL→10%, and PCL→30%; *, p-value<0.05, 30%→PCL, and 30%→10%; #, no significance) (n=3; average±SD)

FIGS. 35A-35B depict the following: (FIG. 35A) SN-38 release in PBS (pH 7.4) from PCL with 30% PGC-C18 meshes. An ethanol dip treatment of meshes leads to expeditious release with removal of the air layer. Native, non-degas sed meshes release minimal drug, where ultrasound treatment at day 7 removes the entrapped air layer to initiate release. (FIG. 35B) SN-38 release from PCL with 30% PGC-C18 meshes in PBS supplemented with 10% serum. SN-38 release occurs more quickly than in PBS due to a decrease in surface tension and surfactant binding. Sandwiching the drug-loaded mesh with protective non-drug loaded PCL-30% PGC-C18 layers prevents SN-38 release until initiated by ultrasound treatment at day 7. Drug release eventually occurs with LbL samples which did not receive ultrasound. Arrows indicate time of ultrasound treatment in both plots. Differences in SN-38 release rates from layered meshes before and after ultrasound treatment were statistically significant using analysis of covariance (ANCOVA) (p=0.0012). (n=3; average±SD)

FIG. 36 is a graph depicting data indicating that superhydrophobic meshes without ultrasound treatment are not cytotoxic to cells. After ultrasound treatment at day 10 drug release is initiated and cancer cells are killed. Non-drug loaded meshes are not cytotoxic to cells. (n=3; average±SD) (*=p<0.0001)

FIG. 37 is a series of micrographs depicting SEM images of electrosprayed PCL coating with varying PGC-C18 content (PCL:PGC-C18). Scale bar=20 μm.

FIG. 38 is a graph depicting advancing and receding contact angles of electrosprayed PCL:PGC-C18 blends. (n=3; Avg.±SD)

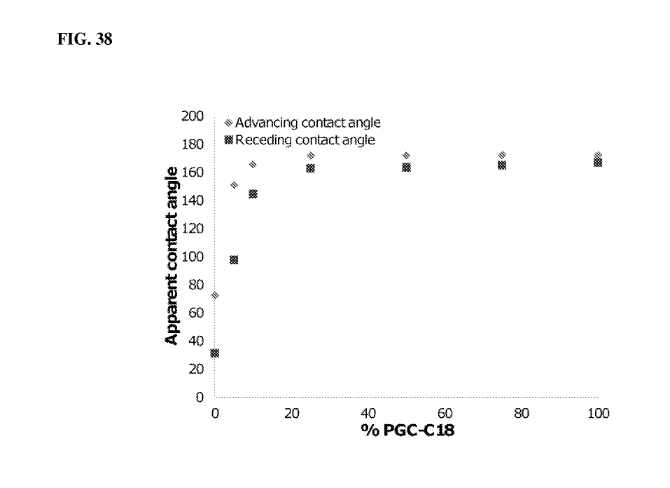

FIG. 39 is a series of micrographs depicting SEM images of electrosprayed coatings produced by varying solution parameters and processing parameters for 75:25 and 50:50 PCL:PGC-C18. All electrosprayed surfaces shown have APA>165°. Scale bar=10 μm.

FIGS. 40A-40B depict the following: (FIG. 40A) Thickness of electrosprayed coatings are controlled by electrospraying deposition time. (n=3; Avg.±SD) (FIG. 40B) μCT cross-sectional images of 73 μm and 156 μm electrosprayed coatings, with and without ethanol treatment. Prior to ethanol treatment and wetting, superhydrophobicity is demonstrated over the entire surface coating and there is no water penetration into the material. After ethanol wetting, water immediately infiltrates into the surface as shown by μCT, where the porosity and the 3D nature of the coatings is confirmed.

FIG. 41 is a series of photographs depicting an electrosprayed superhydrophobic coating on collagen, cotton fabric, nitrile rubber, and aluminum foil. Wettability of the electrosprayed portion of the material (left) is compared to the non-electrosprayed portion (right). Contact angle of all surfaces are >167° with consistent morphology.

FIG. 42 is a graph depicting the release of SN38 as a function of shield/layer/sandwich chemistry in PBS. All meshes had a 90-μm core PCL with SN38 between 150-μm shield layers (n=5 for all conditions, error bars are standard deviations). Black arrows indicate meshes were wetted with ethanol immediately prior to sampling.

FIG. 43 is a graph depicting the release of SN-38 into 10% FBS from meshes with 90-μm PCL core and shield layers of PCL, 10% PCG-18, or 30% PCG-18. Samples from the same meshes used in FIG. 42 were used here.

FIGS. 44A-44C depict the following: (FIG. 44A) Synthetic scheme of PGC-C12-NPE. (FIG. 44B) Photoactive cleavage of NPE group yielding an exposed carboxylic acid and a nitrosoketone byproduct. (FIG. 44C) SEM images of electrospun PCL:PGC-C12-NPE (7:3) meshes (200× magnification (left), 2000× magnification (right))

FIGS. 45A-45B depict the following: (FIG. 45A) UV induced hydrophobicity change from hydrophobic (˜135°) to hydrophilic (˜0°) ACA after 30 minutes of UV exposure. (FIG. 45B) Effects of various UV exposure times (minutes) on the ACA of water (4 μl) on the electrospun polymeric mesh surface over 600 seconds (n=3).

FIG. 46 is a graph depicting NMR evidence of NPE cleavage via diminishing peak integral at ˜6.2 ppm, corresponding to the lone hydrogen on the carbon linking the NPE group to the alkyl chain.

FIG. 47 is a graph depicting three distinct wetting rates as the water droplet infiltrates the photoactive mesh (after 120 minutes of UV exposure)

FIGS. 48A-48B depict the following: (FIG. 48A) μCT imaging of 3D hydrophilic regions within a hydrophobic bulk material using water soluble CT contrast agent penetration into the meshes. (FIG. 48B) CT contrast agent penetration versus UV exposure time. (n=3)

### DETAILED DESCRIPTION

Described herein are 3-dimensional drug-eluting materials comprising biodegradable polymer(s), one or more bioactive agents (e.g., drug(s)) and entrapped air. Various embodiments of the methods and compositions described herein are based, in part, on the discovery of hydrophobic doping agents that can be used in the manufacture of polymeric drug delivery compositions that permit the encapsulation of air, thereby permitting tunable drug release via controlled air removal. Such 3-dimensional compositions can comprise, for example, a) a biodegradable polymer and a hydrophobic doping agent used to create entrapped air, and b) a bioactive agent embedded in the polymer. Such compositions are particularly useful for delivering therapeutically effective doses of one or more bioactive agents to a subject over an extended period of time (e.g., days, weeks, or months).

### Definitions

As used herein, the term “bioactive agent” refers to an agent that is capable of exerting a biological effect in vitro and/or in vivo. The biological effect can be therapeutic in nature. As used herein, “bioactive agent” refers also to a substance that is used in connection with an application that is diagnostic in nature, such as in methods for diagnosing the presence or absence of a disease in a patient. The bioactive agents can be neutral or positively or negatively charged. Examples of suitable bioactive agents include pharmaceuticals and drugs, cells, gases and gaseous precursors (e.g., O₂), synthetic organic molecules, proteins, enzymes, growth factors, vitamins, steroids, polyanions, nucleosides, nucleotides, polynucleotides, and diagnostic agents, such as contrast agents for use in connection with magnetic resonance imaging, ultrasound, positron emission transmography, computed tomography, or other imaging modality of a patient.

As used herein, the term “biocompatible” refers to the absence of an adverse acute, chronic, or escalating biological response to an implant or coating, and is distinguished from a mild, transient inflammation which typically accompanies surgery or implantation of foreign objects into a living organism.

As used herein, the term “biodegradable” refers to the erosion or degradation of a material into smaller entities which will be metabolized or excreted under the conditions normally present in a living tissue. Biodegradation is preferably predictable both in terms of the degradation products formed, including metabolic byproducts formed, and in terms of duration, whereas the duration of biodegradation can be dependant upon the chemical structure of the material.

As used herein, the terms “controlled release,” “sustained release,” and “prolonged release” refer to the continuous release of drugs from a material for at least 24 hours wherein the release can be substantially constant or vary as a function of time. In some embodiments, the continuous release is greater than 30 days. In some embodiments, the release kinetics are linear and repeatable.

As used herein, the terms “compliance” or “compliant” are used in a general sense and refer, for example, to the ability of an implant to closely match the mechanical properties of tissues at the implant site, such as in the sense of bending or flexing with the natural movement of tissues at the implant site, except when “compliance” is used in the specific technical sense as the reciprocal of modulus.

As used herein, the term “doping agent” refers to a polymer, oligomer, or a small molecule that is incorporated into a primary polymer composition for the purpose of altering one or more implant properties, including, but not limited to, wet-ability, hydrophobicity, drug release kinetics, degradation profile, biocompatibility, and/or mechanical compliance. A hydrophobic doping agent refers to a doping agent that is hydrophobic. As used herein, the term “doping” is a verb meaning “to dope” or “add in a doping agent.” In one embodiment, a hydrophobic doping agent comprises a co-polymer comprising a composition of the base (main) polymer or one of similar chemical structure and a second component (e.g., a polycarbonate of glycerol modified with a long chain fatty acid). In one embodiment, the hydrophobic doping agent comprises a “block co-polymer.”

As used herein, the term “co-polymer” refers to a polymer comprised of at least two different monomer constituents. A copolymer can comprise a base (main) monomer (which forms a biodegradable polymer) is polymerized with a doping agent as described herein. In some embodiments, a co-polymer including doping agent in this manner is prepared and then mixed with the biodegradable polymer (i.e., the first monomer polymerized without the doping agent) and bioactive agent in the manufacture of a 3-dimensional composition as described herein. The co-polymer can comprise a block co-polymer or random co-polymer structure.

As used herein, the term “pharmaceutical composition” refers to a chemical compound or composition capable of inducing a desired therapeutic effect in a subject. In certain embodiments, a pharmaceutical composition contains an active agent, which is the agent that induces the desired therapeutic effect. The pharmaceutical composition can contain a prodrug of the compounds provided herein. In certain embodiments, a pharmaceutical composition contains inactive ingredients, such as, for example, carriers and excipients.

As used herein, the term “pharmaceutically acceptable” refers to a formulation of a compound that does not significantly abrogate the biological activity, a pharmacological activity and/or other properties of the compound when the formulated compound is administered to a subject. In certain embodiments, a pharmaceutically acceptable formulation does not cause significant irritation to a subject.

As used herein, pharmaceutically acceptable derivatives of a compound include, but are not limited to, salts, esters, enol ethers, enol esters, acetals, ketals, orthoesters, hemiacetals, hemiketals, acids, bases, solvates, hydrates, PEGylation, or prodrugs thereof. Such derivatives can be readily prepared by those of skill in this art using known methods for such derivatization. The compounds produced can be administered to animals or humans without substantial toxic effects and either are pharmaceutically active or are prodrugs. Pharmaceutically acceptable salts include, but are not limited to, amine salts, such as but not limited to chloroprocaine, choline, N,N′-dibenzyl-ethylenediamine, ammonia, diethanolamine and other hydroxyalkylamines, ethylenediamine, N-methylglucamine, procaine, N-benzyl-phenethylamine, 1-para-chloro-benzyl-2-pyrrolidin-1′-ylmethyl-benzimidazole, diethylamine and other alkylamines, piperazine and tris(hydroxymethyl)-aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates. Pharmaceutically acceptable esters include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl, aryl, heteroaryl, aralkyl, heteroaralkyl, cycloalkyl, or heterocyclyl. Pharmaceutically acceptable enol esters include, but are not limited to, derivatives of formula C═C(OC(O)R) where R is hydrogen, alkyl, alkenyl, alkynyl, aryl, heteroaryl, aralkyl, heteroaralkyl, cycloalkyl, or heterocyclyl. Pharmaceutically acceptable solvates and hydrates are complexes of a compound with one or more solvent or water molecules, or 1 to about 100, or 1 to about 10, or one to about 2, 3, or 4, solvent or water molecules.

As used herein, the term “subject” refers to a human or an animal, typically a mammal, such as a cow, horse, dog, cat, pig, sheep, monkey, or other laboratory or domesticated animal. As used herein, the term “patient” includes human and animal subjects.

The phrase “therapeutically effective amount” refers to the amount of a pharmaceutical composition that elicits the biological or medicinal response in a tissue, system, animal, individual, patient, or human that is being sought by a researcher, veterinarian, medical doctor or other clinician.

As used herein, the terms “treating” or “treatment” encompass either or both responsive and prophylaxis measures, e.g., designed to inhibit, slow, or delay the onset of a symptom of a disease or disorder, achieve at least a partial reduction of a symptom or disease state, and/or to alleviate, ameliorate, lessen, or cure a disease or disorder and/or its symptoms.

As used herein, “tunable drug release” refers to the ability to reduce either the cumulative amount of released drug over a fixed time period by at least 20 percent, or the ability to alter the rate of drug release over a fixed time period by at least 20 percent, or both.

As used herein, amelioration of the symptoms of a particular disorder by administration of a particular compound or pharmaceutical composition refers to any lessening of severity, delay in onset, slowing of progression, or shortening of duration, whether permanent or temporary, lasting or transient that can be attributed to or associated with administration of the compound or composition.

Meshes can be used with the methods and compositions described herein and include commercially available products. Examples of films and meshes include INTERCEED (Johnson & Johnson, Inc.), PRECLUDE (W.L. Gore), and POLYACTIVE (poly(ether ester) multiblock copolymers (Osteotech, Inc., Shrewsbury, N.J.), based on poly(ethylene glycol) and poly(butylene terephthalate), and SURGICAL absorbable hemostat gauze-like sheet from Johnson & Johnson. Another mesh is a prosthetic polypropylene mesh with a bioresorbable coating called SEPRAMESH Biosurgical Composite (Genzyme Corporation, Cambridge, Mass.). One side of the mesh is coated with a bioresorbable layer of sodium hyaluronate and carboxymethylcellulose, providing a temporary physical barrier that separates the underlying tissue and organ surfaces from the mesh. The other side of the mesh is uncoated, allowing for complete tissue ingrowth similar to bare polypropylene mesh. Other films and meshes include: (a) BARD MARLEX mesh (C.R. Bard, Inc.), which has a very dense knitted fabric structure with low porosity; (b) monofilament polypropylene mesh such as PROLENE available from Ethicon, Inc. Somerville, N.J. (see, e.g., U.S. Pat. Nos. 5,634,931 and 5,824,082)); (c) SURGISIS GOLD and SURGISIS IHM soft tissue graft (both from Cook Surgical, Inc.) which are devices specifically configured for use to reinforce soft tissue in repair of inguinal hernias in open and laparoscopic procedures; (d) thin walled polypropylene surgical meshes such as are available from Atrium Medical Corporation (Hudson, N.H.) under the trade names PROLITE, PROLITE ULTRA, and LITEMESH; (e) COMPOSIX hernia mesh (C.R. Bard, Murray Hill, N.J.), which incorporates a mesh patch (the patch includes two layers of an inert synthetic mesh, generally made of polypropylene, and is described in U.S. Pat. No. 6,280,453) that includes a filament to stiffen and maintain the device in a flat configuration; (f) VISILEX mesh (from C.R. Bard, Inc.), which is a polypropylene mesh that is constructed with monofilament polypropylene; (g) other meshes available from C.R. Bard, Inc. which include PERFIX Plug, KUGEL Hernia Patch, 3D MAX mesh, LHI mesh, DULEX mesh, and the VENTRALEX Hernia Patch; and (h) other types of polypropylene monofilament hernia mesh and plug products include HERTRA mesh 1, 2, and 2A, HERMESH 3, 4 & 5 and HERNIAMESH plugs T1, T2, and T3 from Herniamesh USA, Inc. (Great Neck, N.Y.).

Where reference is made to a URL or other such identifier or address, it understood that such identifiers can change and particular information on the internet can come and go, but equivalent information can be found by searching the internet. Reference thereto evidences the availability and public dissemination of such information.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

### Polymer Carriers and Composition

Superhydrophobic surfaces are defined by large apparent contact angles with water that traditionally exceed 150° (Ma, M. and Hill, R. M. Current Opinion in Colloid & Interface Science (2006) 11, 193; Roach, P. et al., Soft Matter (2008) 4, 224; Nakajima, A. et al., Chem. (2001) 132, 31; Rothstein, J. P. Annual Review of Fluid Mechanics (2010) 42, 89; Lafuma, A. and Quere, D. Nat. Mater. (2003) 2, 457; Patankar, N. and A. Langmuir (2004) 20, 7097; Wang, S. and Jiang, L. Adv. Mater. (2007) 19, 3423). These surfaces are found in nature on animal fur, plant leaves, and insect legs, and are now commonly produced synthetically by adding surface roughness to a low surface energy material using a variety of processing techniques (Li, X.-M. et al., Chem. Soc. Rev. (2007) 36, 1350; Xue, C. H. et al., Science and Technology of Advanced Materials (2010) 11, 033002; Yan, Y. et al., Advances in colloid and interface science (2011) 169, 80; Feng, X. and Jiang, L. Advanced Materials (2006) 18, 3063; Ma, M. et al., J. Adhes. Sci. Technol. (2008) 22, 1799; Levkin, P. A. et al., J. Advanced functional materials (2009) 19, 1993; Tuteja, A. et al., Science (2007) 318, 1618). The superhydrophobic nature of these surfaces is realized when air is maintained at the water-material surface to produce a more energetically favorable interface. This “trapped” air leads to the characteristically high apparent contact angles of superhydrophobic surfaces with a composite air-material surface maintained under the water droplets.

Superhydrophobic surfaces submerged in water are being investigated to determine whether the air layer at the material surface is stable over extended periods. In these underwater stability assays, a layer of water is applied to test the superhydrophobic surface. Several theoretical studies have described the permanent stability of air at superhydrophobic surfaces, and with an appropriate surface roughness, chemistry, geometry, and aspect ratio, the air layer is predicted to be maintained indefinitely (Marmur, A. Langmuir (2006) 22, 1400; Fukagata, K. et al., Physics of Fluids (2006), 18, 089901/1). The long-term underwater stability of air at superhydrophobic surfaces has also been demonstrated empirically with examples from natural and engineered surfaces. Some of these materials are able to maintain their superhydrophobic properties despite more stringent degassing conditions, including turbulent flow, surfactant addition, and increased water immersion depth (Bobji, M. S. et al., Langmuir (2009) 25, 12120; Poetes, R. et al., Physical Review Letters (2010) 105, 166104/1; McHale, G. et l., Soft Matter (2010) 6, 714; Ou, J. and Rothstein, J. P. Physics of Fluids (2005) 17, 103606/1; Truesdell, R. et al., Physical Review Letters (2006) 97, 044504/1; Gao, X. and Jiang, L. Nature (2004) 432, 36; Shirtcliffe, N. J.; et al., Applied Physics Letters (2006) 89, 104106/1; Liu, T. et al., Electrochimica Acta (2007) 52, 3709; Zhang, H. et al., Science and Technology of Advanced Materials (2005) 6, 236). The maintenance of air at the water-material interface can lead to improved performance, and superhydrophobic surfaces are being evaluated for applications in drag reduction, corrosion prevention, reduction of biofouling, improving buoyancy, self-cleaning, etc. (Nosonovsky, M. and Bhushan, B. Current Opinion in Colloid & Interface Science (2009) 14, 270; Zhang, X. et al., J. Mater. Chem. (2008) 18, 621; Genzer, J. and Efimenko, K. Biofouling (2006) 22, 339; Marmur, A. Biofouling (2006) 22, 107; Yao, X. et al., Advanced Materials (2011) 23, 719; Voronov, R. S. et al., Industrial & Engineering Chemistry Research (2008) 47, 2455; Plawsky, J. L. et al., Chemical Engineering Communications (2008) 196, 658; Verplanck, N. et al., Nanoscale Research Letters (2007), 2, 577; Heikenfeld, J. and Dhindsa, M. Journal of Adhesion Science and Technology, 22 (2008) 3, 319).

In some embodiments, a composition is disclosed herein for delivery of one or more bioactive agents, the composition comprising a bioactive agent, a biodegradable and/or biocompatible polymer (e.g., polymer carrier), and a hydrophobic doping agent (e.g., polymers, oligomers, or small molecules). In some embodiments, the incorporation of the hydrophobic doping agent into the polymer carrier imparts an effect on the release kinetics of the embedded bioactive agent and/or imparts an effect on the degradation rate of the polymer carrier.

In some embodiments, the composition comprises a non-woven mesh form factor with an average thickness between 0.5 to 1000 μm. The fibers comprising the mesh can have an average diameter between 10 nm to 100 μm. In some embodiments, the device is comprised of multiple layers of mesh, in which one or more layers contain a therapeutic agent. In some embodiments, the composition can be biocompatible, biodegradable, and/or composed of natural or synthetic polymers capable of conforming to irregular tissue surfaces. The mesh material can be relatively thin compared to the tissue, and meet the appropriate mechanical requirements, such as compliance, which can be achieved, but is not limited to the selection of compliant polymers, or the processing of otherwise rigid polymers into a flexible state, i.e., a knitted or woven network of fibers like that found in DACRON® vascular prostheses. In some embodiments, the mesh material can have sufficient mechanical properties to be utilized in buttressing indications, such as following a surgical resection to prevent tears or leaks in weakened tissue at or near the resection margins. Such buttressing materials are usually stapled into place using a surgical stapler that simultaneously cuts as it staples, or the materials are sutured into place. A buttressing or mesh material can be administered to tissue in any manner that ensures the scaffold is affixed in place.

In some embodiments, the compositions described herein comprise one or more materials independently selected from the group consisting of a polyester, a polycarbonate, a polyamide, a polyether, a polyanhydride, a copolymer thereof, collagen, modified collagen, hylauronic acid, and a natural polymer. In some embodiments, the composition comprises one or more materials independently selected from the group consisting of poly(caprolactone), polylactide, polyglycolide, poly(lactide-co-glycolide), poly(dioxanone), poly(trimethylene carbonate), poly(ethylene glycol), and poly(glycerol monostearate-co-caprolactone) poly(glycerol monostearate-co-lactide), poly(glycerol monostearate-co-glycolide), poly(glycerol monostearate-co-dioxanone), poly(glycerol monostearate-co-trimethylene carbonate), poly(glycerol monopalmate-co-caprolactone), poly(glycerol monomyristate-co-caprolactone), poly(glycerol monoarachidate-co-caprolactone), poly(glycerol monooleicate-co-caprolactone), poly(glycerol monolinoleicate-co-caprolactone), poly(glycerol monolinoelaidicate-co-caprolactone), and a copolymer or blend thereof.

In certain embodiments, the compositions as described herein comprise at least one superhydrophobic surface (e.g., 2, 3, 4, 5, 10, 20, 30, 40, 50, 100, 200, 500 etc.), wherein the surface exhibits an apparent water contact angle of at least 115°; in other embodiments, the composition comprises a water contact angle of at least 120°, at least 130°, at least 140°, at least 150 °, at least 155°, at least 160°, at least 165°, at least 170°, at least 175° or more. In some embodiments, the composition has an apparent contact angle of between 115° and 130°, between 120° and 130°, between 125° and 130°, between 115° and 120°, between 115° and 125°, or between 120° and 125°. In one embodiment, the compositions as described herein comprise surface properties that are substantially homogeneous, for example, a material having substantially homogeneous properties (e.g., a consistent contact angle) e.g., throughout the bulk of the composition. It is important to note that the property of homogeneity refers to a surface characteristic (e.g., water contact angle) and not to the consistency of the composition.

In one embodiment, surface roughness or texture is added to the composition to further increase the apparent contact angle of the composition.

### Hydrophobic Doping Agents

A hydrophobic doping agent as that term is used herein permits the preparation of a 3-dimensional drug delivery composition with superior properties, e.g., a longer release time of a bioactive agent. A hydrophobic doping agent can comprise a co-polymer, a hydrophobic small molecule or an oligomer. Hydrophobic doping of a polymer in the making of a composition is a generic effect where separate homopolymers/co-polymer systems have the same effect. Thus, one of skill in the art can successfully design a number of polymeric drug compositions by varying the polymer and hydrophobic doping agent combinations.

In one embodiment, the hydrophobic doping agent is biodegradable. In another embodiment, the hydrophobic doping agent is biocompatible.

An advantage of employing a hydrophobic doping agent is that one can add small amounts of the dopant and achieve very large contact angles. Thus, it is not necessary to blend large quantities of polymers to achieve a composition that is capable of delivering a bioactive agent to a subject.

Without wishing to be bound by theory, the most pronounced effect observed with an increase in hydrophobic doping agent when electrospinning is an increase in fiber roughness (pores or ripples on each fiber) and/or a decrease in fiber size. When electrospraying, this translates to a high particle roughness (pores or ripples on each particle) and/or a decrease in particle size. These changes lead to a higher material porosity, and in turn more air entrapped within the same material volume. These effects may be due both to the difference in chemistry between the polymer and the hydrophobic dopant, leading to potential phase separation at the surface and within the bulk material, and/or a lower molecular weight of the dopant.

In one embodiment, the hydrophobic doping agent comprises a co-polymer. In another embodiment, the hydrophobic doping agent comprises a block co-polymer. In one such embodiment, the co-polymer comprises a composition of the base (main) polymer, or one of similar chemical structure, and a second component (e.g., a polycarbonate of glycerol modified with a long chain fatty acid). The fatty acid can be saturated or unsaturated.

In one embodiment, the hydrophobic doping agent comprises a co-polymer comprising at least 10 mole % of the base biodegradable polymer and less than 90% of a hydrophobic polymer. In other embodiments, the hydrophobic doping agent comprises a co-polymer comprising at least 5 mole % of the base biodegradable polymer and less than 95% of a hydrophobic polymer, or at least 15 mole % of the base biodegradable polymer and less than 85% of a hydrophobic polymer, or at least 20 mole % of the base biodegradable polymer and less than 80% of a hydrophobic polymer, or at least 25 mole % of the base biodegradable polymer and less than 75% of a hydrophobic polymer, or at least 30 mole % of the base biodegradable polymer and less than 70% of a hydrophobic polymer.

In another embodiment, the hydrophobic doping agent comprises an oligomer. In such embodiments, the hydrophobic doping agent comprises at least 10% mole of the base biodegradable polymer in combination with an oligomer; in other embodiments, the hydrophobic doping agent comprises an oligomer in combination with at least 5 mole % of the base polymer, at least 15 mole % of the base polymer, at least 20 mole % of the base polymer, at least 25 mole % of the base polymer, at least 30 mole % of the base polymer or more.

In one embodiment, inclusion of the hydrophobic doping agent to the polymer in an amount less than or equal to 5 mass percent, reduces the total amount of drug released from the device by at least 20% over the first 24 hours of drug release. That is, an incremental increase in hydrophobic doping agent results in a significant decrease in drug release rate. By “significant decrease” in this context is meant that the effect of incrementally increasing hydrophobic doping agent modifies release characteristics in a non-incremental or at least non-linear manner. In another embodiment, inclusion of the hydrophobic doping agent to the polymer in an amount less than or equal to 5 mass percent, reduces the total amount of drug released from the device by at least 20% over the first 10 days of drug release. In another embodiment, the composition as described herein comprises between 0.01 and 90 mass percent hydrophobic doping agent, wherein the inclusion of additional hydrophobic doping agent to the polymer in an amount less than or equal to 5 mass percent, reduces the total amount of drug released from the device by at least 20% over the first 24 hours of drug release. In another embodiment, the composition as described herein comprises between 0.01 and 90 mass percent hydrophobic doping agent, wherein the inclusion of additional hydrophobic doping agent to the polymer in an amount less than or equal to 5 mass percent, reduces the total amount of drug released from the device by at least 20% over the first 10 days of drug release.

In another embodiment, the inclusion of the hydrophobic doping agent to the polymer at an amount less than or equal to 5 mass percent increases the contact angle of the 3-dimensional composition by at least 10 degrees (e.g., at least 15, at least 20, at least 25, at least 30, at least 35, at least 40, at least 45, at least 50 degrees or more). In another embodiment, the composition comprises between 5 and 90 mass percent hydrophobic doping agent, wherein the inclusion of additional hydrophobic doping agent to the polymer at an amount less than or equal to 5 mass percent increases the contact angle of the coating by at least 10 degrees.

In some embodiments, the hydrophobic doping agent phase separates within the polymer coating during the manufacture of the 3-dimensional composition by e.g., electrospinning, electrospraying, or ultrasonic spraying. In other embodiments, the hydrophobic doping agent partitions to the surface of the coating during the manufacture of the 3-dimensional composition by e.g., electrospinning or electrospray.

In one embodiment, the hydrophobic doping agent is photoactive.

In one embodiment, the hydrophobic doping agent is not a perfluorocarbon polymer or compound (e.g., poly(perfluoroalkyl ethyl methacrylate (PPFEMA)).

### Agents to be Encapsulated

The compositions provided herein can be used to deliver any bioactive agent. The agent can be in any pharmaceutically acceptable form, including pharmaceutically acceptable salts. A large number of pharmaceutical agents are known in the art and are amenable for use in the pharmaceutical compositions of the polymeric materials described herein. Acceptable agents include, but are not limited to, chemotherapeutic agents, such as radiosensitizers, receptor inhibitors and agonists or other anti-neoplastic agents; immune modulators and bioactive agents, such as cytokines, growth factors, or steroids with or without the co-incorporation of tumor or pathogen antigens to increase the anti-neoplastic response as a means of vaccine development; local anesthetic agents; antibiotics; or nucleic acids as a means of local gene therapy.

Any agent can be incorporated within the polymer films and particles described herein. For example, a polymer mesh described herein can incorporate a pharmaceutical agent selected from among (1) nonsteroidal anti-inflammatory drugs (NSAIDs) analgesics, such as diclofenac, ibuprofen, ketoprofen, and naproxen; (2) opiate agonist analgesics, such as codeine, fentanyl, hydromorphone, and morphine; (3) salicylate analgesics, such as aspirin (ASA) (enteric coated ASA); (4) H1-blocker antihistamines, such as clemastine and terfenadine; (5) H2-blocker antihistamines, such as cimetidine, famotidine, nizadine, and ranitidine; (6) anti-infective agents, such as mupirocin; (7) anti-anaerobic anti-infectives, such as chloramphenicol and clindamycin; (8) antifungal antibiotic anti-infectives, such as amphotericin b, clotrimazole, fluconazole, and ketoconazole; (9) macrolide antibiotic anti-infectives, such as azithromycin and erythromycin; (10) miscellaneous beta-lactam antibiotic anti-infectives, such as aztreonam and imipenem; (11) penicillin antibiotic anti-infectives, such as nafcillin, oxacillin, penicillin G, and penicillin V; (12) quinolone antibiotic anti-infectives, such as ciprofloxacin and norfloxacin; (13) tetracycline antibiotic anti-infectives, such as doxycycline, minocycline, and tetracycline; (14) antituberculosis antimycobacterial anti-infectives such as isoniazid (INH), and rifampin; (15) antiprotozoal anti-infectives, such as atovaquone and dapsone; (16) antimalarial antiprotozoal anti-infectives, such as chloroquine and pyrimethamine; (17) anti-retroviral anti-infectives, such as ritonavir and zidovudine; (18) antiviral anti-infective agents, such as acyclovir, ganciclovir, interferon alpha, and rimantadine; (19) alkylating antineoplastic agents, such as carboplatin and cisplatin; (20) nitrosourea alkylating antineoplastic agents, such as carmustine (BCNU); (21) antimetabolite antineoplastic agents, such as methotrexate; (22) pyrimidine analog antimetabolite antineoplastic agents, such as fluorouracil (5-FU) and gemcitabine; (23) hormonal antineoplastics, such as goserelin, leuprolide, and tamoxifen; (24) natural antineoplastics, such as aldesleukin, interleukin-2, docetaxel, etoposide (VP-16), interferon alpha, paclitaxel, and tretinoin (ATRA); (25) antibiotic natural antineoplastics, such as bleomycin, dactinomycin, daunorubicin, doxorubicin, and mitomycin; (26) vinca alkaloid natural antineoplastics, such as vinblastine and vincristine; (27) autonomic agents, such as nicotine; (28) anticholinergic autonomic agents, such as benztropine and trihexyphenidyl; (29) antimuscarinic anticholinergic autonomic agents, such as atropine and oxybutynin; (30) ergot alkaloid autonomic agents, such as bromocriptine; (31) cholinergic agonist parasympathomimetics, such as pilocarpine; (32) cholinesterase inhibitor parasympathomimetics, such as pyridostigmine; (33) alpha-blocker sympatholytics, such as prazosin; (34) beta-blocker sympatholytics, such as atenolol; (35) adrenergic agonist sympathomimetics, such as albuterol and dobutamine; (36) cardiovascular agents, such as aspirin (ASA), plavix (Clopidogrel bisulfate) etc; (37) beta-blocker antianginals, such as atenolol and propranolol; (38) calcium-channel blocker antianginals, such as nifedipine and verapamil; (39) nitrate antianginals, such as isosorbide dinitrate (ISDN); (40) cardiac glycoside antiarrhythmics, such as digoxin; (41) class I anti-arrhythmics, such as lidocaine, mexiletine, phenytoin, procainamide, and quinidine; (42) class II antiarrhythmics, such as atenolol, metoprolol, propranolol, and timolol; (43) class III antiarrhythmics, such as amiodarone; (44) class IV antiarrhythmics, such as diltiazem and verapamil; (45) alpha-blocker antihypertensives, such as prazosin; (46) angiotensin-converting enzyme inhibitor (ACE inhibitor) antihypertensives, such as captopril and enalapril; (47) beta blocker antihypertensives, such as atenolol, metoprolol, nadolol, and propanolol; (48) calcium-channel blocker antihypertensive agents, such as diltiazem and nifedipine; (49) central-acting adrenergic antihypertensives, such as clonidine and methyldopa; (50) diurectic antihypertensive agents, such as amiloride, furosemide, hydrochlorothiazide (HCTZ), and spironolactone; (51) peripheral vasodilator antihypertensives, such as hydralazine and minoxidil; (52) antilipemics, such as gemfibrozil and probucol; (53) bile acid sequestrant antilipemics, such as cholestyramine; (54) HMG-CoA reductase inhibitor antilipemics, such as lovastatin and pravastatin; (55) inotropes, such as amrinone, dobutamine, and dopamine; (56) cardiac glycoside inotropes, such as digoxin; (57) thrombolytic agents or enzymes, such as alteplase (TPA), anistreplase, streptokinase, and urokinase; (58) dermatological agents, such as colchicine, isotretinoin, methotrexate, minoxidil, tretinoin (ATRA); (59) dermatological corticosteroid anti-inflammatory agents, such as betamethasone and dexamethasone; (60) antifungal topical antiinfectives, such as amphotericin B, clotrimazole, miconazole, and nystatin; (61) antiviral topical anti-infectives, such as acyclovir; (62) topical antineoplastics, such as fluorouracil (5-FU); (63) electrolytic and renal agents, such as lactulose; (64) loop diuretics, such as furosemide; (65) potassium-sparing diuretics, such as triamterene; (66) thiazide diuretics, such as hydrochlorothiazide (HCTZ); (67) uricosuric agents, such as probenecid; (68) enzymes such as RNase and DNase; (69) immunosupressive agents, such as cyclosporine, steroids, methotrexate tacrolimus, sirolimus, rapamycin; (70) antiemetics, such as prochlorperazine; (71) salicylate gastrointestinal anti-inflammatory agents, such as sulfasalazine; (72) gastric acid-pump inhibitor anti-ulcer agents, such as omeprazole; (73) H2-blocker anti-ulcer agents, such as cimetidine, famotidine, nizatidine, and ranitidine; (74) digestants, such as pancrelipase; (75) prokinetic agents, such as erythromycin; (76) opiate agonist intravenous anesthetics such as fentanyl; (77) hematopoietic antianemia agents, such as erythropoietin, filgrastim (G-CSF), and sargramostim (GM-CSF); (78) coagulation agents, such as antihemophilic factors 1-10 (AHF 1-10); (79) anticoagulants, such as warfarin, heparin, and argatroban; (80) growth receptor inhibitors, such as erlotinib and gefetinib; (82) abortifacients, such as methotrexate; (83) antidiabetic agents, such as insulin; (84) oral contraceptives, such as estrogen and progestin; (85) progestin contraceptives, such as levonorgestrel and norgestrel; (86) estrogens such as conjugated estrogens, diethylstilbestrol (DES), estrogen (estradiol, estrone, and estropipate); (87) fertility agents, such as clomiphene, human chorionic gonadatropin (HCG), and menotropins; (88) parathyroid agents such as calcitonin; (89) pituitary hormones, such as desmopressin, goserelin, oxytocin, and vasopressin (ADH); (90) progestins, such as medroxyprogesterone, norethindrone, and progesterone; (91) thyroid hormones, such as levothyroxine; (92) immunobiologic agents, such as interferon beta-1b and interferon gamma-1b; (93) immunoglobulins, such as immune globulin IM, IMIG, IGIM and immune globulin IV, IVIG, IGIV; (94) amide local anesthetics, such as lidocaine; (95) ester local anesthetics, such as benzocaine and procaine; (96) musculoskeletal corticosteroid anti-inflammatory agents, such as beclomethasone, betamethasone, cortisone, dexamethasone, hydrocortisone, and prednisone; (97) musculoskeletal anti-inflammatory immunosuppressives, such as azathioprine, cyclophosphamide, and methotrexate; (98) musculoskeletal nonsteroidal anti-inflammatory drugs (NSAIDs), such as diclofenac, ibuprofen, ketoprofen, ketorlac, and naproxen; (99) skeletal muscle relaxants, such as baclofen, cyclobenzaprine, and diazepam; (100) reverse neuromuscular blocker skeletal muscle relaxants, such as pyridostigmine; (101) neurological agents, such as nimodipine, riluzole, tacrine and ticlopidine; (102) anticonvulsants, such as carbamazepine, gabapentin, lamotrigine, phenytoin, and valproic acid; (103) barbiturate anticonvulsants, such as phenobarbital and primidone; (104) benzodiazepine anticonvulsants, such as clonazepam, diazepam, and lorazepam; (105) anti-parkisonian agents, such as bromocriptine, levodopa, carbidopa, and pergolide; (106) anti-vertigo agents, such as meclizine; (107) opiate agonists, such as codeine, fentanyl, hydromorphone, methadone, and morphine; (108) opiate antagonists, such as naloxone; (109) beta-blocker anti-glaucoma agents, such as timolol; (110) miotic anti-glaucoma agents, such as pilocarpine; (111) ophthalmic aminoglycoside antiinfectives, such as gentamicin, neomycin, and tobramycin; (112) ophthalmic quinolone anti-infectives, such as ciprofloxacin, norfloxacin, and ofloxacin; (113) ophthalmic corticosteroid anti-inflammatory agents, such as dexamethasone and prednisolone; (114) ophthalmic nonsteroidal anti-inflammatory drugs (NSAIDs), such as diclofenac; (115) antipsychotics, such as clozapine, haloperidol, and risperidone; (116) benzodiazepine anxiolytics, sedatives and hypnotics, such as clonazepam, diazepam, lorazepam, oxazepam, and prazepam; (117) psychostimulants, such as methylphenidate and pemoline; (118) antitussives, such as codeine; (119) bronchodilators, such as theophylline; (120) adrenergic agonist bronchodilators, such as albuterol; (121) respiratory corticosteroid anti-inflammatory agents, such as dexamethasone; (122) antidotes, such as flumazenil and naloxone; (123) heavy metal antagonists/chelating agents, such as penicillamine; (124) deterrent substance abuse agents, such as disulfiram, naltrexone, and nicotine; (125) withdrawal substance abuse agents, such as bromocriptine; (126) minerals, such as iron, calcium, and magnesium; (127) vitamin B compounds, such as cyanocobalamin (vitamin B12) and niacin (vitamin B3); (128) vitamin C compounds, such as ascorbic acid; (129) vitamin D compounds, such as calcitriol; (130) vitamin A, vitamin E, and vitamin E compounds; (131) poisons, such as racin; (132) anti-bleeding agents, such as protamine; (133) antihelminth anti-infectives, such as metronidazole; and (134) sclerosants such as talc, alcohol, and doxycyclin.

In addition to the foregoing, the following less common drugs can also be used: chlorhexidine; estradiol cypionate in oil; estradiol valerate in oil; flurbiprofen; flurbiprofen sodium; ivermectin; levodopa; nafarelin; and somatropin. Further, the following drugs can also be used: recombinant beta-glucan; bovine immunoglobulin concentrate; bovine superoxide dismutase; the formulation comprising fluorouracil, epinephrine, and bovine collagen; recombinant hirudin (r-Hir), HIV-1 immunogen; human anti-TAC antibody; recombinant human growth hormone (r-hGH); recombinant human hemoglobin (r-Hb); recombinant human mecasermin (r-IGF-1); recombinant interferon beta-1a; lenograstim (G-CSF); olanzapine; recombinant thyroid stimulating hormone (r-TSH); and topotecan. Further still, the following intravenous products can be used: acyclovir sodium; aldesleukin; atenolol; bleomycin sulfate, human calcitonin; salmon calcitonin; carboplatin; carmustine; dactinomycin, daunorubicin HCl; docetaxel; doxorubicin HCl; epoetin alpha; etoposide (VP-16); fluorouracil (5-FU); ganciclovir sodium; gentamicin sulfate; interferon alpha; leuprolide acetate; meperidine HCl; methadone HCl; methotrexate sodium; paclitaxel; ranitidine HCl; vinblastin sulfate; and zidovudine (AZT).

Further specific examples of useful pharmaceutical agents from the above categories include: (a) anti-neoplastics such as androgen inhibitors, antimetabolites, cytotoxic agents, receptor inhibitors, and immunomodulators; (b) anti-tussives such as dextromethorphan, dextromethorphan hydrobromide, noscapine, carbetapentane citrate, and chlorphedianol hydrochloride; (c) antihistamines such as chlorpheniramine maleate, phenindamine tartrate, pyrilamine maleate, doxylamine succinate, and phenyltoloxamine citrate; (d) decongestants such as phenylephrine hydrochloride, phenylpropanolamine hydrochloride, pseudoephedrine hydrochloride, and ephedrine; (e) various alkaloids such as codeine phosphate, codeine sulfate and morphine; (f) mineral supplements such as potassium chloride, zinc chloride, calcium carbonates, magnesium oxide, and other alkali metal and alkaline earth metal salts; (g) ion exchange resins such as cholestryramine; (h) anti-arrhythmics such as N-acetylprocainamide; (i) antipyretics and analgesics such as acetaminophen, aspirin and ibuprofen; (j) appetite suppressants such as phenyl-propanolamine hydrochloride or caffeine; (k) expectorants such as guaifenesin; (l) antacids such as aluminum hydroxide and magnesium hydroxide; (m) biologicals such as peptides, polypeptides, proteins and amino acids, hormones, interferons or cytokines, and other bioactive peptidic compounds, such as interleukins 1-18 including mutants and analogues, RNase, DNase, luteinizing hormone releasing hormone (LHRH) and analogues, gonadotropin releasing hormone (GnRH), transforming growth factor-β. (TGF-beta), fibroblast growth factor (FGF), tumor necrosis factor-alpha & beta (TNF-alpha & beta), nerve growth factor (NGF), growth hormone releasing factor (GHRF), epidermal growth factor (EGF), fibroblast growth factor homologous factor (FGFHF), hepatocyte growth factor (HGF), insulin growth factor (IGF), invasion inhibiting factor-2 (IIF-2), bone morphogenetic proteins 1-7 (BMP 1-7), somatostatin, thymosin-alpha-1, gamma-globulin, superoxide dismutase (SOD), complement factors, hGH, tPA, calcitonin, ANF, EPO and insulin; (n) anti-infective agents such as antifungals, anti-virals, antihelminths, antiseptics and antibiotics; and (m) oxygen, hemoglobin, nitric or sliver oxide.

Non-limiting examples of broad categories of useful pharmaceutical agents include the following therapeutic categories: anabolic agents, anesthetic agents, antacids, anti-asthmatic agents, anticholesterolemic and anti-lipid agents, anti-coagulants, anti-convulsants, anti-diarrheals, antiemetics, anti-infective agents, anti-inflammatory agents, anti-manic agents, anti-nauseants, antineoplastic agents, anti-obesity agents, anti-pyretic and analgesic agents, anti-spasmodic agents, anti-thrombotic agents, anti-uricemic agents, anti-anginal agents, antihistamines, anti-tussives, appetite suppressants, biologicals, cerebral dilators, coronary dilators, decongestants, diuretics, diagnostic agents, erythropoietic agents, expectorants, gastrointestinal sedatives, hyperglycemic agents, hypnotics, hypoglycemic agents, ion exchange resins, laxatives, mineral supplements, mucolytic agents, neuromuscular drugs, peripheral vasodilators, psychotropics, sedatives, stimulants, thyroid and anti-thyroid agents, uterine relaxants, vitamins, and prodrugs.

Some non-limiting examples of specific drugs that can be used include: asparaginase, bleomycin, busulfan, capecitabine, carboplatin, carmustine, chlorambucil, cisplatin, cyclophosphamide, cytarabine, dacarbizine, dactinomycin, daunorubicin, dexrazoxane, docetaxel, doxorubicin, etoposide, floxuridine, fludarabine, fluoruracil, gemcitabine, hydroxyurea, idarubicin, ifosfamide, irinotecan, lomustine, mechlorethamine, melphalan, mercaptopurine, methotrexate, mitomycin, mitotane, mitoxantrone, paclitaxel, pentostatin, plicamycin, premextred procarbazine, rituximabe, streptozocin, teniposid, thioguanine, thiotepa, vinplastine, vinchristine, and vinorelbine. In some embodiments, the drugs for lung cancer treatment is paclitaxel, pemetrexed, 10-hydrocamptothecin, irinotecan, erlotinibil/gefetinib or derivates of these molecules.

Examples of anticancer, antineoplastic agents are camptothecins. These drugs are antineoplastic by virtue of their ability to inhibit topoisomerase I. Camptothecin is a plant alkaloid isolated from trees indigenous to China and analogs thereof such as 9-aminocamptothecin, 9-nitrocamptothecin, 10-hydroxycamptothecin, 10,11-methylenedioxycamptothecin, 9-nitro-10,11-methylenehydroxycamptothecin, 9-chloro-10,11-methylenehydroxycamptothecin, 9-amino-10,11-methylenehydroxycamptothecin, 7-ethyl-10-hydroxycamptothecin (SN-38), topotecan, DX-8951, Lurtotecan (GII147221C), and other analogs (collectively referred to herein as camptothecin drugs) are presently under study worldwide in research laboratories for treatment of colon, breast, and other cancers.

Additionally, the pharmaceutical agent can be a radiosensitizer, such as metoclopramide, sensamide or neusensamide (manufactured by Oxigene); profiromycin (made by Vion); RSR13 (made by Allos); THYMITAQ® (made by Agouron), etanidazole or lobenguane (manufactured by Nycomed); gadolinium texaphrin (made by Pharmacyclics); BuDR/Broxine (made by NeoPharm); IPdR (made by Sparta); CR2412 (made by Cell Therapeutic); LlX (made by Terrapin); agents that minimize hypoxia, and the like.

The agent can be selected from a biologically active substance. The biologically active substance can be selected from the group consisting of peptides, poly-peptides, proteins, amino acids, polysaccharides, growth factors, hormones, anti-angiogenesis factors, interferons or cytokines, elements, and pro-drugs. In some embodiments, the biologically active substance is a therapeutic drug or pro-drug; in other embodiments, a drug is selected from the group consisting of chemotherapeutic agents and other antineoplastics such as paclitaxel, antibiotics, anti-virals, antifungals, anesthetics, antihelminths, anti-inflammatories, and anticoagulants. In certain useful embodiments, the therapeutic drug or pro-drug is selected from the group consisting of chemotherapeutic agents and other antineoplastics such as paclitaxel, carboplatin and cisplatin; nitrosourea alkylating antineoplastic agents, such as carmustine (BCNU); fluorouracil (5-FU) and gemcitabine; hormonal antineoplastics, such as goserelin, leuprolide, and tamoxifen; receptor inhibitors such as erlotinib, gefetinib, sutent or anti-ckit inhibitors, such as GLEEVEC®; natural antineoplastics, such as aldesleukin, interleukin-2, docetaxel, etoposide (VP-16), interferon alpha, paclitaxel, and tretinoin (ATRA).

In another embodiment, the biologically active substance is a nucleic acid molecule. The nucleic acid molecule's sequence can be selected from among any DNA or RNA sequence. In certain embodiments, the biologically active substance is a DNA molecule that encodes a genetic marker selected from among luciferase gene, β-galactosidase gene, resistance, neomycin resistance, and chloramphenicol acetyl transferase. In certain embodiments, the biologically active substance is a DNA molecule that encodes a gene product (e.g., lectin, a mannose receptor, a sialoadhesin, or a retroviral transactivating factor). In certain embodiments, the biologically active substance is a DNA molecule that encodes an RNA selected from the group consisting of a sense RNA, an antisense RNA, siRNA and a ribozyme.

Biologically active agents amenable for use with the new polymers described herein include, without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment. Useful active agents amenable for use in the new compositions include growth factors, such as transforming growth factors (TGFs), fibroblast growth factors (FGFs), platelet derived growth factors (PDGFs), epidermal growth factors (EGFs), connective tissue activated peptides (CTAPs), osteogenic factors, and biologically active analogs, fragments, and derivatives of such growth factors. Members of the transforming growth factor (TGF) supergene family, which are multifunctional regulatory proteins, are preferred. Members of the TGF supergene family include the beta-transforming growth factors (for example, TGF-b1, TGF-b2, and TGF-b3); bone morphogenetic proteins (for example, BMP-1, BMP-2, BMP-3, BMP-4, BMP-5, BMP-6, BMP-7, BMP-8, and BMP-9); heparin-binding growth factors (for example, fibroblast growth factor (FGF), epidermal growth factor (EGF), platelet-derived growth factor (PDGF), and insulin-like growth factor (IGF)); inhibins (for example, Inhibin A, Inhibin B); growth differentiating factors (for example, GDF-1); and activins (for example, Activin A, Activin B, and Activin AB).

In some embodiments, the bioactive agent(s) is/are independently selected from the group consisting of an antibiotic, an antimitotic, an anti-inflammatory agent, a growth factor, a targeting compound, a cytokine, an immunotoxin, an anti-tumor antibody, an anti-angiogenic agent, an anti-edema agent, a radiosensitizer, and a chemotherapeutic. In some embodiments, at least one of the bioactive agents is camptothecin. In some embodiments, at least one of the bioactive agents is 10-hydroxycamptothecin. In some embodiments, at least one of the bioactive agents is paclitaxel.

In some embodiments, at least one of the one or more independently selected bioactive agents is a platinum containing molecule. In some embodiments, the platinum containing molecule is selected from the group consisting of cisplatin and carboplatinum.

In some embodiments, at least one of the one or more independently selected bioactive agents is a chemotherapeutic agent. In some embodiments, the chemotherapeutic agent is present in at least one of the one or more polymer coatings at a loading of from about one-tenth to about 80 percent by weight. In some embodiments, the chemotherapeutic agent is a drug useful for treating breast, ovarian, or non-small cell lung cancer.

In some embodiments, the chemotherapeutic agent is released from the composition with linear or first order kinetics. In some embodiments, the chemotherapeutic agent is released from the composition over a time frame effective to inhibit tumor growth or prevent metastasis when the composite is affixed to the tissue surface at the site of disease. In some embodiments, the chemotherapeutic agent is released from the composition over a time frame effective to prevent tumor recurrence when the composite is affixed to tumor resection margins following surgery.

In some embodiments, the bioactive agent is paclitaxel.

In some embodiments, at least one of the one or more independently selected bioactive agents is released from the composite over a time frame of at least about 7 days, when affixed to a tissue surface. In some embodiments, the time frame is at least about 30 days. In some embodiments, the time frame is at least about 60 days. In some embodiments, at least one bioactive agent is present in the composition at a loading of from about one-tenth to about 80 percent by weight.

The bioactive agent can localize differently in the 3-dimensional compositions described herein based on the selected bioactive agent, the concentration of bioactive agent, the polymer and hydrophobic dopant, and the fabrication method. For example, when electrospinning 1 wt % SN-38 within PCL with no hydrophobic dopant, and PCL with 10% hydrophobic dopant, drug segregates mostly to the core of fibers. When decreasing the SN-38 to 0.1% or 0.01% using the same polymer compositions, the drug is mostly observed at the surface.

### 3-Dimensional Compositions

The 3-dimensional compositions described herein can comprise any shape including, but not limited to, pellets, droplets, beads, fibers (e.g., nanofibers or microfibers), fibrous mats, or more complex structures (e.g., tubes, implants etc.). In one embodiment, the 3-dimensional compositions comprise substantially homogeneous properties throughout the bulk of the composition (e.g., a consistent contact angle). A 3-dimensional composition as described herein is distinguished from a 2D coating by their interaction with their environment. For example, if a 2D coating is submerged in water, only the actual material surface (a 2D surface in X and Y) ever contacts the water, where the remainder of the material (the bulk material in Z) remains unexposed. This is in contrast to a 3D material/coating, where rather than being a singular discrete surface, instead there are many surfaces through the thickness of the material, and the water can potentially interact with the entirety of the material/coating.

For the purposes of this application, a 2D coating is defined as having a surface of 1 micron or less, while a 3D coating/material is defined as having a surface or depth greater than 1 micron. One of skill in the art will appreciate that a 2D surface comprising a depth or thickness of one micron or less will not have enough agent incorporated bioactive agent to result in release of a bioactive agent in a therapeutically relevant amount and therefore does not permit a desired therapeutic response. In contrast, a 3-dimensional material/coating comprises both depth and volume such that enough bioactive agent can be loaded to achieve a desired response upon administration to a subject.

In some embodiments, the compositions described herein comprise more than one polymer in combination with a hydrophobic doping agent, for example, the composition can comprise at least 2, at least 3, at least 4, at least 5, at least 6, at least 7, at least 8, at least 9, at least 10, or more polymers in combination with a hydrophobic doping agent.

In some embodiments, the 3-dimensional composition is manufactured using e.g., electrospraying, electrospinning, ultrasonic spraying, dip-coating, vapor deposition, spin-coating, knife-coating, melt-coating, or injection molding.

In one embodiment, the compositions described herein are porous. For example, the composition can have a porosity of greater than 5% by volume, greater than 10% by volume, greater than 15% by volume, greater than 20% by volume, greater than 25% by volume or more.

In one embodiment, the compositions described herein comprise entrapped air. In alternative embodiments, the compositions described herein comprise an entrapped gas such as, argon, helium, nitrogen, among others.

The compositions described herein comprise entrapped gas (e.g., air) and permit release of the bioactive agent upon controlled gas (e.g., air) removal from the composition. In some embodiments, the e.g., air is maintained at the surface of the composition and/or within the bulk of the composition for at least 1 hour, at least 2 hours, at least 3 hours, at least 6 hours, at least 12 hours, at least 24 hours, at least 36 hours, at least 48 hours, at least 7 days, at least 2 weeks, at least 15 days, at least 20 days, at least three weeks, at least 25 days, at least 4 weeks, at least 30 days, at least 35 days (e.g., 5 weeks), at least 40 days, at least 6 weeks, at least 45 days, at least 7 weeks, at least 50 days, at least 55 days, at least 8 weeks, at least 60 days, at least 9 weeks, at least 65 days, at least 70 days, at least 75 days, at least 11 weeks, at least 80 days, at least 85 days, at least 90 days, at least 95 days, at least 100 days or more in an aqueous solution or other liquid.

In some embodiments, the compositions release the bioactive agent 20% faster over a given period of time (e.g., 24 hours) when the air content at the surface and/or within the composition is displaced upon exposure to an environmental trigger such as ultrasound, strain, or injection of a surfactant/solvent (e.g., ethanol).

In one embodiment, the 3-dimensional composition as described herein comprises a fiber, for example, a nanofiber or a microfiber. Fibers can be produced using any method known in the art such as, melt spinning, extrusion, drawing, wet spinning, electrospray, or electrospinning. In one embodiment, the fibers are produced using electrospinning. Electrospinning can be performed by any means known in the art (see, for example, U.S. Pat. No. 6,110,590).

In one embodiment, the diameter of the fiber is between about 10 nm and about 50 nm. In another embodiment, the diameter of the fiber is between about 10 nm and 500 nm. In another embodiment, the diameter of the fiber is between about 100 nm and 300 nm. In another embodiment, the diameter of the fiber is between about 100 nm and 500 nm. In another embodiment, the diameter of the fiber is between about 50 nm and 400 nm. In another embodiment, the diameter of the fiber is between about 200 nm and 500 nm. In another embodiment, the diameter of the fiber is between about 300 nm and 600 nm. In another embodiment, the diameter of the fiber is between about 400 nm and 700 nm. In another embodiment, the diameter of the fiber is between about 500 nm and 800 nm. In another embodiment, the diameter of the fiber is between about 500 nm and 1000 nm. In another embodiment, the diameter of the fiber is between about 1000 nm and 1500 nm. In another embodiment, the diameter of the fiber is between about 1500 nm and 3000 nm. In another embodiment, the diameter of the fiber is between about 2000 nm and 5000 nm. In another embodiment, the diameter of the fiber is between about 3000 nm and 4000 nm.

In one embodiment, the 3-dimensional composition comprises a bead or droplet. In such embodiments, the average diameter of a bead is between 500 nm and 10000 nm, or alternatively, between 500 nm and 1000 nm, between 1000 nm and 1500 nm, between 1500 nm and 2000 nm, between 2000 nm and 2500 nm, between 2500 nm and 3000 nm, between 3000 nm and 3500 nm, between 3500 nm and 4000 nm, between 4000 nm and 4500 nm, between 4500 nm and 5000 nm, between 5000 nm and 5500 nm, between 5500 nm and 6000 nm, between 6000 nm and 6500 nm, between 6500 nm and 7000 nm, between 7000 nm and 7500 nm, between 7500 nm and 8000 nm, between 8000 nm and 8500 nm, between 8500 nm and 9000 nm, between 9000 nm and 9500 nm, between 9500 nm and 10000 nm.

In one embodiment, said aforementioned surface is a superhydrophobic fiber mat comprising a plurality of the aforementioned fibers. In one embodiment, said superhydrophobic fiber mat is electrospun. In another embodiment, said superhydrophobic fiber mat exhibits wettability properties. In another embodiment, the fibers within the mat are uniform. In another embodiment, the mat is composed solely of fibers randomly oriented in a plane.

In some embodiments of the compositions described herein, the 3-dimensional composition comprises at least one pore having a pore size of e.g., between 0.01 microns to 100 microns, between 0.1 microns to 100 microns, between 0.1 microns to 50 microns, between 0.1 microns to 10 microns, between 0.1 microns to 5 microns, between 0.1 microns to 2 microns, between 0.2 microns to 1.5 microns. In another embodiment, the pore size can be non-uniform. In another embodiment, the pore size can be uniform.

In some embodiments, the composition comprises multiple layers, e.g., at least 2 layers, at least 3 layers, at least 4 layers, at least 5 layers or more.

In one embodiment, the compositions described herein are not oleophobic.

### Formulation and Administration

In one aspect, the methods described herein provide a method for delivering an agent to a subject in need thereof. In one embodiment, the subject is a mammal. In another embodiment, the mammal is a human, although the approach is effective with respect to all mammals. The method comprises administering to the subject an effective amount of a pharmaceutical composition comprising an agent encapsulated within a protein cage, in a pharmaceutically acceptable carrier.

The dosage range for an agent depends upon the potency, and includes amounts large enough to produce the desired effect, e.g., a reduction in a symptom or marker of a disease. The dosage should not be so large as to cause unacceptable adverse side effects. Generally, the dosage of an agent will vary with the type of agent (e.g., an antibody or fragment, small molecule, siRNA, etc.), and with the age, condition, and sex of the patient. The dosage can be determined by one of skill in the art and can also be adjusted by the individual physician in the event of any complication. Typically, the dosage ranges for a free drug (i.e., not in a polymer drug delivery device) are from 0.001 mg/kg body weight to 5 g/kg body weight. In some embodiments, the dosage range for a free drug is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range for a free drug is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 μg/kg body weight to 30 μg/kg body weight. Alternatively, the dose range will be titrated to maintain serum levels between 5 μg/mL and 30 μg/mL. The dose of a bioactive agent delivered by the compositions described herein can be tailored to produce a similar free drug concentration (e.g., a therapeutically effective concentration) in e.g., blood as is achieved using a standard method of administration of the free drug.

Given the ability of a bioactive agent in a composition as described herein to provide sustained release of a free bioactive agent over time, it is also contemplated that the dose of the agent present in the polymeric composition is higher than the amount of free agent administered alone. This aspect is especially important for reducing dose-limiting toxicities of a free agent by permitting a slow, sustained release of a therapeutic amount of an agent from a polymeric composition. Thus, the amount of a bioactive agent administered using the compositions described herein is at least 5% higher than the dose necessary for a free drug to produce an equivalent effect (e.g., 50% reduction in a symptom or marker of disease); preferably the amount of an agent administered with the polymeric composition is at least 10% higher, at least 20% higher, at least 30% higher, at least 40% higher, at least 50% higher at least 60% higher, at least 70% higher, at least 80% higher, at least 90% higher, at least 95% higher, at least 1-fold higher, at least 2-fold higher, at least 5-fold higher, at least 50-fold higher, at least 100-fold higher, at least 1000-fold higher or more than the amount of free agent administered to achieve an equivalent bioactive effect.

Administration of the doses recited above can be repeated for a limited period of time. In some embodiments, the doses are given once a day, or multiple times a day, for example but not limited to three times a day. In one embodiment, the doses recited above are administered daily for several weeks or months. The duration of treatment depends upon the subject's clinical progress and responsiveness to therapy. Continuous, relatively low maintenance doses are contemplated after an initial higher therapeutic dose. It will be clear to one of skill in the art that the slow-release properties of the polymeric compositions described herein permit the compositions to be administered less frequently than that of the free drug. For example, the polymeric compositions described herein can be administered every 36 h, every 48 h, every 3 days, every 4 days, every 5 days, every 6 days, every week, every two weeks, every three weeks, every four weeks, ever six weeks, or longer. In some embodiments, the compositions described herein are administered only once, for example, the composition is implanted near a tumor or other site near the tissue one wishes to target, or otherwise administered as a bolus composition. In one embodiment, a composition releases the bioactive agent substantially continuously at a therapeutic dose for at least 7 days, at least 10 days, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks or longer.

Agents useful in the methods and compositions described herein can be administered topically, intravenously (by bolus or continuous infusion), orally, by inhalation, intraperitoneally, intramuscularly, subcutaneously, intracavity, and can be delivered by peristaltic means, if desired, or by other means known by those skilled in the art. The agent can be administered systemically, or alternatively, can be administered directly to a desired site, e.g., a tumor e.g., by intratumor injection, implantation near or on the tumor, or by injection into the tumor's primary blood supply.

Therapeutic compositions containing at least one agent can be conventionally administered in a unit dose. The term “unit dose” when used in reference to a therapeutic composition refers to physically discrete units suitable as unitary dosage for the subject, each unit containing a predetermined quantity of active material calculated to produce the desired therapeutic effect in association with the required physiologically acceptable diluent, i.e., carrier, or vehicle.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired.

Precise amounts of active ingredient required to be administered depend on the judgment of the practitioner and are particular to each individual. However, suitable dosage ranges for systemic application are disclosed herein and depend on the route of administration. Suitable regimes for administration are also variable, but are typified by an initial administration followed by repeated doses at one or more intervals by a subsequent injection or other administration.

In some embodiments, the drug-eluting composition is administered on the surface of cancerous tissue or the site remaining after surgical resection and releases one or more anticancer agents in a gradual and prolonged manner to reduce or kill tumors and/or prevent recurrence or metastasis in tissues including but not limited to lung, colon, ovary, pancreas, mesothelium, connective tissue, stomach, liver, and kidney. As such, these drug-eluting compositions are of use for treating sarcomas, mesothelioma, lung cancer, breast cancer, colon cancer, or ovarian cancer, among others. In some embodiments, the composition is administered to the resection margins after local surgery following the removal of a tumor to destroy residual remaining disease and prevent recurrence. The composition can be loaded with one or more prohealing drugs such as anti-inflammatories in addition to anticancer agents to ensure adequate healing of noncancerous tissue. In some embodiments, the composition is implanted e.g., stapled directly over the surface of diseased or treated tissue. The implants can also be combined with other therapeutic modalities, including radiotherapy, other chemotherapeutic agents administered systemically or locally, immunotherapy, or radiofrequency ablation. In some embodiments, the implant is administered to the site of disease utilizing methods currently used during standard surgical resection procedures, for example by simultaneously administering the composite using the surgical stapler during the removal of the primary tumor. By the appropriate selection of polymer, doping agent, and bioactive agent, a flexible implant capable of controlled release of a therapeutic agent to the surface of a tissue can be constructed.

In some embodiments, a chemotherapeutic agent is released at the site of disease for at least 7 days, at least 10 days, at least two weeks, at least 3 weeks, at least 1 month, at least 2 months, at least 3 months, at least 6 months or more.

In some embodiments, the implant is surgically stapled in direct contact with the tissue surface at the site of disease. In some embodiments, the implant is affixed in direct contact with the tissue surface at the site of disease using an adhesive or glue. The methods of administration can be used to administer any of the embodiments of the compositions described herein, or combination thereof.

It is further appreciated that certain features of the invention, which are, for clarity, described in the context of separate embodiments, can also be provided in combination in a single embodiment.

### Efficacy Measurement

The efficacy of a given treatment for a disease can be determined by the skilled clinician. However, a treatment is considered “effective treatment,” as the term is used herein, if any one or all of the signs or symptoms of the disease are altered in a beneficial manner, other clinically accepted symptoms or markers of disease are improved, or ameliorated, e.g., by at least 10% following treatment with a polymeric composition as described herein. Efficacy can also be measured by failure of an individual to worsen as assessed by hospitalization or need for medical interventions (i.e., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the development of the disease; or (2) relieving the disease, e.g., causing regression of symptoms; and (3) preventing or reducing the likelihood of the development of a disease.

An effective amount for the treatment of a disease means that amount which, when administered to a mammal in need thereof, is sufficient to result in effective treatment as that term is defined herein, for that disease. Efficacy of an agent can be determined by assessing physical indicators of, for example cancer, such as e.g., tumor size, tumor growth rate, etc.

### Other Embodiments

Provided herein are compositions from which one or more therapeutic agents can be released in a controlled manner, as well as methods and uses of said compositions such as for the treatment and/or prevention of cancer. Many of the compositions described herein can be used for the controlled, localized, and sustained delivery of various bioactive agents (i.e. drugs) for treatment of a variety of diseases and/or conditions including treatment for malignancy, pain, infection, inflammation, resistance to surgical adhesions, healing of ulcers, cosmesis, immunization and autoimmune dysfunction. In one aspect, the compositions and methods described herein pertain to compositions comprising: a) a biodegradable polymeric nanofiber or microfiber; and b) a hydrophobic doping agent comprising a polymer that is different from the biodegradable polymeric nanofiber or microfiber (i.e. collectively (a) and (b) represent the polymeric carrier). In some embodiments, the composition can also comprise: (a) a bioactive agent (such as an anti-cancer agent). Some non-limiting examples of anti-cancer agents include asparaginase, bleomycin, busulfan, capecitabine, carboplatin, carmustine, chlorambucil, cisplatin, cyclophosphamide, cytarabine, dacarbizine, dactinomycin, daunorubicin, dexrazoxane, docetaxel, doxorubicin, etoposide, floxuridine, fludarabine, fluoruracil, gemcitabine, hydroxyurea, idarubicin, ifosfamide, irinotecan, lomustine, mechlorethamine, melphalan, mercaptopurine, methotrexate, mitomycin, mitotane, mitoxantrone, paclitaxel, pentostatin, plicamycin, premextred procarbazine, rituximabe, streptozocin, teniposid, thioguanine, thiotepa, vinplastine, vinchristine, and vinorelbine.

A wide variety of polymers can be utilized in the composition, including, for example, oligomers and polymers consisting of poly(caprolactone), polylactide, polyglycolide, poly(lactide-co-glycolide), poly(dioxanone), poly(trimethylene carbonate), poly(ethylene glycol), pluronics (poly(ethylene glycol-co-propylene glycol), and poly(glycerol monostearate-co-caprolactone) or copolymers or blends thereof.

A wide variety of hydrophobic doping agents can be utilized in the composition, including, for example, polymers, oligomers, or small molecules of greater hydrophobicity than the primary composition material, in order to significantly prolong and/or graduate release of embedded therapeutic agents as compared to the non-doped composition. Contact angle measurement is a primary method for characterizing the hydrophobicity of a material; contact angles>90° are generally considered hydrophobic materials. The composite contact angle of two blended polymers is customarily predicted to fall within the range of the two polymers' respective contact angles, skewed in relative proportion towards the polymer dominant in the blend. In one embodiment, the introduction of a small mass percentage<20 weight % of a hydrophobic polymer with a contact angle>100° into a blend with another more hydrophilic polymer with a contact angle<90°, results in a contact angle that is disproportionately skewed towards the higher contact angle as a fraction of mass percent of the two components.

Within one embodiment, hydrophobic doping agents can be utilized to prolong or alter the degradation rate of the device. In one embodiment, the hydrophobic doping agent has a contact angle that is at least 10° greater than the primary polymer(s) in the blend. In another embodiment, the incorporation of less than 20% by weight of hydrophobic doping agents increases or decreases the average drug release and/or degradation kinetics of the polymer matrix by greater than 50%. In another embodiment, the incorporation of less than 10% by weight of hydrophobic doping agents increases or decreases the average drug release and/or degradation kinetics of the polymer matrix by greater than 20%.

It is also contemplated that the doping agent can be a hydrophilic polymer including, for example, poly(ethylene glycol) or poly(ethylene glycol-co-propylene glycol)/pluronics. In another embodiment, the incorporation of less than 20% by weight of hydrophilic doping agents increases or decreases the average drug release and/or degradation kinetics of the polymer matrix by greater than 50%. In one embodiment, a hydrophilic doping agent has a contact angle that is at least 60° less than the primary polymer(s) in the blend.

In another embodiment, both a hydrophobic and a hydrophilic doping agent are incorporated into a blend, with both individually comprising less than 20% by weight of the polymer blend. In a preferred embodiment, the incorporation of less than 20% by weight of hydrophobic doping agents increases or decreases the average drug release and/or degradation kinetics of the polymer matrix by greater than 50%. In a further embodiment, inclusion of the hydrophilic polymer facilitates the loading of hydrophilic agents into the hydrophobic polymer blend for slow and controlled drug release by increasing the partition coefficient of the agent into the hydrophobic polymer blend.

With one embodiment of the compositions and methods described herein, the composition is processed into a non-woven mesh with an average thickness between 0.05 to 1000 μm. In another embodiment, the device comprises multiple layers of mesh, in which one or more layers contain a therapeutic agent. In another embodiment, drug release is uni-directional. In another embodiment, the mesh is flexible and not rigid. In another embodiment, the polymer composition is biodegradable and/or biocompatible.

In one embodiment of the compositions and methods described herein, the composition is processed into a non-woven or woven mesh such that the surface exhibits roughness to increase the hydrophobicity as measured by an increase of contact angle of 10° or more over composition that has been cast from solvent or melt processed. In another embodiment, the device is comprised of multiple layers of mesh, in which one or more layers comprise a therapeutic agent. In another embodiment, the mesh is flexible and not rigid. In another embodiment, the polymer composition is biodegradable and biocompatible.

In another embodiment, methods are provided for treating surgical resection margins, comprising anti-cancer compositions as those described following the surgical excision of tumor, such that the local recurrence of cancer is inhibited. In another embodiment, methods are provided for treating tissues containing or adjacent to lymphatic tissues, such that the migration of tumor cells, or metastasis, is inhibited.

In another embodiment, compositions and methods are provided for preparation and use of the polymer-based compositions as surgical meshes and/or scaffolds with or without seeding of cells for the repair of tissues, for the closure of wounds sites, for the closure of surgically induced wounds/incisions, for the filling of a tissue void space, and for the augmentation of tissue.

In another embodiment, compositions and methods are provided for preparation and use of the polymer-based compositions as compliant, partially compliant, or non-compliant surgical meshes and/or scaffolds with or without drug and with or without radioopaque/radioabsorptive compositions , or radionuclides.

In one embodiment, compositions and methods are provided for use in veterinary applications.

In another embodiment, compositions and methods are provided for preparation and use of the polymer-based compositions as filters for separation of hydrophobic and hydrophilic components of a complex mixture, water purification, as a material used as an antifouling agent, as materials for clothing, as a material for air filtration, as materials for addition to plastics to increase hydrophobicity and strength, or compliance, as materials for high-performance sails, and as an office or home construction material.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

### EXAMPLES

### Example 1

### Formation of Poly(Caprolactone) Non-Woven Meshes with and without a Hydrophobic Doping Agent

Non-woven polymer meshes and blends were prepared using an electrospinning apparatus. Solutions of polycaprolactone were prepared (20 w/v %) in a 5:1 chloroform/methanol mixture with or without the inclusion of 1-20 w/w % poly(glycerol monostearate-co-caprolactone). Each solution was loaded into a glass syringe and placed into a syringe pump set at a flow rate of 25 mL/hr. A 15-18 kV high voltage lead was applied at the base of the syringe needle. A grounded rotating collector was covered in aluminum foil and placed 20-30 cm away from the needle. Following 30-60 minutes of electrospinning, the resulting non-woven polymer meshes were peeled off the aluminum foil backing for future use. Meshes created in this manner have average fiber diameters between 1-10 μm. For poly(glycerol monostearate-co-caprolactone), the monomer ratio in the final polymer was about 80 mol % caprolactone and the molecular weight was about 10,000 Da. The molecular weight for the poly(caprolactone) was between 70,000-90,000 Da.

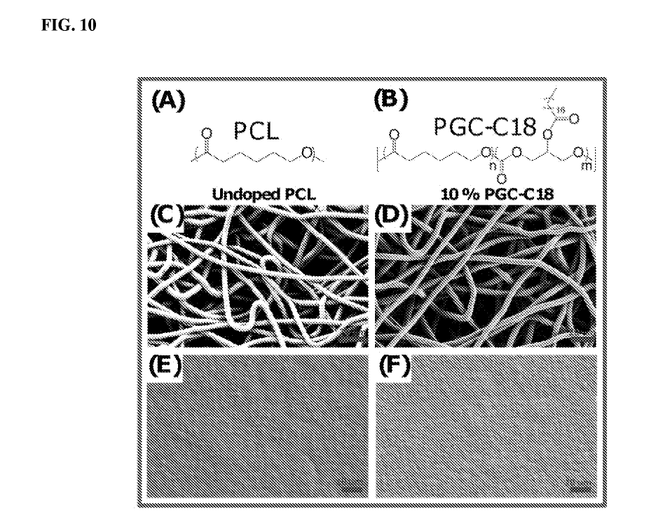

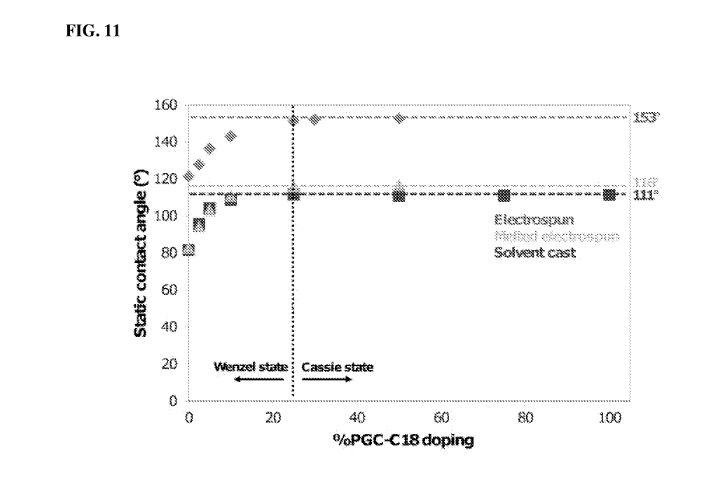

The resulting meshes are 300 μm thick, with an average fiber size of ≈7 μm (FIGS. 10C-D). The wettability of the meshes was assessed using static contact angle measurements, where electrospun PCL meshes doped with PGC-C18 asymptotically approach 153° with 50 wt % doping (FIG. 11). Melted electrospun meshes were prepared by treating meshes at 80° C. for 1 minute followed by quenching to collapse the porous structure on itself (FIGS. 10E-F). This procedure was done quickly to prevent phase separation of PCL and PGC-C18, which was confirmed by differential scanning calorimetry (DSC) and consistent with their similar structures. Electrospun meshes and melted electrospun meshes for PCL and 10% doped PGC-C18 PCL were compared using SEM and showed that the melted meshes have a comparably smooth surface.

The surface roughness of single electrospun fibers was quantified for PCL and PCL doped with 10% PGC-C18 using AFM. Electrospun fibers showed a finite surface roughness (RMS≈50 nm) with consistent RMS values between fibers with different PGC-C18 doping concentrations. This finite roughness indicates that both intrafiber and interfiber roughness may contribute to high apparent contact angles. The melted electrospun meshes afforded a lower maximum contact angle of 116° with 50 wt % doping of PGC-C18. Solvent cast films of the polymers possessed contact angles similar to the melted electrospun meshes) (Θₘₐₓ=111°). Surface area measurement using Kr BET on the electrospun and melted electrospun meshes showed that electrospun meshes possess at least 30× more surface area than the melted counterparts. Electrospun mesh surfaces with <25% PGC-C18 doping could be pushed into the stable Wenzel regime by dropping the water droplet used in contact angle measurements from 2 feet. Electrospun meshes with >25% PGC-C18 doping could not be pushed into the Wenzel regime in this way, indicating that 25% doping is an approximate boundary condition for the Wenzel-to-Cassie state transition.

### Example 2

### Tunability of Polymer Wet-Ability Using a Hydrophobic Doping Agent

Solvent-cast poly(caprolactone) films were prepared containing 0-75 wt % poly(glycerol monostearate-co-caprolactone). The polymers were co-dissolved in dichloromethane (10 w/v %) and films were cast onto glass substrates. Contact angle measurements were obtained as a measure of hydrophobicity/wet-ability of the polymer. The contact angle ranged from ˜83° for films composed solely of poly(caprolactone), and increased up to a maximum of 111° when blended with at least 10% poly(glycerol monostearate-co-caprolactone).

### Example 3

### Release of Camptothecins from Polymer Meshes Containing Various Concentrations of a Hydrophobic Doping Agent

Drug-loaded microfiber meshes containing the camptothecin molecules CPT-11 and SN38 were prepared by the electrospinning procedure outlined in Example 1 using blends of poly(caprolactone) and poly(glycerol monostearate-co-caprolactone) (2.5 and 10 wt %). In vitro drug release was performed in PBS at 37° C. Increasing the weight percent of poly(glycerol monostearate-co-caprolactone) in the meshes led to a decrease of “burst” release kinetics compared with meshes containing a lower weight percent or no weight percent. At seven days following the initiation of the release study using SN38-loaded meshes, poly(caprolactone) released about 37% of its initial drug load, the 2.5 w/w % blend released 26%, and the 10 w/w % blend released 11%. At fourteen days, poly(caprolactone) released about 66% of its initial drug load, the 2.5% blend released 41%, and the 10% blend released 19%. Significant drug release (>1%/week) concluded at 14, 35, and 70 days for the three formulations, respectively. Poly(caprolactone) meshes loaded with CPT-11 released 60% of their initial drug loading over 24 hours and failed to release significant drug (>1%/week) thereafter. Conversely, the 10 w/w % blend released about 5% of initial drug over the first 24 hours of release, followed by gradual drug release over the next 6 weeks, and concluding significant release at 42 days.

### Example 4

### Formation of Poly(Lactide-Co-Glycolide) Non-Woven Meshes with and without a Hydrophobic Doping Agent

Non-woven poly(lactide-co-glycolide) meshes and blends were prepared using an electrospinning apparatus similarly to those of Example 1. Solutions of poly(lactide-co-glycolide) were prepared (30 w/v %) in a 1:1 dichloromethane/DMF mixture with or without the inclusion of 1-20 w/w % poly(glycerol monostearate-co-caprolactone). Each solution was loaded into a glass syringe and placed into a syringe pump set at a flow rate of 5 mL/hr. A 10-25 kV high voltage lead was applied at the base of the syringe needle. A grounded rotating collector was covered in aluminum foil and placed 20-30 cm away from the needle. Following 30-60 minutes of electrospinning, the resulting non-woven polymer meshes were peeled off the aluminum foil backing for future use. Meshes created in this manner have average fiber diameters between 0.2-10 μm. For poly(glycerol monostearate-co-caprolactone), the monomer ratio in the final polymer was about 80 mol % caprolactone and the molecular weight was about 10,000 Da. The molecular weight for the poly(lactide-co-glycolide) was between 50,000-200,000 Da. Poly(lactide-co-glycolide) copolymers were selected with ratios of lactide to glycolide varying from 50:50 to 85:15 mol % lactide.

### Example 5

### Formation of Poly(Lactide-Co-Caprolactone) Non-Woven Meshes with and without a Hydrophobic Doping Agent

Poly(lactide-co-caprolactone) meshes were prepared similarly to the poly(lactide-co-glycolide) meshes in Example 4. Solutions of poly(lactide-co-caprolactone) were prepared (30 w/v %) in a 1:1 dichloromethane/DMF mixture with or without the inclusion of 1-40 w/w % poly(glycerol monostearate-co-caprolactone). Each solution was loaded into a glass syringe and placed into a syringe pump set at a flow rate of 5 mL/hr. A 10-25 kV high voltage lead was applied at the base of the syringe needle. A grounded rotating collector was covered in aluminum foil and placed 20-30 cm away from the needle. Following 30-60 minutes of electrospinning, the resulting non-woven polymer meshes were peeled off the aluminum foil backing for future use. Meshes created in this manner have average fiber diameters between 0.2-10 μm. The molecular weights for the poly(lactide-co-glycolide) were between 25,000-200,000 Da. Poly(lactide-co-caprolactone) copolymers were selected with ratios of lactide to caprolactone varying from 90:10 to 50:50 mol % lactide.

### Examples 6A-6D

### Drug Release from Poly(Caprolactone) Non-Woven Meshes with and without a Hydrophobic Doping Agent

### Example 6A

### SN-38 Release from Poly(Caprolactone) Non-Woven Meshes with and without a Hydrophobic Doping Agent

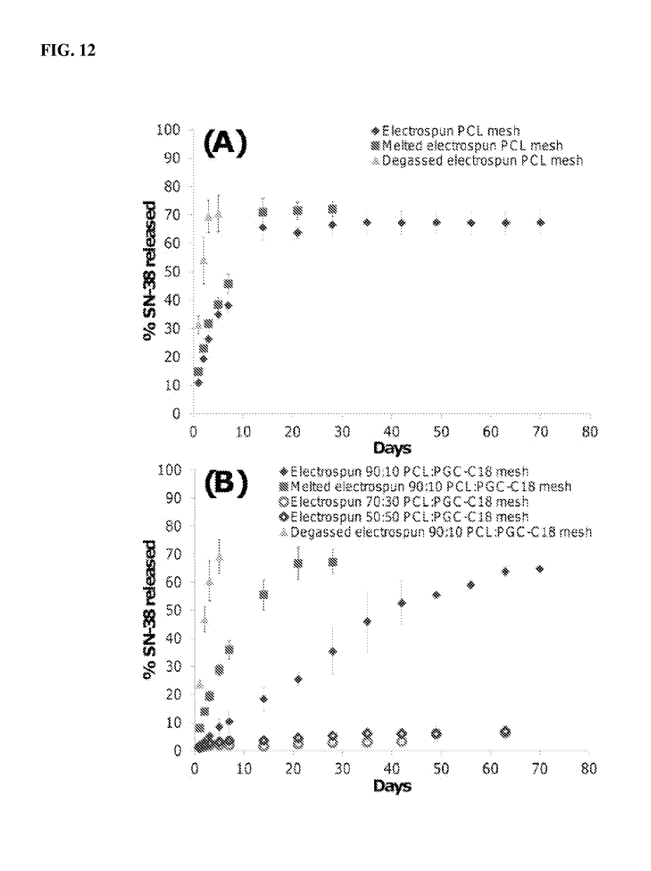

Meshes were prepared similar to Example 1, where 7-ethyl-10-hydroxycamptothecin (SN-38) was added to the electrospinning solution to encapsulate the drug within the fibers. SN-38 loaded electrospun meshes and melted electrospun meshes were kept completely submerged in pH 7.4 phosphate buffered saline (PBS) during release, and release media was changed regularly to maintain sink conditions for the drug (<10% drug solubility). The release profile of porous electrospun meshes for PCL, 10% PGC-C18 doped PCL, 30% PGC-C18 doped PCL, and 50% PGC-C18 doped PCL compared to smooth melted electrospun surfaces. Electrospun PCL meshes and melted PCL meshes show similar release rates, whereas the 10% doped PGC-C18 electrospun meshes significantly slowed drug release compared to their melt control (FIG. 12A). The melted 10% PGC-C18 doped PCL meshes stop releasing SN-38 by 28 days, whereas electrospun meshes continue to release out to 70 days. The electrospun 10% PGC-C18 doped PCL mesh (i.e., the more porous and high surface area material) releases drug more slowly (FIG. 12B). These results are consistent with the observation that the 10% PGC-C18 doped PCL electrospun mesh is in the metastable-Cassie state—the material starts with air entrapped within the porous structure, and with time air is slowly displaced to create more area at the water-surface interface for SN-38 to be released. This finding prompted the inventors to evaluate a higher PGC-C18 doping concentration to determine if an electrospun mesh with a more stable air layer could further slow release. The 30% and 50% PGC-C18 doped electrospun meshes showed only ≈10% SN-38 release over 9 weeks. For comparison, melted meshes show water at the surface, consistent with the lack of porosity within the structure. Finally, an electrospun mesh that has been degassed via sonication releases its drug at a significantly faster rate. 70% of the entrapped SN38 is released within 7 days from sonicated 10% doped PGC-C18 PCL electrospun meshes, compared to 70 days of release for the native electrospun mesh.

### Example 6B

### SN-38 Loading Affects Drug Release Rate from Undoped Poly(Caprolactone) Meshes and Poly(Caprolactone) Meshes with Hydrophobic Polymer Dopant

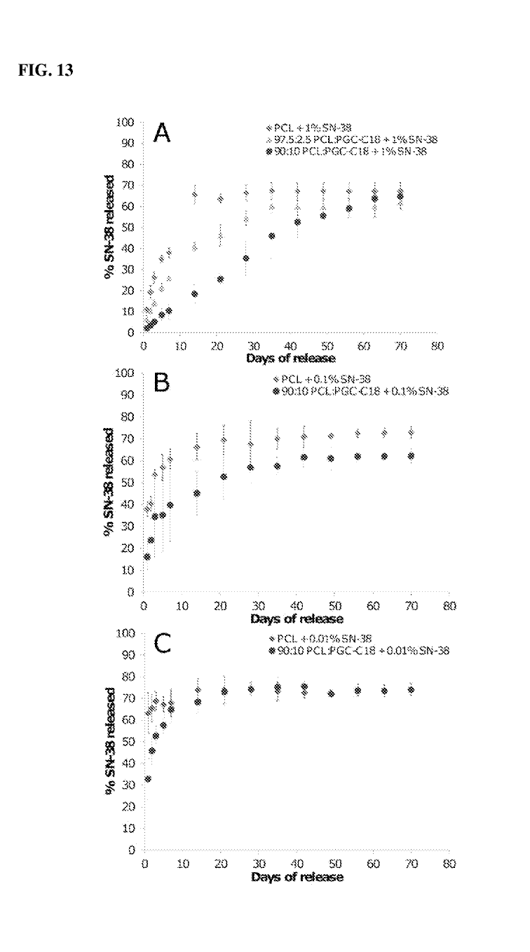

Meshes were prepared similar to Example 1. PCL electrospun meshes have an apparent contact angle of 121°, and doping 10% PGC-C18 increases the apparent contact angle to 143°, indicating slower drug release will occur from meshes with 10% of PGC-C18 doping. Release data for different concentrations of SN-38 indicates that this is the case with dramatically different release profiles for PCL meshes and PCL meshes doped with 10% PGC-C18 (FIG. 13). SN-38 release is significantly slower for all three drug concentrations when doping 10% PGC-C18 into PCL meshes. Using 1 wt % SN-38 loading as an example for this phenomenon, undoped PCL meshes have been shown to quickly release their drug payload by 14 days whereas SN-38 is linearly released for 70 days without any significant burst release when doping PCL meshes with 10% PGC-C18. To further show that drug release can be tailored via PGC-C18 doping, an additional mesh was run with 2.5% PGC-C18 doping with 1 wt % SN-38 loading (apparent CA=128°). Doping with 2.5% PGC-C18 showed an intermediate release rate between PCL and 10% PGC-C18 doping, with extension of SN-38 release for an additional 10 days compared to PCL alone. PCL meshes doped with 30% and 50% PGC-C18 significantly slowed release compared to 10% PGC-C18 doping, with <10% SN-38 release at 9 weeks. Decreasing the amount of drug loading to 0.1 wt % and 0.01 wt % increases both the release rate and burst release for PCL and PCL doped with 10% PGC-C18. The difference in SN-38 partitioning seen with confocal microscopy is consistent with this change in release. Loading with 0.1 wt % and 0.01 wt % SN-38 leads to surface segregation within individual electrospun fibers, leading to a smaller distance for drug to diffuse into the release media from fibers, thus accelerating drug release.

### Example 6C

### Anti-Proliferative Efficacy of Polymer Meshes with and without a Hydrophobic Doping Agent Against Cancer Cell Lines

Drug-loaded non-woven polymer meshes and blends were prepared from polycaprolactone with or without the inclusion of 10 w/w % poly(glycerol monostearate-co-caprolactone). The meshes contained either 0.01, 0.1, or 1.0 w/w % drug (SN38 or CPT-11). The camptothecin release from these meshes was tested using a cell viability assay following 24 hour treatments with camptothecin-loaded or unloaded meshes. LLC lung cancer cells or HT29 colorectal cancer cells were maintained in their respective serum positive media (SPM) containing 10% fetal bovine serum and penicillin/streptomycin (100 U/100 ug/mL). Individual camptothecin-loaded and unloaded meshes were placed in permeable filter supports (Polyester membrane insert, 3.0 μm pore size; Corning Incorporated, Corning, N.Y.) and maintained in SPM. Medium was changed periodically to ensure continued drug release. All cell cultures and meshes in holding wells were maintained in a humidified atmosphere at 37° C. and 5% CO2. At designated time points, subconfluent cells were harvested and seeded on 12-well plates at 30,000 cells/well in SPM. After 24 hours, filter supports and meshes were transferred from holding wells to the wells containing tumor cells in 2 mL of fresh SPM. After 24 hours of co-incubation with tumor cells, films were returned to holding wells in fresh SPM until the subsequent time point. Five days after treatment, tumor cell viability was measured using a colorimetric MTT (3-(4,5-dimethyl-2-thiazolyl)-2,5-diphenyltetrazolium bromide) cell proliferation assay (Sigma, St. Louis, Mo.). Cell viability was calculated as the percentage of the positive control absorbance for each cell line at each time point. SN38-loaded meshes demonstrated prolonged anti-proliferative efficacy for greater many weeks, while CPT-11 were effective over shorter durations.

### Example 6D

### CPT-11 Release from Poly(Caprolactone) Non-Woven Meshes with and without a Hydrophobic Doping Agent

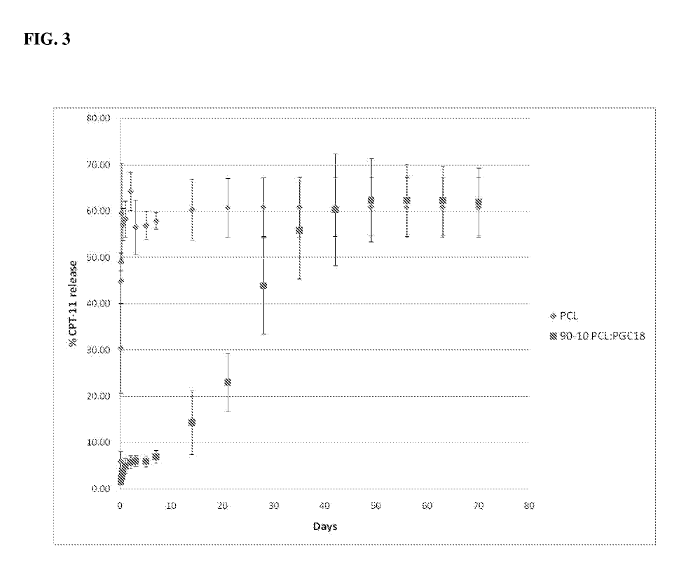

Meshes were prepared similar to Example 1, where 1% CPT-11 was loaded into each mesh chemistry. Release of 1 wt % CPT-11 loaded meshes was significantly delayed from PCL meshes doped with 10% PGC-C18 (FIG. 3). Undoped PCL meshes release CPT-11 very quickly over a few days, whereas the addition of 10% PGC-C18 slows CPT-11 release dramatically, with an initial burst release of ≈5% and a gradual release of drug out to 50 days. The release profile seen with 10% of PGC-C18 indicates that CPT-11 localized at the surface of the meshes is released very quickly, accounting for the burst, and is followed by slow, sustained release as water infiltrates into the meshes. CPT-11 from both PCL and PCL doped with 10% PGC-C18 is released more quickly than equitable meshes loaded with SN-38 due to the increased solubility of CPT-11 in the release media. This finding makes intuitive sense since the prodrug CPT-11 was selected for systemic delivery due to its increased solubility over SN-38. Release with all drug/dose/polymer formulations reached a maximum of ≈72% of total encapsulated drug. The remaining drug which was not released was shown to still be encapsulated in meshes as confirmed by HPLC. This is a common phenomenon with surface eroding polymers such as PCL, where a percentage of the drug is trapped within the polymer matrix until bulk matrix degradation occurs.

### Example 7

### Prevention of Tumor Recurrence Using Camptothecin-Loaded Meshes in a Lung Cancer Recurrence Model

Female C57BL/6 mice at six to eight weeks of age were obtained from Jackson Laboratories (Bar Harbor, Me.). A primary tumor was induced by subcutaneous injection of 7.5×105 Lewis Lung Carcinoma (LLC) cells (in 0.2 mL PBS) on the dorsum of Female C57BL/6 via a 27-gauge needle attached to a 1 mL syringe. This tumor dose effectively results in rapidly progressive tumor within 2 weeks. Tumor volume was estimated by the formula (length×width×height×Pi)/6, and the primary tumor was surgically removed when the tumor reached 300 mm². This size was chosen as the majority of animals will develop locally recurrent tumor despite aggressive surgical resection if no additional therapeutic intervention is performed to prevent recurrent disease. Unloaded or camptothecin-loaded meshes (1.0×0.8 cm; 10% w/w), similar to those described in Example 3, were implanted with the polymer abutting the area of surgical resection. The four corners of the mesh were sutured to the superficial fascia in order to secure the position of the strip and the skin incision is closed with 5-0 polypropylene sutures. Tumor controls were utilized where no additional therapy was given following surgical resection in order to establish the incidence of recurrence in these experiments. The resulting data indicate that camptothecin-loaded polymer blend meshes incorporated at the surgical margin, can afford enhanced local drug delivery aimed at preventing the growth of occult disease present following parenchyma-sparing surgery, and offer the means to decrease local recurrence rates in patients with stage I-IIIa lung cancer in the future.

### Example 8

### Removal of Air Layer with Ethanol from Poly(Caprolactone) Non-Woven Meshes with and without a Hydrophobic Doping Agent Causes Expedited Release

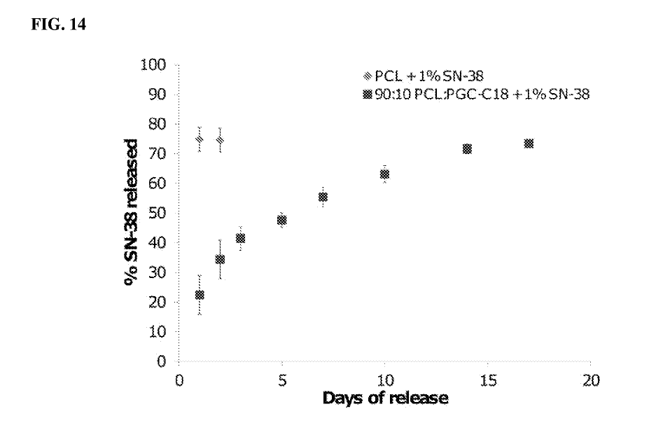

Meshes were prepared similar to Example 1. An additional release study was performed with 1 wt % SN-38 loaded electrospun meshes to determine how drug release rates change when air is removed and no longer controls drug release. Meshes were quickly dipped in ethanol and moved to PBS release buffer. Ethanol has a low surface tension (22.4 mN/m) and wets all superhydrophobic electrospun meshes regardless of PGC-C18 doping. Without the air entrapped within electrospun meshes, a significantly different release profile is observed. PCL meshes release the entire drug payload within 1 day compared to 14 days from native electrospun meshes where air is still entrapped (FIG. 14 vs. FIG. 13). PCL meshes with 10% PGC-C18 doping release all drug within 17 days rather than 70 days. A large burst of SN-38 is released in both mesh types, where the large concentration of surface drug seen with confocal imaging quickly partitions into solution. With slow penetration of water into native electrospun meshes, and the displacement of entrapped air, the release of surface drug is normally averaged out over many days. However, with ethanol wetting, the mesh is degassed and all surface drug is released as a bolus within 2 days. With 10% PGC-C18 doping, linear drug release from electrospun fibers is seen for 10 days after the initial burst. Overall, this study reconfirms the effectiveness of PGC-C18 to slow the release of a hydrophobic drug, as well as the importance of air in slowing the release from the superhydrophobic meshes.

### Example 9

### Computed Tomography Scans Show Presence of Entrapped Air Within Poly(Caprolactone) Non-Woven Meshes with and without a Hydrophobic Doping Agent

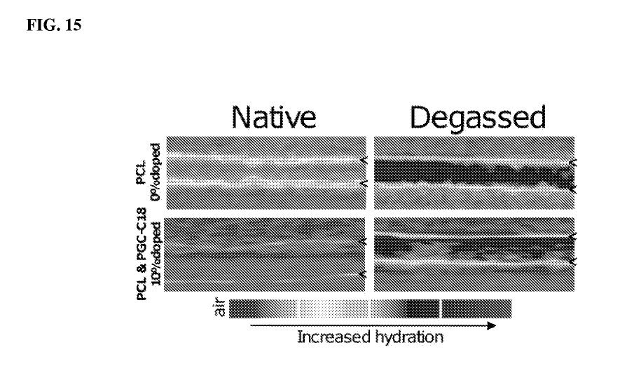

CT scans of native electrospun and degassed electrospun meshes with 0 or 10% PGC-C18 doping after incubation with the contrast agent Hexabrix for 2 hours (FIG. 15). Degassed meshes exhibit full water penetration, while native and melted meshes (not shown) show only a low surface concentration of water. Tic marks define the top and bottom boundaries of the meshes.

### Example 10

### Clinical Ultrasound Scans Demonstrate Entrapped Air within Poly(Caprolactone) and Doped Poly(Caprolactone) Meshes

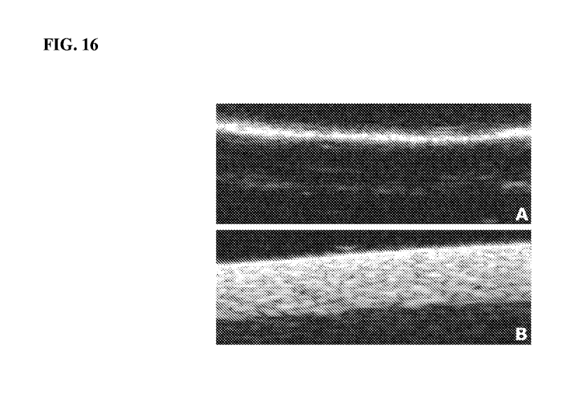

A VisualSonics™ Inc ultrasound imaging device with a 55 MHz scanhead was used to image both native and degassed electrospun meshes. Native electrospun meshes showed no water penetration after 2 hours and, as expected, the entrapped air results in an anechoic shadow within the bulk of the mesh appearing dark on ultrasound imaging with a bright edge (FIG. 16). This is in marked contrast to degassed electrospun meshes, where water infiltrates the entire electrospun mesh structure, lowers the degree of ultrasound reflection and allows the entirety of the mesh to be visualized as an echogenic mesh. This further confirms that entrapped air is present in the superhydrophobic meshes and can serve as a degradable component within the materials to slow drug release.

This ability to visualize the mesh “remotely” with ultrasound will enable non-invasive monitoring of not just drug release correlating with the “wetting of the mesh”, but also serve as a marker for the surgical site after implantation. For example, the mesh can demarcate the area of the anastomosis to quickly identify surgical complications (i.e. anastomotic stricture or fluid collection) via transabdominal ultrasound. Currently, normal bowel gas can make the stapled anastomosis difficult to visualize and oral contrast with radiographic imaging via CT scanning is often required to rule out anastomotic complications, resulting in increased costs and radiation exposure for the patient. Occasionally a rectal ultrasound is used to look for peri-anastomic fluid, but the need for a distending balloon within the rectum at the area of the anastomosis for good image quality is of significant concern in the clinical setting of a recent anastomosis. In addition to identifying surgical complications, the presence of the mesh at the anastomosis can also identify the area at greatest risk for recurrent disease and allow focused post-surgical surveillance of this area.

### Example 11

### Long Term 3D Superhydrophobicity within Poly(Caprolactone) and Doped Poly(Caprolactone) Meshes is Affected by Hydrophobic Doping Concentration

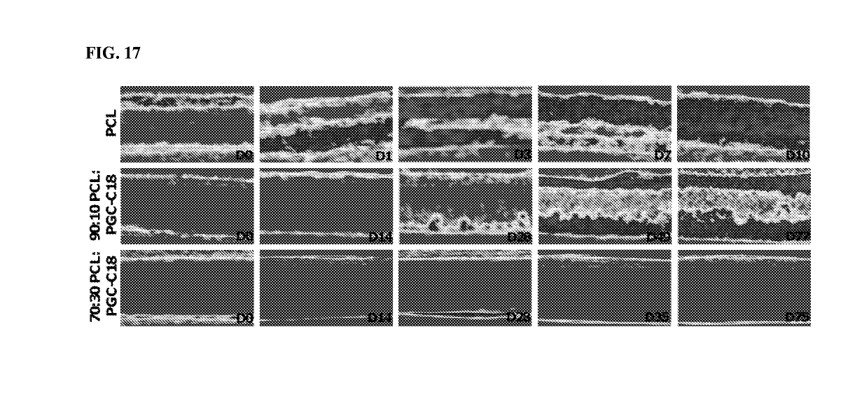

The stability of the Cassie state (nonwetting state) of these superhydrophobic mesh formulations was confirmed by directly measuring the infiltration of water with a series of 3D superhydrophobic materials, including PCL (7.7 μm, 123°), 10% PGC-C18 doped PCL (7.2 μm, 142°), 30% PGC-C18 doped PCL (2.4 μm, 150°), and 50% PGC-C18 doped PCL (169 nm, 168°) using a number of physio-chemical techniques. These formulations were selected to span a range of 3D superhydrophobicities, fiber sizes, and surface chemistries. Quantitative X-ray computed tomography (μCT) was used to measure the rate and depth of water infiltration. A 3:1 water-ioxaglate solution (an anionic iodinated CT contrast agent) was incubated with the superhydrophobic electrospun meshes and the depth/rate at which water penetrated into the mesh was determined from the CT signal as the contrast agent solution wetted the mesh. This study is shown pictorially, where the progression of water infiltration is tracked through the cross section of a representative mesh for PCL, PCL with 10% PGC-C18, and PCL with 30% PGC-C18 (FIG. 17). The infiltration rate into superhydrophobic meshes was then plotted, where PCL is fully wetted within 10 days and all air has been displaced within the meshes.

Approximately 80% of the air is displaced within 2 days, followed by the removal of the remaining 20% of air in the following 8 days. The weakly metastable state of entrapped air within PCL allows eventual removal of all air. The more hydrophobic electrospun PCL meshes with 10% PGC-C18 doping are also metastable, but show a much slower, sustained displacement of air, where an average of 52% of air has been displaced by 77 days. Finally, PCL with 30% PGC-C18 showed a stable air layer over the length of the study, with only 1% of air displaced over 35 days and water is only observed at the outer superhydrophobic material surface. A follow up scan of 30% PGC-C18 doped PCL electrospun meshes after 75 days of incubation showed <4% of the meshes had been infiltrated, demonstrating prolonged underwater stability of the Cassie state.

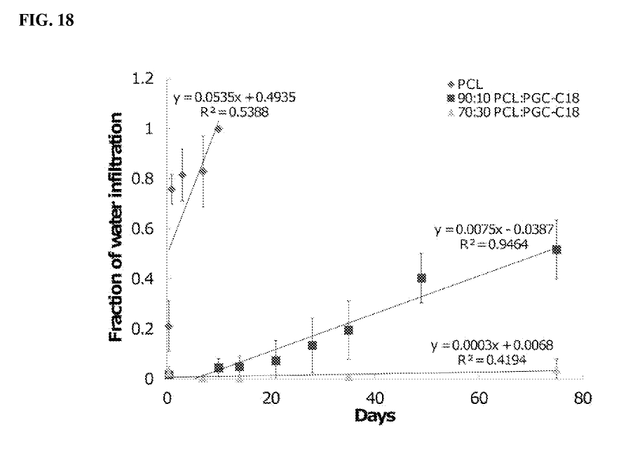

Linear regression of water infiltration into PCL, PCL with 10% PGC-C18, and PCL with 30% PGC-C18 shows infiltration rates of 13.5, 2, and 0.07 μm/day/side, respectively, which corresponds to 5.4%, 0.8%, and 0.03% total infiltration (both sides) per day for 500 μm meshes (FIG. 18). Differences in infiltration rates were statistically significant using an analysis of covariance (ANCOVA) (p<0.01). The PCL mesh with 50% PGC-C18 doping was not studied since water infiltration are expected to be even slower as it is more hydrophobic than the 30% PGC-C18 containing meshes. The results of this study indicate that both PCL and PCL with 10% PGC-C18 doping are in the metastable state where water infiltrates the mesh and displaces entrapped air, and that 30% PGC-C18 doping leads to a stable Cassie state where the air layer is permanently maintained.

### Example 12

### An Exemplary Mechanism of Drug-Eluting Superhydrophobic Meshes

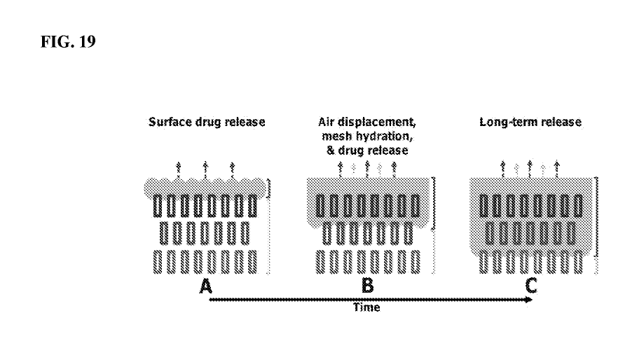

Without wishing to be bound by theory, FIG. 19 depicts an exemplary mechanism of a drug-eluting 3D superhydrophobic material in a metastable Cassie state. Over time, water slowly displaces air content from the material with the transition from the metastable Cassie state to the stable Wenzel state. If treated as iterative surfaces, water slowly penetrates each individual surface over time enabling prolonged drug release.

### Example 13

### Stearate Modification/Modification Produces Increases Contact Angle and Apparent Contact Angle for Poly(Caprolactone) and Doped Poly(Caprolactone) Meshes and Films

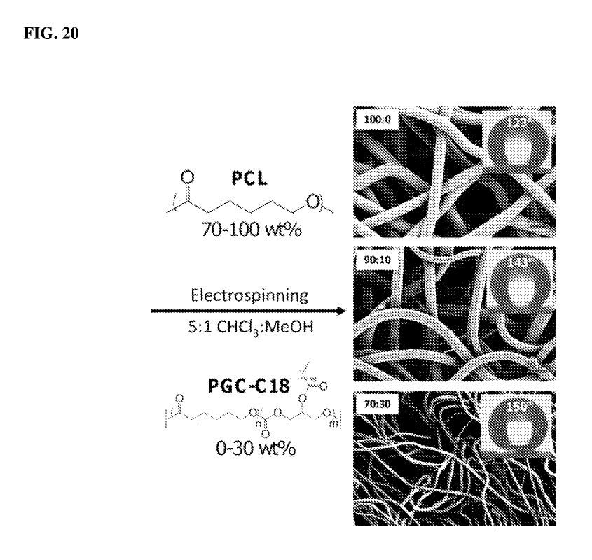

The 3D superhydrophobic materials described herein were prepared from electrospun poly(ε-caprolactone) (PCL) and poly(glycerol monostearate-co-ε-caprolactone) (PGC-C18), where PGC-C18 is doped into PCL in different proportions to tailor the overall superhydrophobic state. PGC is a copolymer of caproic acid and glycerol (4:1), where the glycerol subunit can be modified with various pendant groups to impart functionality or alter the hydrophilicity/hydrophobicity of the polymer. In this study, stearic acid was added to produce a hydrophobic polymer (PGC-C18) to slow, or prevent, water penetration into the mesh. The presence of a large number of flexible, hydrophobic stearate (—O(O)C(CH₂)₁₆CH₃) pendant groups leads to a decrease in the surface energy of the doped meshes. Undoped PCL electrospun meshes are modestly hydrophobic with an apparent contact angle of 123°. Adding PGC-C18 increases the apparent contact angle of the electrospun meshes to 150° with 30 wt % PGC-C18 doping (20 wt/v % electrospinning solution) (FIG. 20). The stearate modification is required for the superhydrophobic effect, since electrospun PCL with doped PGC-OH, which lacks the stearate group, has no apparent contact angle (ACA=0°) and wets with the application of a water droplet. The molecular weight of PGC-C18 is much lower than the PCL used in these studies (20 kDa vs. 70-90 kDa). Therefore, increasing the amount of PGC-C18 also leads to a decrease in electrospinning solution viscosity and subsequent decrease in fiber size. With 10% PGC-C18 doping there is a modest decrease in fiber size compared to PCL (7.7 μm vs. 7.2 μm), and a greater decrease with 30% PGC-C18 doping (2.46 μm).

### Example 14

### Hydrophobic Doping Concentration and Surface Roughness Modify Superhydrophobic State of Poly(Caprolactone) and Doped Poly(Caprolactone) Meshes

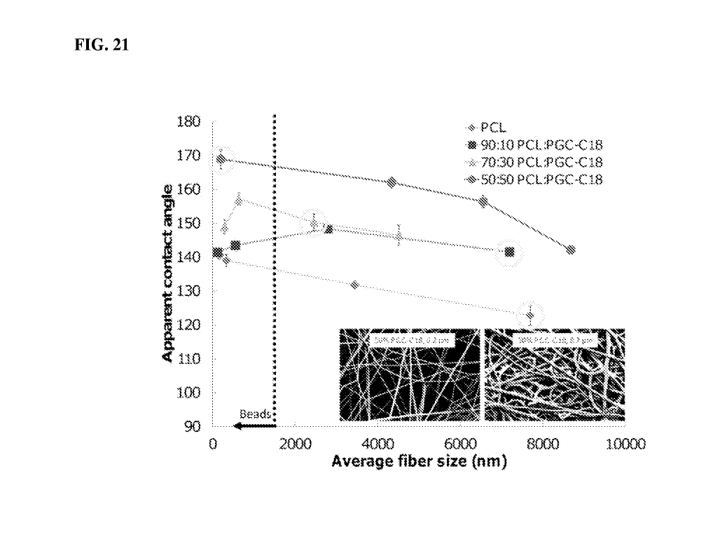

Changes in polymer hydrophobicity and electrospun fiber size contribute to 3D superhydrophobicity, as both the surface energy and the proportion of air exposed at the surface influence the overall superhydrophobic state. In order to observe changes in polymer surface energy, the contact angles of flat PCL-PGC-C18 blended surfaces were compared. Solvent cast films prepared from PCL, PCL with 10% PGC-C18, and PCL with 30% PGC-C18 have contact angles of 83°, 109°, and 111°, respectively, where an increase in the hydrophobic polymer dopant PGC-C18 to PCL leads to a larger contact angle, and thus a lower surface energy. All of the contact angles for the cast films are lower than the contact angle values for the corresponding meshes (121°, 143°, 150°, respectively), confirming the presence of entrapped air at the surface and the property of superhydrophobicity. Next, the fiber size of the electrospun meshes was altered to study the effect of surface roughness and surface fill fraction on superhydrophobicity by modifying the electrospinning solution and processing parameters. FIG. 21 shows the resultant apparent contact angle for the three superhydrophobic mesh chemistries as a function of changes in fiber size/surface roughness. PCL electrospun meshes were produced with fiber sizes ranging from 166 nm to 7.7 μm. The smallest fibers lead to an apparent contact angle of 141°, whereas the largest fiber had an apparent contact angle of 123°. This result was expected, as it is known that decreasing the surface fill fraction, or reducing the amount of polymer exposed at a given surface, results in a higher apparent contact angle. The 10% PGC-C18 and 30% PGC-C18 doped PCL meshes initially follow this trend (where a decrease in fiber size leads to an increase in apparent contact angle), but exhibit an eventual decrease with continued fiber size reduction. Specifically, the 10% PGC-C18 doped PCL meshes reached a maximum apparent contact angle of 148° with a fiber size of 2.7 μm, followed by a decrease to 142° with 123 nm fibers. The 30% PGC-C18 meshes reached a maximum apparent contact angle of 157° with 641 nm fibers, and the apparent contact angle decreased to 149.3° for the 296 nm fibers.

Without wishing to be bound by theory, one possible explanation for this increase and subsequent decrease in apparent contact angle with fiber size reduction is that the PGC-C18 is partitioning to the surface of the fibers. Polymers of different compositions are known to phase separate, both within the bulk and at the surface of the material. Differential scanning calorimetry experiments indicate that phase separation within the bulk of the electrospun meshes does not occur, as PCL and PGC-C18 are sufficiently chemically similar. However, phase separation at the surface can occur to reduce the surface energy at an interface, which is commonly observed with polymer blends both on flat and textured material surfaces. These results indicate that the hydrophobic soft chain stearate segment of the copolymer preferentially partitions to the material surface such that a significant hydrophobic effect is observed with modest additions of PGC-C18.

With large fibers, the exposed surface becomes easily saturated with these highly hydrophobic pendant groups. However, as the surface area to volume ratio becomes larger, as a consequence of a decrease in fiber size, the same PGC-C18 content is insufficient to generate the same hydrophobic effect. With 10% PGC-C18, superhydrophobicity begins to decrease with fiber sizes below 2.8 μm, with the underlying PCL bulk material contributing to the surface composition. Increasing the doping to 30% PGC-C18 in PCL meshes provides 3-times the amount of stearate groups to functionalize this larger surface area, but there is still an eventual decrease in the apparent contact angle at smaller fiber size. Traditionally a decrease in surface fill fraction from smaller fibers leads to a higher apparent contact angle, as in seen in single phase PCL meshes. However, the addition of PGC-C18 presents a competing mechanism, where higher concentrations of PGC-C18 are required to cover the increased surface area produced with small fibers to maintain the superhydrophobic effect. PCL meshes with 50% PGC-C18 doping were fabricated to confirm these competing effects, where an increase in apparent contact angle is shown for all fiber sizes. The largest fibers produced (8.7 μm) have an apparent contact angle of 142°, while the smallest fibers (206 nm) have an apparent contact angle of 169°, demonstrating that superhydrophobicity continues to increase with a reduction in fiber size.

### Example 15

### Surfactancy Modifies the Superhydrophobic Effect of Poly(Caprolactone) and Doped Poly(Caprolactone) Meshes

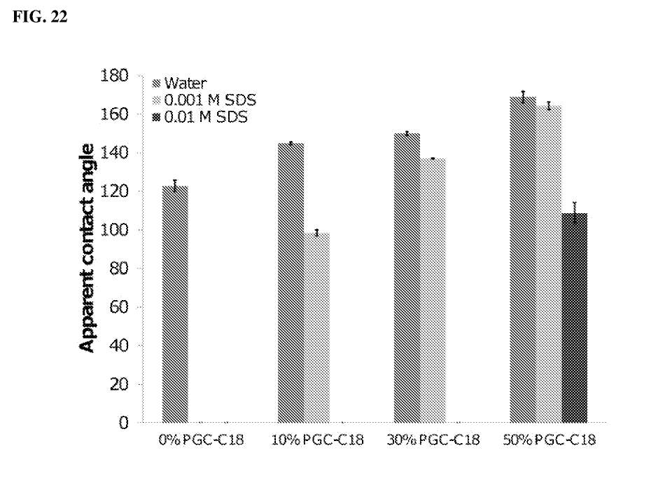

The Cassie state of wetting that defines superhydrophobicity is a result of an interaction between a low surface energy material and a high surface tension liquid (i.e., PCL-PGC-C18 meshes and water). Air is maintained at the material-liquid surface, reducing formation of a high energy interface. The superhydrophobic characteristics of a surface are decreased or removed with changes in the surface energy of either phase, either with an increase in the surface energy of the material surface or a decrease in the surface tension of the liquid. The use of surfactants is one method to modulate the energy of either/both phase(s), where surfactants decrease the surface tension of water by lowering the energy of the air-water interface, or alternatively, the hydrophobic domains of the surfactant can bind the material surface to increase the energy of the surface. The effect that a particular surfactant has on water surface tension and material surface energy depends on both the surfactant structure as well as the extent of adsorption. Two common surfactants, sodium dodecyl sulfate (SDS) and polysorbate 20, were used to determine how the superhydrophobic characteristics of the electrospun meshes are modified. SDS was used at two different concentrations (0.001M, 0.01M), where the SDS was added to the probing solution for apparent contact angle measurements. By adding SDS to the probing medium, the effect of a decrease in water surface tension was assessed since insufficient time was provided for SDS to adsorb to the mesh surface (FIG. 22). The difference (Δ) in apparent contact angle between water and SDS containing solutions was statistically significant when comparing any pair of mesh chemistries (i.e, PCL vs. PCL with 10% PGC-C18 with 0.001 M SDS; p-value<0.001).

Application of droplets containing 0.001 M (ST≈63 mN/m) to electrospun PCL meshes resulted in no apparent contact angle (i.e., complete wetting), compared to 123° for water alone. Application of 0.001 M SDS solutions to electrospun PCL meshes with 10%, 30%, and 50% PGC-C18 resulted in lower apparent contact angles compared to water, where increased PGC-C18 showed less of a reduction in contact angle (Δ47°, 13°, and 6° respectively). A 10-fold increase in the SDS concentration (0.01 M; ST≈35 mN/m) provided a sufficient drop in surface tension to fully wet the 10% and 30% PGC-C18 doped PCL meshes. The 50% PGC-C18 containing meshes were not completely wetted, though a significant drop in the apparent contact angle to 109° (Δ60° from water) was observed.

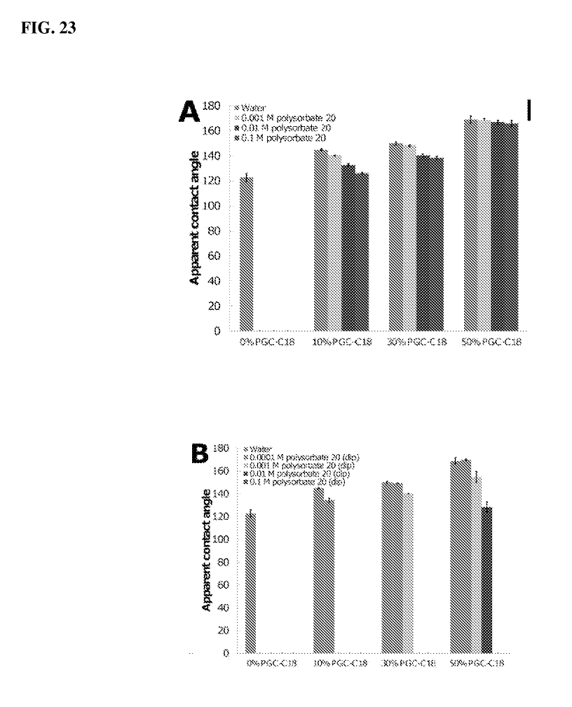

The effect of polysorbate 20 on mesh superhydrophobicity was examined where 1) surfactant was added to the water probe in a manner similar to the SDS experiments, and 2) by soaking electrospun meshes in polysorbate 20 solutions (FIG. 23). These experiments studied how a decrease in surface tension alters the superhydrophobic characteristics of the meshes using a second surfactant, and examined how long term incubation allows adsorption of surfactant to a mesh surface, leading to an increase in surface energy. The effect of polysorbate 20 concentration on apparent contact angle when added to the water probe was performed at three concentrations (0.001 M, 0.01 M, 0.1 M). Surface tensions of all solutions are ≈40 mN/m, and all concentrations are above the critical aggregation concentration for polysorbate 20. The difference (Δ) in apparent contact angle between water and polysorbate 20 containing solution was statistically significant when comparing any pair of mesh chemistries (i.e., PCL vs. PCL with 10% PGC-C18 with 0.1 M polysorbate 20; p-value<0.01), except between 30% PGC-C18 and 50% PGC-C18 meshes with 0.001 M solutions, and 10% PGC-C18 and 30% PGC-C18 meshes with 0.01 M solutions. When probing PCL mesh surfaces, all concentrations of polysorbate 20 led to immediate penetration of water, and no apparent contact angle was observed. Adding 10%, 30%, and 50% PGC-C18 to PCL electrospun meshes stabilized the entrapped air layer, and only showed a modest decrease in apparent contact angle for all polysorbate 20 concentrations, where even the largest polysorbate 20 concentration (0.1 M) was not sufficient to wet the meshes (Δ19°, 11°, 3°, respectively, for 0.1 M solutions).

Superhydrophobic meshes were then incubated in polysorbate 20 solutions (0.0001-0.1 M) for 24 hours, after which samples were air dried and probed using pure water. In this procedure, the polysorbate 20 had adsorbed to the surface of the meshes. The resulting polysorbate 20 treated meshes possessed a significantly reduced apparent contact angle. PCL electrospun meshes wetted at all polysorbate 20 concentrations. Adding 10% PGC-C18 made the entrapped air layer slightly more robust than PCL meshes, but only formed an apparent contact angle with the lowest polysorbate 20 concentration used for incubation (Δ10° for 0.0001 M solution). The surfaces were incrementally more robust with addition of 30% PGC-C18, and the entrapped air layer was stable at the two lowest concentrations selected (Δ1° for 0.001 M; Δ10° for 0.0001 M). PCL meshes with 50% PGC-C18 formed an apparent contact angle with all but the highest polysorbate 20 solution (0.1 M), with apparent contact angle changes of 0°, 14°, 40° for solutions with 0.0001 M, 0.001 M, and 0.01M polysorbate 20. The difference (Δ) in apparent contact angle between meshes with and without polysorbate 20 incubation was statistically significant when comparing any pair of superhydrophobic mesh chemistries (i.e., PCL vs. PCL with 50% PGC-C18 with 0.01 M polysorbate 20; p-value<0.01), except between 30% PGC-C18 and 50% PGC-C18 meshes with 0.001 M polysorbate 20 incubation.

### Example 16

### Solvents of Different Surface Tension Modifies the Superhydrophobic Effect on Poly(Caprolactone) without a Hydrophobic Dopant and Poly(Caprolactone) with a Hydrophobic Dopant

A “modified Zisman curve” for each of the superhydrophobic mesh chemistries was next completed. Zisman curves are traditionally used to probe flat surfaces, where solvents of different surface tensions are used to identify the critical surface tension in which there is no observable contact angle. This method was adapted to characterize the meshes and used solvents of different surface tension to probe the mesh surface, ranging from water (72 mN/m) to ethanol (22 mN/m). In this experiment, the critical surface tension corresponds to an apparent contact angle of 0°, or one where there is no barrier to immediately absorb into the electrospun material (FIG. 16). PCL electrospun meshes were determined to have a critical surface tension of 57 mN/m, where only a small decrease from the surface tension of water (Δ15 mN/m) resulted in no barrier for wetting. The entrapped air layer was more robust for PGC-C18 containing meshes as compared to PCL alone. PCL with 10% PGC-C18 formed an apparent contact angle with solvents with surface tensions as low as 44 mN/m, and PCL with 30% PGC-C18 formed an apparent contact angle until 39 mN/m. PCL with 50% PGC-C18 formed an apparent contact angle with solvents with surface tensions as low as 33 mN/m. Solvents exposed to these materials below these surface tension values resulted in complete wetting, where, for example, ethanol treatment results in complete wetting for all mesh types. A best-fit line was then calculated, which approximated the surface tension required for a 50% reduction in apparent contact angle from pure water for each superhydrophobic mesh chemistry. These values were found to be 60.6, 40, 34.7, 30 mN/m for PCL, PCL with 10% PGC-C18, PCL with 30% PGC-C18, and PCL with 50% PGC-C18, respectively, which closely match surface tension values that lead to complete infiltration (57, 39, 33, 27.6 mN/m). The small difference in surface tension for a 50% reduction in apparent contact angle and complete infiltration shows that the stability of entrapped air, or the stability of the superhydrophobic state drops quickly as the critical surface tension for complete infiltration is approached. Critical surface tension values for complete wetting found in this study are also consistent with previous surfactant studies by the inventors, where PCL meshes wetted below surface tensions of 63 mN/m, 10% and 30% PGC-C18 were shown to wet below 35 mN/m, and 50% PGC-C18 meshes did not wet in contact with solutions with surface tensions of 35 mN/m.

### Example 17

### Pressure Modifies the Superhydrophobic Effect on Poly(Caprolactone) without a Hydrophobic Dopant and Poly(Caprolactone) with a Hydrophobic Dopant

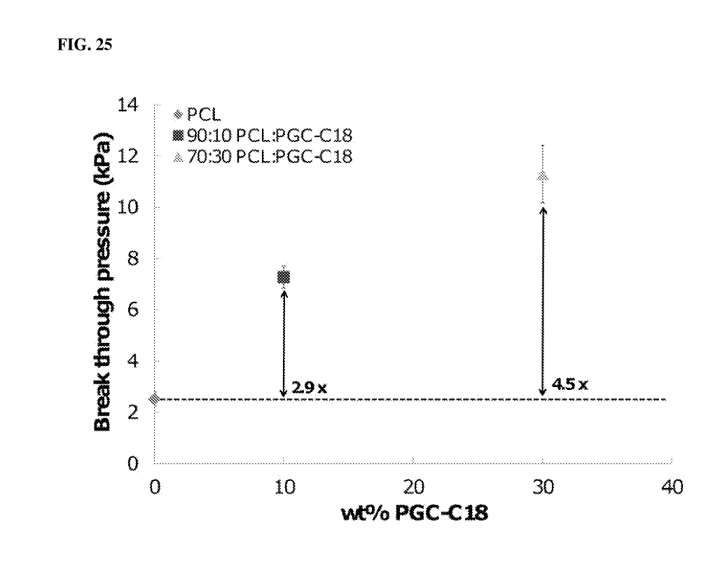

The pressures required for water to infiltrate into the superhydrophobic meshes using a filtration setup were studied. This water pressure value signifies the transition from a Cassie state (air entrapped) to a Wenzel state (air removed) for superhydrophobic materials. Water pressure applied to the electrospun meshes was increased to the point of initial wetting and breakthrough observed (FIG. 25). PCL electrospun meshes were easily wetted, with only 2.5 kPa of water pressure necessary to induce this transition. Increasing the PGC-C18 doping in PCL meshes raised the barrier to wetting with a 2.9-fold increase (7.3 kPa) in the pressure required to cause breakthrough with the 10% PGC-C18 doped meshes. A 4.5-fold increase (11.3 kPa) in the pressure was required for breakthrough over PCL meshes for 30% PGC-C18 doping. The difference in breakthrough pressure between any pair of meshes was statistically significant (p-value<0.001). Meshes with 50% PGC-C18 doping could not be evaluated, as the meshes tore before infiltration of water occurred.

### Example 18

### Serum Content Does Not Modify Superhydrophobicity in Undoped Poly(Caprolactone) and Poly(Caprolactone)

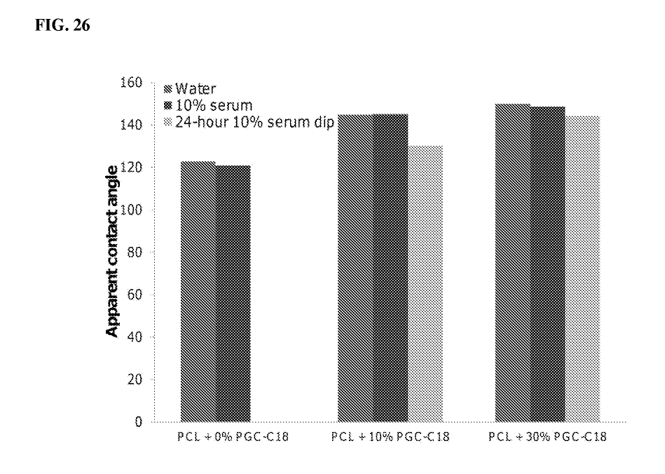

To test the effect of serum on the meshes, contact angle measurements were performed on three mesh chemistries (0, 10, 30% PGC-C18), where 10% serum was added to the applied droplet (FIG. 26). Minimal changes in contact angle were observed for all three meshes (<2°). Electrospun meshes were incubated in 10% serum containing PBS for 24 hours to determine if longer incubation times increased protein adsorption to promote wetting. No apparent contact angle was observed for the native PCL meshes, indicating significant amounts of protein adsorption occurred to promote wetting. With the 10% and 30% PGC-C18 doped PCL meshes, only a modest decrease (15° and 4°, respectively) in apparent contact angle was observed, showing that even in the presence of serum the entrapped air layer was present.

### Example 19

### SN-38 Loaded Electrospun Poly(Caprolactone) and Poly(Caprolactone) with Hydrophobic Dopant are Cytotoxic to Murine Lung Cancer Cell Line

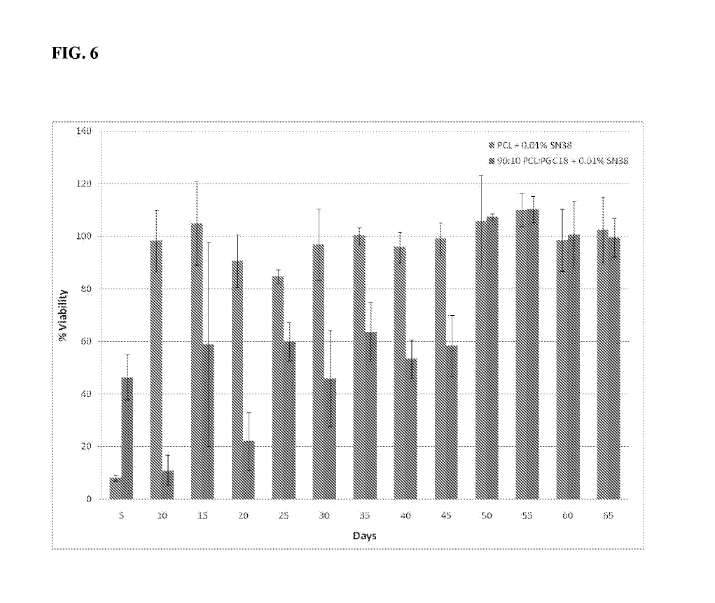

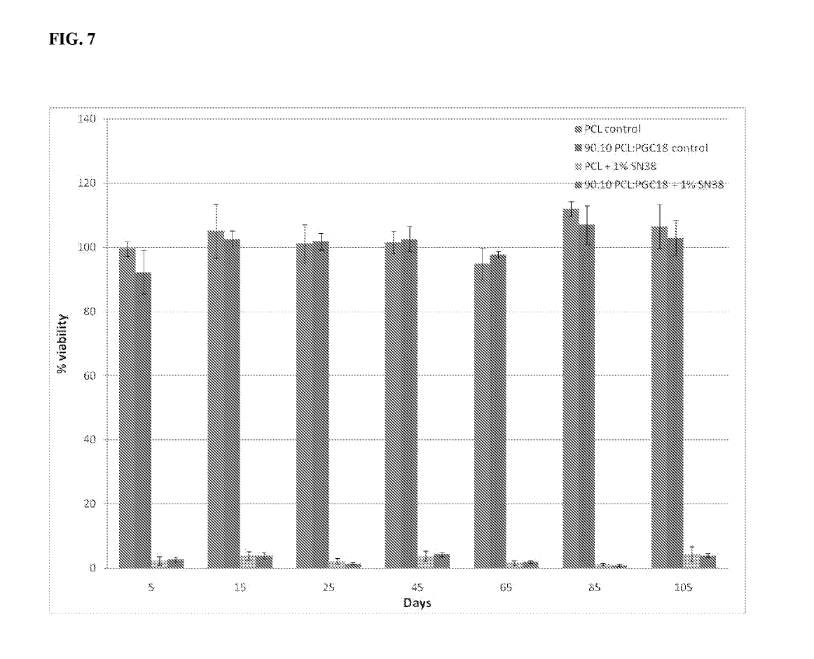

The SN-38 loaded meshes were incubated in serum containing media with Lewis Lung Carcinoma (LLC) cells (FIGS. 6 and 7). At a 1 wt % SN-38 concentration, both PCL and 10% PGC-C18 doped PCL meshes are cytotoxic to LLC cells for 90 days. No activity difference was seen between these meshes as even a very small amount of released SN-38 is cytotoxic due to the low IC-50 of SN-38 (≈8 ng/mL). Decreasing the SN-38 loading by 10-fold afforded a significant difference between the PCL and 10% PGC-C18 doped PCL meshes. The PCL meshes were cytotoxic for 25 days, whereas the 10% PGC-C18 doped PCL mesh was cytotoxic for 65 days. Unloaded meshes were not toxic to cells.

Drug-loaded non-woven polymer meshes and blends were prepared from polycaprolactone with or without the inclusion of 10 w/w % poly(glycerol monostearate-co-caprolactone). The meshes contained either 0.01, 0.1, or 1.0 w/w % drug (SN38 or CPT-11). The camptothecin release from these meshes was tested using a cell viability assay following 24 hour treatments with camptothecin-loaded or unloaded meshes. LLC lung cancer cells were maintained in their respective serum positive media (SPM) containing 10% fetal bovine serum and penicillin/streptomycin (100 U/100 ug/mL). Individual camptothecin-loaded and unloaded meshes were placed in permeable filter supports (Polyester membrane insert, 3.0 μm pore size; Corning Incorporated, Corning, N.Y.) and maintained in SPM. Medium was changed periodically to ensure continued drug release. All cell cultures and meshes in holding wells were maintained in a humidified atmosphere at 37° C. and 5% CO2. At designated time points, subconfluent cells were harvested and seeded on 12-well plates at 30,000 cells/well in SPM. After 24 hours, filter supports and meshes were transferred from holding wells to the wells containing tumor cells in 2 mL of fresh SPM. After 24 hours of co-incubation with tumor cells, films were returned to holding wells in fresh SPM until the subsequent time point. Five days after treatment, tumor cell viability was measured using a colorimetric MTT (3-(4,5-dimethyl-2-thiazolyl)-2,5-diphenyltetrazolium bromide) cell proliferation assay (Sigma, St. Louis, Mo.). Cell viability was calculated as the percentage of the positive control absorbance for each cell line at each time point. SN38-loaded meshes demonstrated prolonged anti-proliferative efficacy for greater many weeks, while CPT-11 were effective over shorter durations.

### Example 20

### SN-38 Loaded Electrospun Poly(Caprolactone) and Poly(Caprolactone) with Hydrophobic Dopant are Cytotoxic to Human Colorectal Cell Line

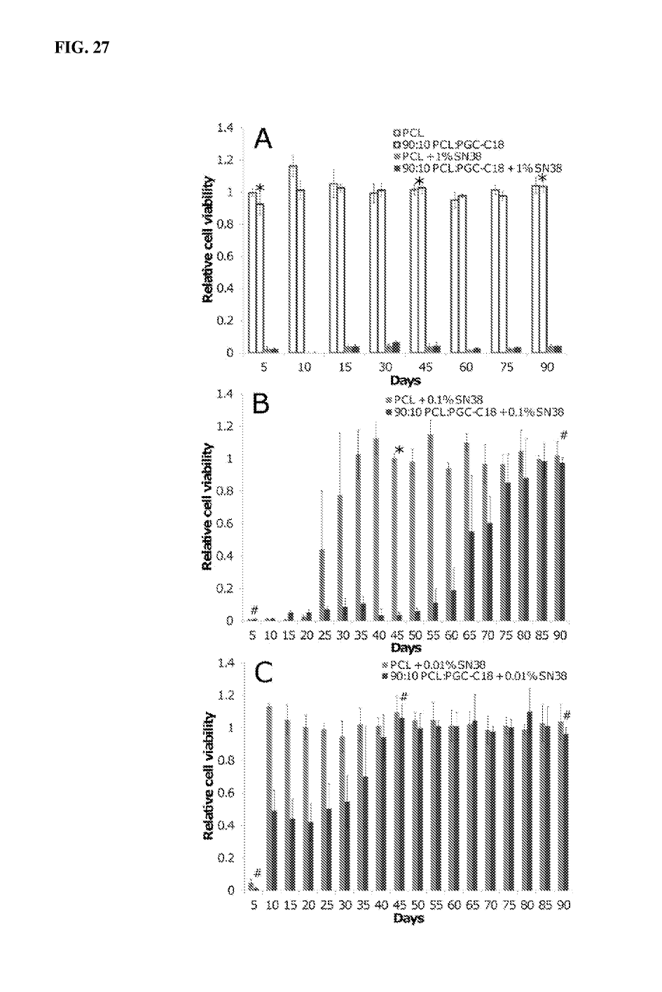

Superhydrophobic meshes were assessed in an in vitro cytotoxicity study against a colorectal cancer cell line (HT-29) (FIG. 27). Studies were performed by exposing cancer cells grown in monolayer cultures to meshes for a total of 24-hours while in serum. Meshes were subsequently removed and tumor cell viability was tested using a standard MTS assay 5 days later. Meshes were incubated in PBS between each time point with PBS replaced daily to ensure sink conditions for continued drug release and then moved to a new HT-29 cell monolayer for another 24 hour incubation period. The amount of SN-38 released from either PCL or PCL doped with 10% PGC-C18 loaded with 1 wt % SN-38 is sufficient to be cytotoxic to HT-29 for at least 90 days, whereas neither mesh was toxic without SN-38 loading. The continued cytotoxicity of PCL meshes, despite the limited duration of detected SN38 release (20 days from PCL meshes; 70 days from PCL doped with 10% PGC-C18 meshes), is likely due to the extremely low IC50 of SN-38 (3.4 ng/mL for HT-29 cells). This is supported by the very small amounts of SN-38 released over 70 days (>10 ng/day release at each time point) and detected in the lactone-carboxylate conversion study from both types of meshes when the initial SN-38 load was high.

Decreasing this SN-38 dose by 10-fold to 0.1% loading, or 100-fold to 0.01% loading, further supports this hypothesis with drug release falling off more rapidly and resulting in a more marked difference between PCL and PCL with 10% PGC-C18. The resultant cytotoxicity profile more closely mimicked the in vitro release profile of the respective formulations with 0.1 wt % SN-38 loaded PCL meshes no longer killing cancer cells by day 30, and SN-38 loaded 10% PGC-C18 doped PCL meshes demonstrated extended tumor cell cytotoxicity through day 60. With 0.01 wt % loading, tumor cell cytotoxicity was observed for only 5 days with PCL meshes, whereas 10% PGC-C18 doped meshes showed low levels of killing out to 35 days.

### Example 21

### CPT-11 Loaded Electrospun Poly(Caprolactone) and Poly(Caprolactone) with Hydrophobic Dopant are Not Cytotoxic to Human Colorectal Cell Line

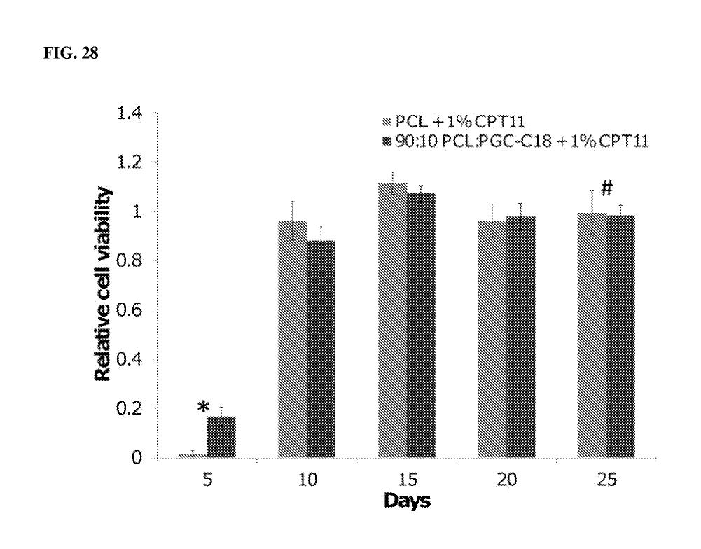

Given the low conversion of CPT-11 to SN-38 and the relatively rapid release, it was hypothesized that meshes loaded with 1 wt % CPT-11 would be less effective at killing tumor cells over a long period of time. Similar to the 0.01% SN-38 loaded meshes, in vitro tumor cytotoxicity for either CPT-11 loaded PCL or 10% PGC-C18 doped meshes was observed for only 5 days (FIG. 28). The viability trend for both PCL and PCL doped with 10% PGC-C18 is the same, and highlights that a much larger dose of CPT-11 is required to treat cancer cells when compared to SN-38. This finding demonstrates that delivery of the more potent active drug SN-38 from the superhydrophobic electrospun meshes is an attractive alternative to delivering the prodrug CPT-11 systemically or using a local drug delivery platform

### Example 22

### Uses of Electrospun Meshes

It is further contemplated herein that electrospun meshes can be produced as large sheets, specific sizes, and stapled with a surgical stapler.

### Example 23

### Electrospun Poly(Caprolactone) and Poly(Caprolactone) with Hydrophobic Polymer Dopant have Elastic Moduli Similar to Commercial Buttressing Materials

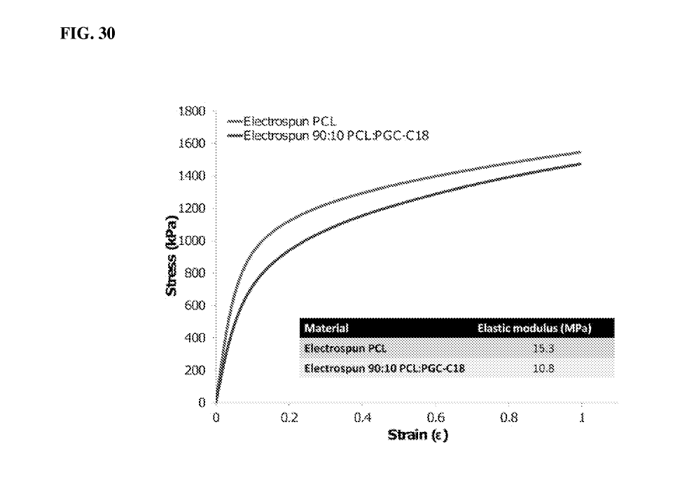

Strength was assessed in the axial direction by performing mechanical testing on strips of electrospun mesh (FIG. 30). PCL has an elastic modulus of 15.3 MPa, and with 10% PGC-C18 doping the modulus is 10.8 MPa. Without wishing to be bound by theory, this ˜30 decrease in elastic modulus may be a result of a change in porosity as a result of fiber size decrease, or addition of a lower molecular weight polymer with fewer chain entanglements. The ultimate tensile strengths (UTS) of both mesh types is ≈1.5 MPa (p-value=0.03) and compares well to other products proposed or evaluated for reinforcement after gastrointestinal surgery such as SEAMGUARD (UTS=4 MPa) with the goal of preventing such anastomotic complications as leakage and dehiscence.

### Example 24

### SN-38 Encapsulated within Electrospun Poly(Caprolactone) and Poly(Caprolactone) with a Hydrophobic Dopant is Protected from Hydrolysis

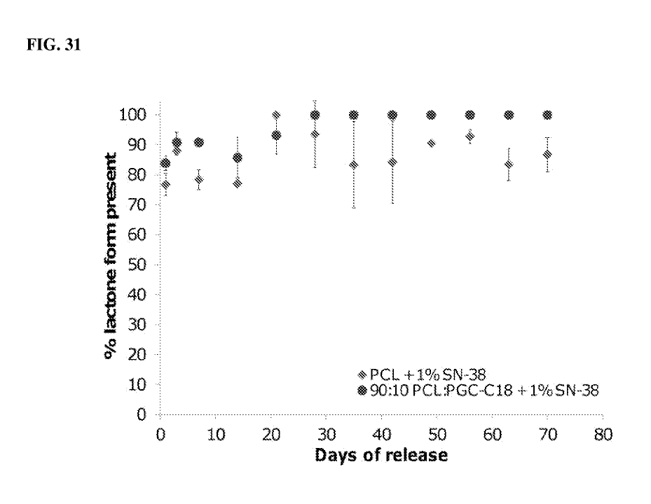

Another potential benefit of superhydrophobic electrospun meshes is a means to protect the active form of SN-38 and CPT-11 from hydrolysis, thereby keeping the lactone ring intact and the drug active. An additional release study was performed to determine if the active form of SN-38 was preserved within electrospun meshes. The lactone form is protected until released into PBS in both PCL and PCL doped with PGC-C18, with greater than 75% of SN-38 sampled from the release media being present in the lactone form (FIG. 31). This is compared to 73% of SN-38 remaining in the active lactone form in the control experiments, where a bolus of SN-38 was added to pH 6.4 PBS for an hour.

### Example 25

### Prevention of Tumor Recurrence using Camptothecin-Loaded Meshes in a Lung Cancer Recurrence Model

Female C57BL/6 mice at six to eight weeks of age were obtained from Jackson Laboratories (Bar Harbor, Me.). A primary tumor was induced by subcutaneous injection of 7.5×10⁵ Lewis Lung Carcinoma (LLC) cells (in 0.2 mL PBS) on the dorsum of Female C57BL/6 via a 27-gauge needle attached to a 1 mL syringe. This tumor dose effectively results in rapidly progressive tumor within 2 weeks. Tumor volume was estimated by the formula (length×width×height×Pi)/6, and the primary tumor was surgically removed when the tumor reached 300 mm². This size was chosen as the majority of animals will develop locally recurrent tumor despite aggressive surgical resection if no additional therapeutic intervention is performed to prevent recurrent disease.

Unloaded or camptothecin-loaded meshes (1.0×0.8 cm; 10% w/w), similar to those described in Example 6, were implanted with the polymer abutting the area of surgical resection. The four corners of the mesh were sutured to the superficial fascia in order to secure the position of the strip and the skin incision is closed with 5-0 polypropylene sutures. Tumor controls were utilized where no additional therapy was given following surgical resection in order to establish the incidence of recurrence in these experiments. The resulting data indicate that camptothecin-loaded polymer blend meshes incorporated at the surgical margin, afford enhanced local drug delivery aimed at preventing the growth of occult disease present following parenchyma-sparing surgery, and offer the means to decrease local recurrence rates in patients with stage I-IIIa lung cancer in the future.

### Example 26

### High-Intensity Focused Ultrasound can be Used to Remove Air Entrapped from Poly(Caprolactone) and Poly(Caprolactone) Doped with a Hydrophobic Polymer Dopant

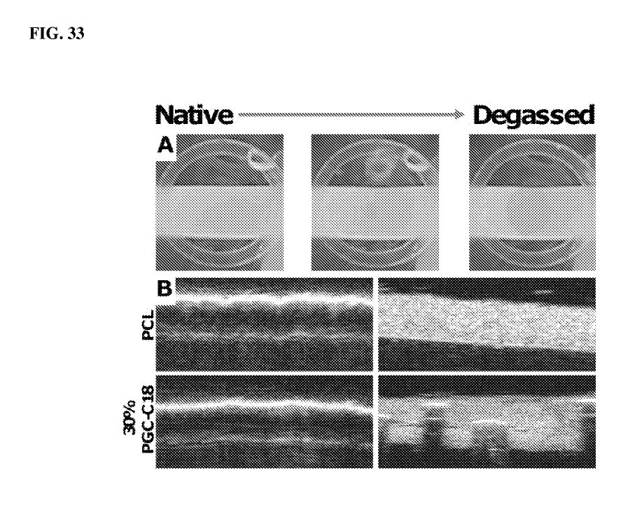

Meshes were fabricated similar to Example 1. The removal of air was studied using a three-prong approach after HIFU treatment: direct optical visualization, quantification of total wetted area, and B-mode imaging. Wetting of superhydrophobic meshes was directly visualized, where before treatment meshes were opaque with an entrapped air layer, and light is easily scattered/reflected (FIG. 33A). Application of a sufficient acoustic pressure using HIFU resulted in removal of the entrapped air, which was visualized directly by removal of the air bubbles, as well as increased transparency at the site of treatment.

Differences in the wetting behavior are also seen in cross section using B-mode imaging (VisualSonics, Inc, 55 MHz scanhead). Without wetting, images of submerged meshes had a bright interface, where the entrapped air highlighted the surface rather than the underlying porous 3D structure. However, the bulk mesh was easily visualized with removal of the entrapped air layer using HIFU, where the material appeared bright, as the electrospun fibers within the mesh created a large number of scattering sites (FIG. 33B). The weakly metastable entrapped air layer with PCL was fully removed and the entire mesh was visualized. With 30% PGC-C18 addition to PCL, the majority of air was removed, but some air bubbles/pockets remained and prevented full transmission of ultrasound for visualization of the hydrophobic mesh.

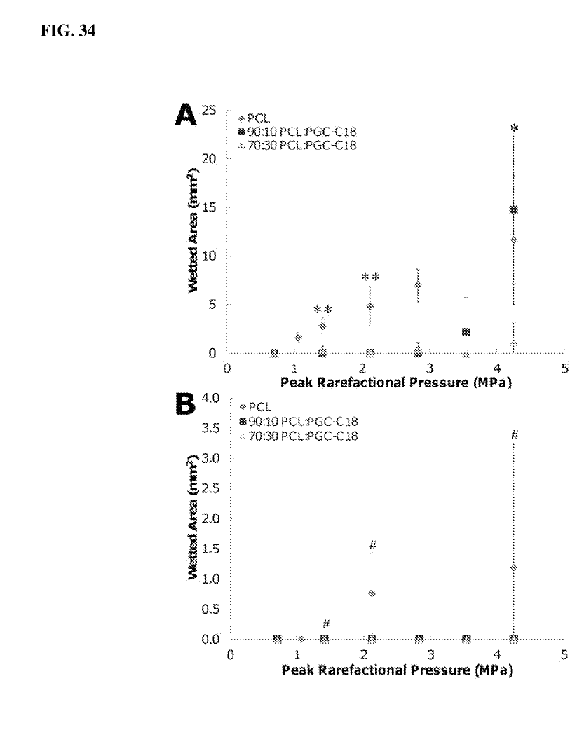

Video recordings taken during HIFU treatment were subsequently analyzed to determine the total wetted area of 3D superhydrophobic meshes. A reference frame of meshes before treatment was used for background subtraction, and the resultant change in image intensity after HIFU treatment was used to calculate the total wetted area. HIFU treatment was performed for 10 seconds in continuous wave (CW) mode or pulsed mode (center frequency of 1.1 MHz, pulse duration of 10 cycles, pulse repetition frequency of 50 Hz) using PCL, PCL with 10% PGC-C18, and PCL with 30% PGC-C18 meshes with peak rarefaction pressures ranging from 0.71-4.25 MPa. Undoped PCL meshes were easily wetted by HIFU in CW mode with peak rarefaction pressures of 1.06 MPa and higher, with a linear increase in the wetted area (FIG. 34A). With application of 4.25 MPa of pressure, a maximum area of 11.6 mm² was wetted. Superhydrophobic meshes containing 10% or 30% PGC-C18 required a 3-4 fold increase in applied pressure to induce wetting and remove the entrapped air. With 10% PGC-C18 addition, the minimum applied peak rarefaction pressure to achieve wetting was 3.54 MPa, with significant wetting observed at 4.25 MPa (14.8 mm²). With 30% PGC-C18 addition, only a modest amount of wetting was present at the highest pressures used (1.17 mm² at 4.25 MPa). Significantly different results were obtained when using HIFU in pulsed mode. With the addition of any PGC-C18 to PCL meshes, no wetting was observed in pulsed mode. PCL meshes which did not contain PGC-C18 still wetted at all intensities, but show ≈10-fold less wetting compared to CW mode. The decrease in wetting when moving from CW mode to pulsed mode indicates that removal of entrapped air is also a function of the total ultrasound exposure time. While the peak rarefaction pressures are the same for both treatments, the total on-time of ultrasound transmission was 22,000 times greater in CW mode than in pulsed mode.

### Example 27

### Air Removed with High Intensity Focused Ultrasound from Non-Woven Poly(Caprolactone) Meshes Doped with a Hydrophobic Polymer Dopant Triggers Drug Release

HIFU treatment was used as a trigger to initiate drug release from 3D superhydrophobic meshes. SN-38 (7-ethyl-10-hydroxycamptothecin) was selected as a model drug for use in these studies due to its potency in treating many cancer types, the relative ease in detecting low quantities (<1 ng/mL), and is the active metabolite of irinotecan. SN-38 (0.1 wt % and 1 wt %) was encapsulated into PCL with 30% PGC-C18 meshes, which have a stable air layer over several months (>10 weeks) when placed in an aqueous solution.

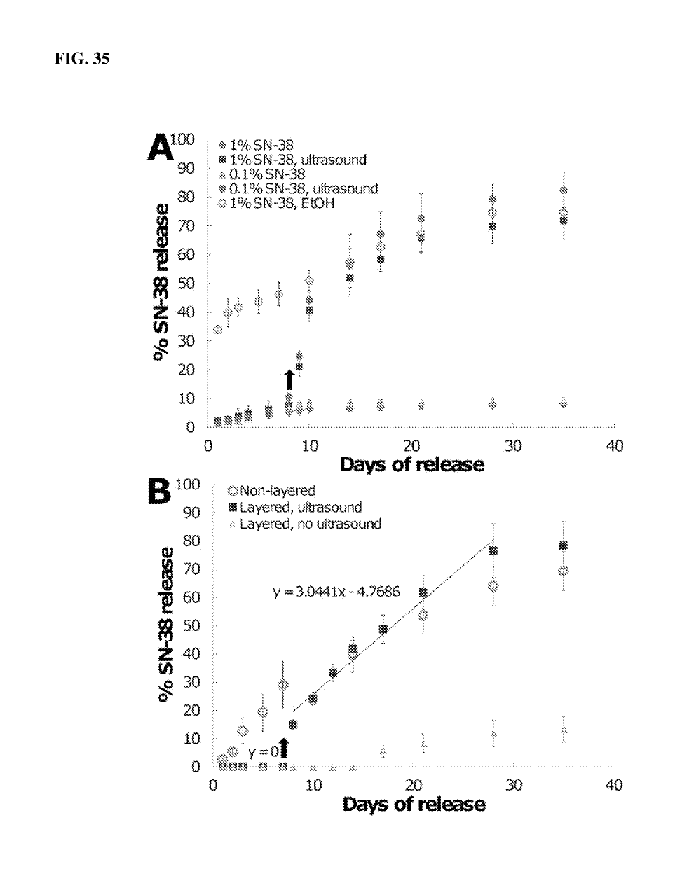

In the first study, less than 10% of SN-38 was released over 35 days without ultrasound treatment for meshes containing 0.1% or 1% SN-38 (FIG. 35A). However, with application of a sufficient HIFU treatment (4.25 MPa) at day 7, drug release was triggered/initiated with water infiltration into superhydrophobic meshes. More than 50% of total encapsulated SN-38 released 14 days after ultrasound treatment, followed by a slower, steady release of the remaining drug which concludes 28 days after treatment. Similar drug release profiles were observed for both concentrations of SN-38 used.

To further confirm that displacement of air leads to subsequent drug release, an ethanol dip study was done. Dipping in ethanol led to immediate removal of the air layer, as the surface tension of ethanol is significantly lower than water (22 vs. 72 mN/m). After a 5 second ethanol dip, meshes released SN-38 with an initial burst (>30% SN-38 in 2 days), with remaining drug released linearly over 4 weeks.

Next, the drug release study was repeated in the presence of serum, as proteins including albumin are expected to modify the release properties. Surfactants are well known to reduce the surface energy of a superhydrophobic surface through binding events of their hydrophobic domains, as well as reduction of the surface tension of applied water, both of which can lead to greater ease in removing entrapped air. When performing release in 10% serum, the entrapped air layer in meshes containing 30% PGC-C18 was no longer fully stable (FIG. 35B), with prolonged linear release over 35 days. However, the entrapped air layer still slowed water penetration compared to the fully wetted ethanol case (40% vs. 58% released at 14 days).

One strategy to mitigate immediate release in the presence of biological surfactants is to use a layer-by-layer construct, where two non-drug loaded layers sandwich the drug containing layer and act as a superhydrophobic barrier to effectively prevent release. A layer-by-layer construct was fabricated with a 100 μm drug-loaded interior, sandwiched between two non-drug loaded 120 μm meshes. All of the layers are created from PCL with 30% PGC-C18. For the first 14 days, no drug release was observed for untreated meshes (0% SN-38 release/day), with minimal drug release after (13% at 35 days) in the presence of serum. With an ultrasound treatment at 7 days, drug release was initiated (33% SN-38 in 5 days), with remaining drug released by 28 days (3% SN-38 release/day post ultrasound treatment). Differences in SN-38 release rates from layered meshes before and after ultrasound treatment were statistically significant using an analysis of covariance (ANCOVA) (p=0.0012).

### Example 28

### SN-38-Loaded Poly(Caprolactone) Meshes with a Hydrophobic Polymer Dopant are Cytotoxic Only when Triggered with Ultrasound

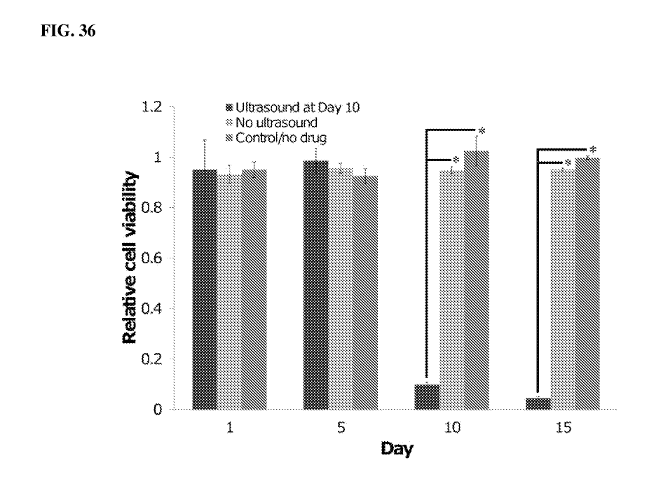

The layer-by-layer superhydrophobic meshes containing 1% of SN-38 in an in vitro cell assay using a human breast cancer cell line (MCF-7) in serum containing media. Cells incubated with SN-38-loaded meshes, which did not receive an ultrasound treatment, and empty non-drug loaded meshes, were viable for the 15 day study (FIG. 36). Cells incubated with SN-38 containing meshes prior to ultrasound treatment were fully viable, and with ultrasound treatment at day 10 afforded cell cytotoxicity at subsequent time points (p<0.0001).

### Example 29

### Formation of Poly(Caprolactone) Porous Coatings with and without a Hydrophobic Doping Agent

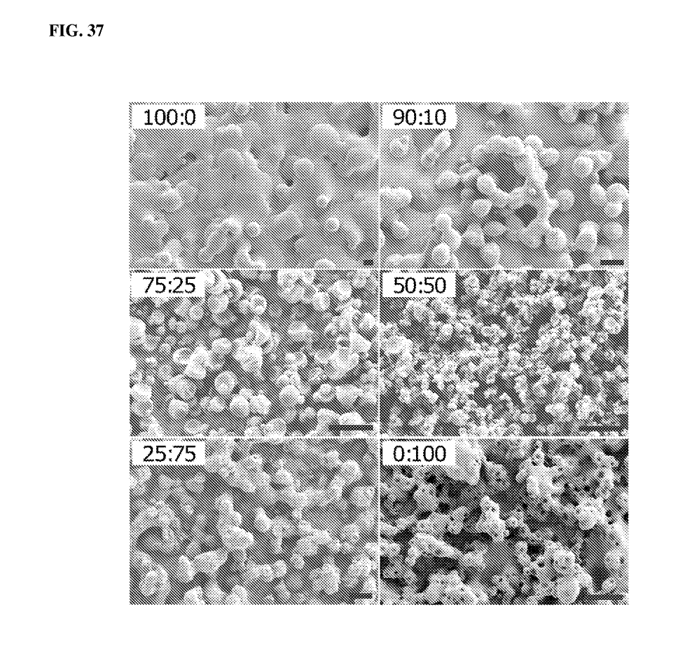

Connected electrosprayed particle coatings were fabricated from PCL and PGC-C18. Specifically, PCL (45 kD, 10 wt %, CHCl₃) was doped with varying amounts of PGC-C18 in order to modulate hydrophobicity and achieve the desired superhydrophobic state. The blended polymer solutions were electrosprayed (20 kV, 20 cm working distance (WD), 5 mL/hr, 18 G needle) on aluminum foil. Changing the PGC-C18 concentration from 0 to 100% produced varied particle sizes, particle textures, and 3D connectivity (FIG. 37). Higher concentrations of PCL resulted in deposits of large, wet particles due to a high solution viscosity, where a larger viscosity promotes low fragmentation of particles during the electrospraying process due to chain entanglement within the formed/drying droplets. Relatively flat surfaces for PCL and PCL with 5% PGC-C18 were observed, where deposition of these wet particles allowed almost total integration into one another. Increasing the PGC-C18 content reduced solution viscosity, as the molecular weight of PGC-C18 is lower than that of PCL (20 kD vs. 45 kD), and afforded smaller, less connected particles. Particle sizes ranged from 46±12 μm for PCL without PGC-C18 doping, to as small as 3.3±1.1 μm for 50% PGC-C18 doping. Electrosprayed coatings with >50% PGC-C18 showed a slight increase in particle size even with a continued drop in viscosity, where the waxy, low melting point PGC-C18 allows additional plasticity of the droplets to form larger more interconnected features once deposited. Electrospraying the PCL-PGC-C18 system produced a highly porous structure affording large apparent contact angles. Extremely hydrophobic coatings are formed (ACA=172°) with contact angle hysteresis as low as 5° (FIG. 31). Dual-scale roughness, with ripples and pores on individual particles, also contributes to these large contact angles. At these electrospraying conditions only modest additions of PGC-C18 are required for superhydrophobicity, and the addition of 25% PGC-C18 to PCL affords the maximum apparent contact angle for the system.

### Example 30

### Connectivity of Poly(Caprolactone) Porous Coatings with and without a Hydrophobic Doping Agent are Tuned with Electrospraying Parameters and Affects Mechanical Properties

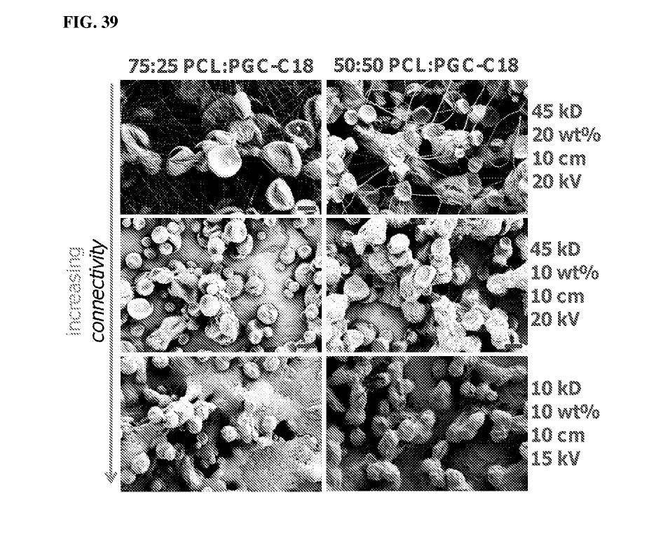

Electrospinning solution and processing parameters for 25% PGC-C18 and 50% PGC-C18 coatings were optimized to produce more robust, connective 3D superhydrophobic coatings by increasing the solvent present (i.e. wetness) when individual sprayed particles were deposited. These concentrations of PGC-C18 were selected to produce electrosprayed surfaces that were sufficiently hydrophobic/superhydrophobic. First, the electrospraying solution concentration was doubled to 20 wt % in addition to decreasing the WD to 10 cm from the initial parameters (FIG. 39). These changes resulted in large individual particles that were not directly connected, but were instead threaded together by thin fibers, forming a beads-on-a-string morphology. Second, reduction of the electrospinning solution concentration back to 10 wt % resulted in loss of the thin fibers, and microspheres were more heavily textured and minimally connected in three dimensions. Third, the PCL molecular weight was decreased from 45 kD to 10 kD, and the applied voltage decreased, resulting in wet particles being deposited and connected in 3D. All electrosprayed surfaces have advancing ACA>165°, with CA hysteresis<7° (FIG. 12).

Electrosprayed 75:25 PCL:PGC-C18 and 50:50 PCL:PGC-C18 coatings were then tested for mechanical robustness using ultrasonication and scotch tape delamination treatments. Electrosprayed surfaces were submerged in water, where an ultrasonication treatment was performed for 30 seconds, after which control and ultrasonicated samples were probed using contact angle measurements. Surfaces identified as not connected in 3D by SEM were easily sheared off from their aluminum substrates (see 75:25 and 50:50 PCL:PGC-C18 coatings in FIG. 10), where individual particles were freed from the surface and observed floating in solution during treatment. No apparent contact angle was formed for these materials post-ultrasound treatment, and the superhydrophobic characteristic of the surfaces was removed. Using less aggressive treatments (scotch tape or rubbing the surface) also resulted in removal of the loosely adhered particles from the coated surface. With an identical ultrasonication or scotch tape treatment, 3D superhydrophobic coatings which formed connected structures (see 75:25 and 50:50 PCL:PGC-C18 coatings in FIG. 20) retained their integrity and connectedness. The apparent contact angle remained unchanged before and after treatment.

More extreme ultrasonication conditions did not damage the coatings (20 minutes, 3× power), and only with aggressive sheer conditions with forceful scraping were such coatings damaged. Based on the above studies, the 50:50 PCL:PGC-C18 coatings (45 kD PCL, 10 wt %, 10 cm WD, 20 kV) were selected for further studies as a representative coating with favorable connectivity and superhydrophobicity.

### Example 31

### The Thickness of Poly(Caprolactone) Porous Coatings with and without a Hydrophobic Doping Agent Can be Modified

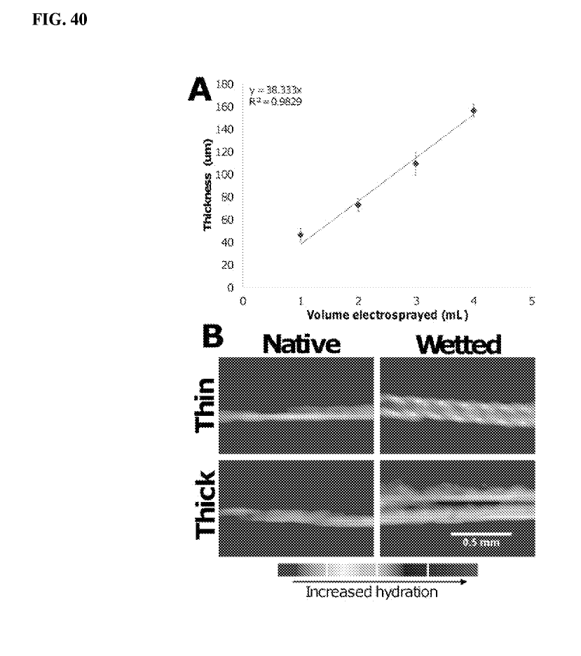

Different thickness 3D superhydrophobic coatings were then fabricated to investigate the three-dimensional nature of these electrosprayed coatings. By varying the time of deposition (and selected flow rate) the total thickness of the 3D superhydrophobic coating was controlled (FIG. 40A). The thinnest coating produced was 47 μm, and the thickest 156 μm. The expected linear trend between deposition time and thickness is shown in FIG. 21.

### Example 32

### Computed Tomography Shows that Poly(Caprolactone) Coatings with and without a Hydrophobic Doping Agent are Porous Throughout the Material

The superhydrophobic/3D nature of these surfaces was confirmed by imaging sample surface coatings (73 μm and 156 μm) using x-ray commuted tomography to create a 3D representation. First, electrosprayed surfaces were dipped in a saline-ioxaglate solution for 2 hours to show no contrast agent infiltration and to demonstrate superhydrophobicity over the entirety of the material surface (FIG. 40B), where only the aluminum foil-and-air interface on the underside of the coatings was observed when imaged. The 3D superhydrophobicity was then confirmed by an ethanol dip treatment to forcefully wet the electrosprayed coating, followed by immersion into the saline-Hexabrix solution to image the coating. After treatment, infusion of water into the 3D coatings was observed, demonstrating the existence of porosity within the 3D structure to support superhydrophobicity within the bulk material. Additionally, ethanol-treated coatings sank after removal of the entrapped air, where untreated coatings needed to be forcefully immersed during incubation.

### Example 33

### Poly(Caprolactone) Porous Coatings with and without a Hydrophobic Doping Agent Can Coated on Varied Material Types

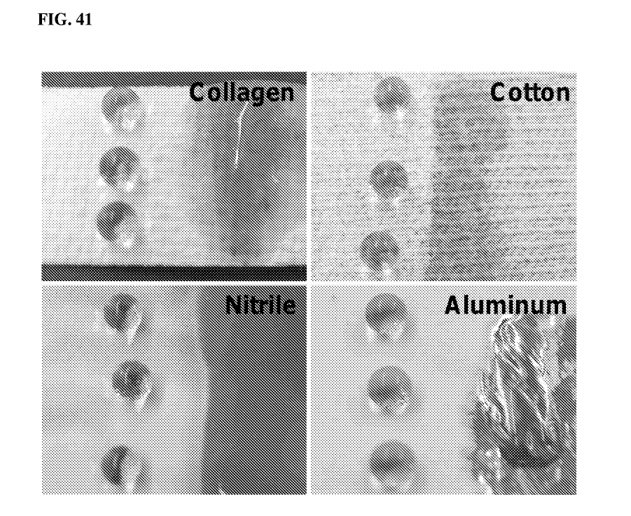

This 3D superhydrophobic electrosprayed coating technique is a substrate generic approach to coat structurally and compositionally different materials such as collagen, cotton fabric, nitrile rubber, and aluminum foil (FIG. 41). After electrospraying onto these surfaces, the resultant contact angle of all four surfaces is >167° (hysteresis<7°), whereas the uncoated portions of the material are easily and quickly wetted. Materials which are electrically insulating, such as glass, can be coated with the use of conductive copper tape near the material surface to ground the current used in the electrospraying process.

### Example 34

### Layer-by-Layer Poly(Caprolactone) Meshes and Poly(Caprolactone) Meshes with a Hydrophobic Polymer Dopant Delay SN-38 Release

The 3D nature of electrospun superhydrophobic materials can be further utilized by creating layered meshes so that each layer's polymer composition, thickness, and drug loading can vary the release kinetics. Clinically, chemotherapy is usually withheld for 14 days following a tumor resection surgery, so it would be beneficial to produce the same effect in vivo with a local drug delivery device. Accordingly, layered meshes were created with a drug-loaded polymer layer surrounded by two layers of polymer without drug, with the idea that the outer layers will delay wetting of the inner layer and therefore drug release.

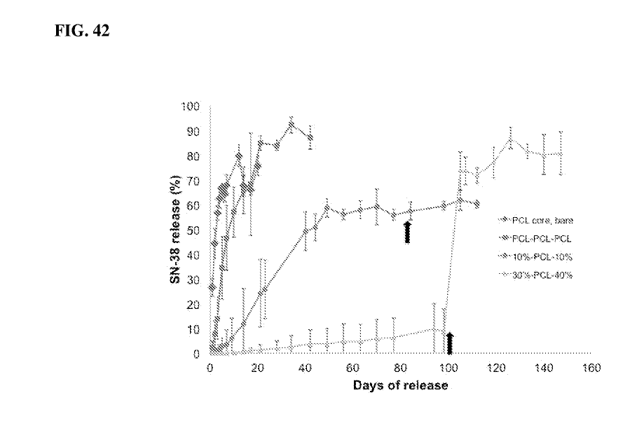

The layered meshes below were created with a 90-μm core of PCL with 1 wt % SN-38, with 150-μm unloaded layers above and below (FIG. 42). The polymer in the outer layers varied from pure PCL to a 70:30 PCL to PCG-18 blend. The meshes were incubated at 37° C., placed in either phosphate buffered saline (PBS) or 10% fetal bovine serum (FBS), and weighed down to force submergence. Media was changed to maintain sink conditions of less than 10% of drug solubility in the media. For comparison, release from a bare, un-layered core is also shown.

To confirm the mechanism of delayed release, some meshes were wetted by submerging in 95% ethanol and briefly (5 sec.) sonicating. The ethanol was then returned to the release media, which was mixed and immediately sampled. Black arrows in denote the dates of ethanol wetting.

### Example 35

### Layer-by-Layer Poly(Caprolactone) Meshes and Poly(Caprolactone) Meshes with a Hydrophobic Polymer Dopant Delay SN-38 Release in Serum Containing Media

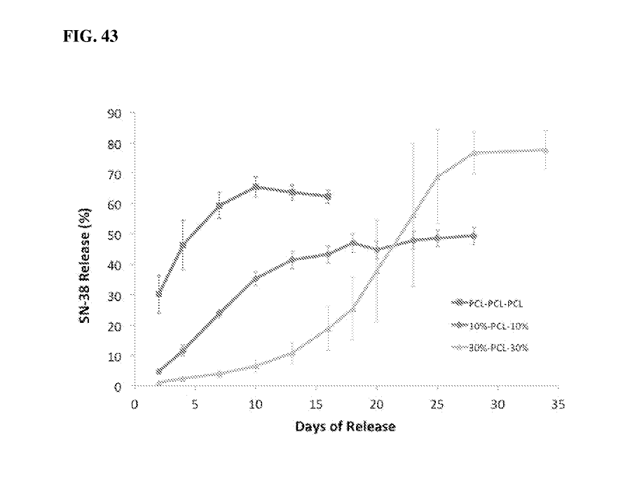

The release rate of SN-38 from meshes in an FBS solution was markedly faster than in PBS alone (FIG. 43). This is expected due to the additional proteins and surfactants lowering the air-media surface tension, and therefore destabilizing the entrapped air. However, the 30% shield condition exhibited a delay in wetting which compares well with the standard 14-day delay in chemotherapy following surgery.

### Example 36

### Poly(Glycerol-Co-ε-Caprolactone) is Functionalized with a NPE Photoactive Pendant Group

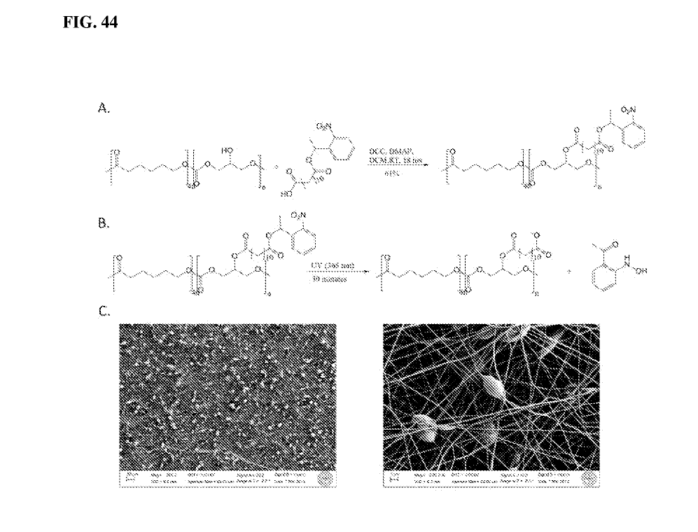

A poly(glycerol-co-ε-caprolactone) (1:4) (PGC) backbone was synthesized, and functionalized with a 12-(1-(2-nitrophenyl)ethoxy)-12-oxododecanoic acid (C12-NPE) side chain through an ester linkage to make a UV active polymer (FIG. 44). The PGC-C12-NPE polymer was mixed with poly(ε-caprolactone) (PCL) (70,000-90,000 MW, Sigma) at a 3:7 weight ratio as a 10% by weight 5:1 chloroform:methanol solution. The polymer blend was electrospun using parameters modified from a previous publication based on PCL. The mesh's surface was analyzed using a Zeiss SUPRA 55VP field emission scanning electron microscope (SEM) to identify micrometer (˜3-5 μm beads) and nanometer (fiber diameters ˜100-150 nm) scale textures on the materials surface.

### Example 37

### Poly(Caprolactone) Doped with Hydrophobic Photoactive Dopant Transitions from Hydrophobic to Hydrophilic with Light Exposure

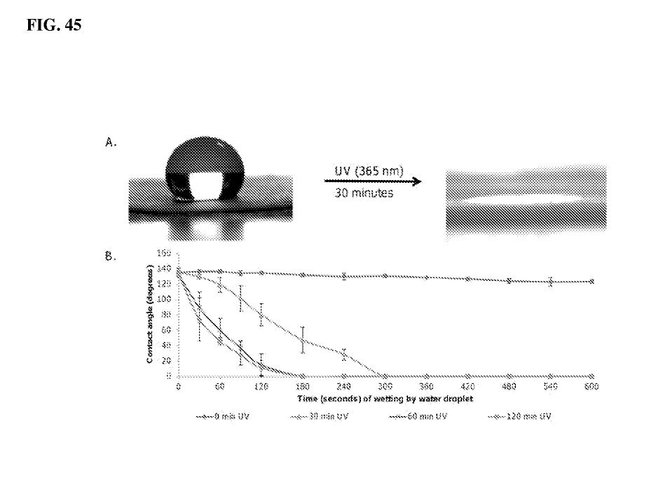

In order to determine the effects of UV light exposure, a 13 W spectroline long wave UV lamp (λ=365 nm, Spectroline, Westbury, N.Y.) was used to expose ˜80 μm thick meshes to UV light for 0, 15, 30, 60, 90, and 120 minutes. Four microliter water droplets were recorded at a 0.2 Hz frame rate on top of the meshes after each UV exposure time using a Kruss DSA100 contact angle goniometer. As expected, the photoactive electrospun PGC-C12-NPE meshes were found to exhibit a UV induced transition from hydrophobic ACA (˜135°) to hydrophilic ACA (˜0°) after various UV exposure times due to the NPE deprotection (FIG. 45). The meshes had a UV dose dependent wetting profile where smaller UV doses wetted more slowly over time compared to larger UV doses. With as little as 15 minutes of UV exposure, the ACA was shown to decrease dramatically over 10 minutes compared to the unexposed control. Doubling the UV exposure time resulted in more consistent ACAs and a fully wetted surface (ACA ˜0°) within 5 minutes. Maximum wetting rates were achieved with UV exposure times greater than 60 minutes where the films fully wetted within 2.5 minutes.

### Example 38

### Fraction of NPE Cleavage Increases with UV Exposure

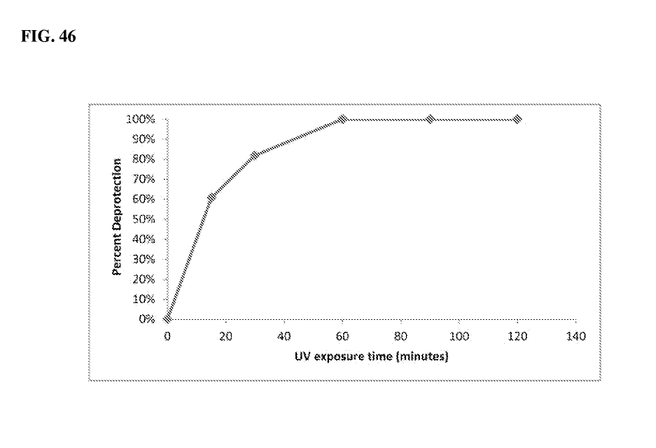

In order to confirm that after 60 minutes of UV exposure most of the NPE groups exposed to the UV light were cleaved, a Varian 400 MHz VNMRS NMR was used to track the NPE deprotection over time (FIG. 46). It was clear that after 60 minutes of UV exposure nearly all of the photocleavable groups in the ˜80 μm thick meshes were removed (˜100%).

### Example 39

### Deprotected Poly(Caprolactone) Doped Poly(Glycerol-Co-ε-Caprolactone-NPE) Meshes have Different Wetting Regimes

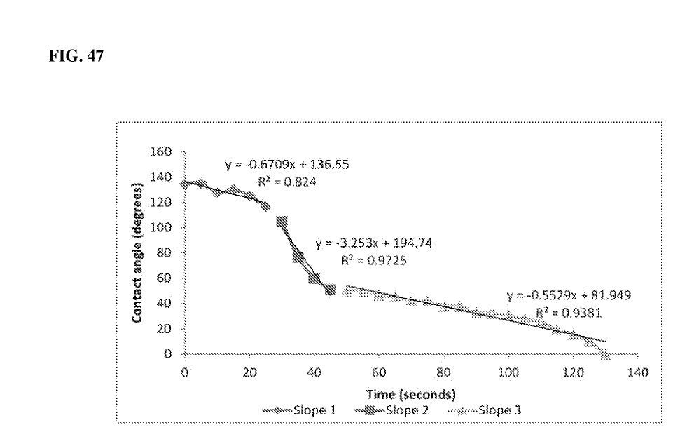

For a fully deprotected mesh, the initial wetting rate is dictated by the Cassie-Baxter to Wenzel transition wetting profile where air is slowly displaced by the water directly below the water droplet which progressively wets the surface roughness, associated with the nanometer to micrometer textures on the mesh's surface (FIG. 47). Once the droplets reach a critical ACA of ˜110°, the wetting rate dramatically increases by 4 fold as the contact angle drops to ˜50°. The ACA then continues to decrease at a similar rate as the initial wetting until it reaches ˜0°.

Interestingly, the contact angle of a cast film of the polymer has a contact angle of about 113° before UV irradiation and a contact angle of about 108° after UV irradiation which indicates the apparent contact angle of ˜135° before UV exposure is dramatically influenced by the micrometer and nanometer scale roughness of the meshes. Since the wetting rate rapidly increased when the apparent contact angle reached ˜110°, it is possible that this change is due to the apparent contact angle reaching the stable contact angle of this material when it is fabricated as a smooth surface. However, since the surface is not smooth, the roughness begins to exaggerate the hydrophilicity of the material causing the apparent contact angle to rapidly decrease to 50°. Ishino et al. describes this phenomenon of hydrophilic rough surfaces as a transition from the Cassie to Wenzel to Sunny-side-up state where the water begins to penetrate a rough surface beyond the boundaries of the water droplet above the surface such that it appears like a sunny-side-up egg. This theory is consistent with the three distinct wetting regimes that are observed as the apparent contact angle transitions from the Cassie to Wenzel state, the Wenzel to Sunny-side-up state (rapid), and the Sunny-side-up to fully wetted state as the water continues to penetrate into the rough porous material until there is no volume above the surface of the material.

### Example 40

### UV Exposure to Poly(Caprolactone) Doped Poly(Glycerol-Co-ε-Caprolactone-NPE) Meshes Causes Water Infiltration

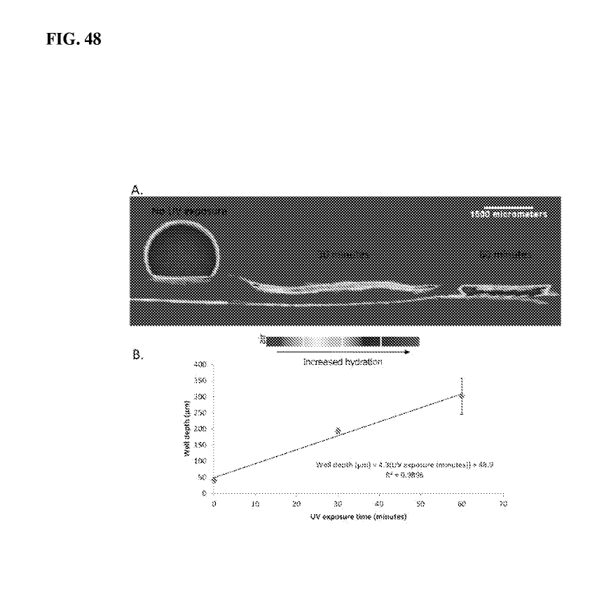

In order to determine the utility of this photo-labile system as a facile method for printing 3D hydrophilic regions surrounded by hydrophobic regions, a circular photo mask (1590 μm in diameter) was used to create 3D hydrophilic cavities of various depths within the hydrophobic bulk material by varying the UV exposure time (FIG. 48). These hydrophilic regions were analyzed by applying a solution of a water soluble CT contrast agent (Visipaque, GE Healthcare) to the surface of the meshes and using a μCT scanner to measure the water penetration into the meshes (see SI for details). If the film is not exposed to UV light the CT contrast agent is restricted to the surface of the hydrophobic mesh. Using the circular photomask, 194.2±8.2 μm and 301.1±55.7 μm deep cavities were fabricated by exposing the UV active meshes to UV light for 30 minutes and 60 minutes, respectively. A linear relationship between the UV exposure time and the depth of the cavities was determined; however, the diameter of the cavities was not as well defined. The photomask was 1590 μm in diameter while the average cavity diameters were 5803.9±138.1 μm and 2709.1±485.2 μm for the 30 minute and 60 minute exposure times respectively (n=3). This indicates that the hydrophobicity of the material is anisotropic were layers through the thickness of the material are hydrophobic but across the plane of any given layer, the material's hydrophobicity is dramatically reduced.

### Example 41

### Porous Hydrophobic Electrospun Meshes Selectively Absorb Oil

Due to the high porosities of the non-woven polymeric electrospun meshes and the relatively low surface tension associated with various polymer dopants mentioned above, these constructs can be used to separate oil out of an oil/water emulsion by preferentially wetting with oil over water and be capable of removing large volumes of oil from an emulsion. Variations in mesh geometries, fiber diameters, surface tensions, and porosities can be used to tune the constructs for a variety of oil/water separation applications depending on the intended outcome. Certain applications may require a high degree of water purification where removing oil and water is acceptable as long as the remaining water is pure. Other applications may require a pure oil sample in which case the constructs should exclusively separate oil out of the emulsions.

### Example 42

### Non-Woven Poly(Caprolactone) Meshes and Poly(Caprolactone) Meshes with a Hydrophobic Polymer Dopant do not Degrade in 3 Months

These electrospun meshes are flexible and deformable, making them ideal for fitting the contours of the pelvis, or for approximating the rectum or colonic flexures. Additionally, both PCL and PGC-C18 will not degrade significantly in 3 months, which will provide mechanical stability to the anastomosis during the 3-6 week healing phase. This was confirmed for these superhydrophobic electrospun meshes by demonstrating the absence of weight or structural changes after incubating meshes in PBS at 37° C. for three months.

1. A 3 dimensional composition comprising: a) a biodegradable polymer; b) one or more bioactive agents; and c) a hydrophobic doping agent,
wherein the composition comprises a plurality of surfaces that comprise a surface hydrophobicity that is substantially homogenous, and
wherein the composition comprises entrapped air.

2.-54. (canceled)
